(19) United States
(12) Patent Application Publication
(10) Pub. No.: US 2026/0078378 A1
(43) Pub. Date: Mar. 19, 2026

(54) Treatment Of Sepsis With PCSK9 And LDLR Modulators

(71) Applicant: Regeneron Pharmaceuticals, Inc., Tarrytown, NY (US)

(72) Inventors: Julie Horowitz, Tarrytown, NY (US); Luca Andrea Lotta, Tarrytown, NY (US); Manuel Allen Revez Ferreira, Tarrytown, NY (US); Amy Damask, Tarrytown, NY (US); Charles Paulding, Tarrytown, NY (US); Garen Manvelian, Tarrytown, NY (US); Michael Cantor, Tarrytown, NY (US); Aris Baras, Tarrytown, NY (US)

(21) Appl. No.: 19/256,467

(22) Filed: Jul. 1, 2025

Related U.S. Application Data
(63) Continuation of application No. 17/411,460, filed on Aug. 25, 2021, now Pat. No. 12,378,557.
(60) Provisional application No. 63/069,789, filed on Aug. 25, 2020.

(51) Int. Cl. C12N 15/113; A61P 31/00; C12N 9/22; C12N 15/11; C12Q 1/6883

(52) U.S. Cl. CPC C12N 15/1137; A61P 31/00; C12N 9/22; C12N 15/11; C12Q 1/6883; C12N 2310/20; C12Q 2600/156

(57) ABSTRACT

The present disclosure provides methods of treating subjects having sepsis, SIRS, septic shock, and/or MODS, methods of identifying subjects having an increased risk of developing sepsis, SIRS, septic shock, and/or MODS, and methods of detecting Proprotein Convertase Subtilisin/Kexin Type 9 (PCSK9) and/or Low Density Lipoprotein Receptor (LDLR) variant nucleic acid molecules and variant polypeptides.

### REFERENCE TO SEQUENCE LISTING

This application includes a Sequence Listing submitted electronically as an XML file named 381204405SEQ, created on Apr. 15, 2025, with a size of 319,967 bytes. The Sequence Listing is incorporated herein by reference.

### FIELD

The present disclosure relates generally to the treatment of subjects having sepsis, systemic inflammatory response syndrome (SIRS), septic shock, and/or multiple organ dysfunction syndrome (MODS) with a Proprotein Convertase Subtilisin/Kexin Type 9 (PCSK9) inhibitor and/or a Low Density Lipoprotein Receptor (LDLR) agonist, methods of identifying subjects having an increased risk of developing sepsis, SIRS, septic shock, and/or MODS, and methods of detecting PCSK9 and/or LDLR variant nucleic acid molecules and variant polypeptides.

### BACKGROUND

Sepsis is a systemic inflammatory response to infection that can lead to organ failure and death. The annual incidence of sepsis is greater than 48 million cases responsible for about 20% of deaths worldwide (Rudd et al., Lancet, 2020, 395, 200-2011). Bacterial cell wall components, including lipopolysaccharide (LPS) and lipoteichoic acid (LTA) for gram-negative bacteria and gram-positive bacteria, respectively, are key mediators of sepsis and septic shock. In healthy individuals, LPS/LTA are incorporated into lipoprotein particles, including LDL and HDL, and are cleared from the plasma via the LDL receptor (LDLR) in hepatocytes as a primary step in sequestering pathogen toxins from circulation. In general, sepsis is two or more systemic inflammatory response criteria plus a known or suspected infection. Severe sepsis is sepsis with acute organ dysfunction. Sepsis can progress to severe sepsis when, in addition to signs of sepsis, there are signs of organ dysfunction, such as difficulty breathing (problems with the lungs), low or no urine output (kidneys), abnormal liver tests (liver), and changes in mental status (brain). Nearly all patients with severe sepsis require treatment in an intensive care unit (ICU). Septic shock is the most severe level and is diagnosed when a subject's blood pressure drops to dangerous levels.

Systemic inflammatory response syndrome (SIRS) is an exaggerated defense response of the body to a noxious stressor (infection, trauma, surgery, acute inflammation, ischemia or reperfusion, or malignancy to name a few) to localize and then eliminate the endogenous or exogenous source of the insult. It involves the release of acute-phase reactants which are direct mediators of widespread autonomic, endocrine, hematological and immunological alteration in a subject. The dysregulated cytokine storm has the potential to cause massive inflammatory cascade leading to reversible or irreversible end-organ dysfunction and death.

During sepsis, systemic hypotension, disturbed perfusion of the microcirculation, and direct tissue-toxicity caused by inflammatory immune reaction can occur and contribute to organ failure. The failure of two or more vital organ systems is termed multi-organ dysfunction syndrome (MODS) and resembles a very critical condition associated with high morbidity and mortality. Importantly, no specific treatment strategy exists to efficiently prevent the development of MODS during sepsis.

Proprotein convertase subtilisin/kexin type 9 is an enzyme (encoded by PCSK9) that binds LDLR and prevents recycling of LDLR to the cell surface. It has been observed that the addition of exogenous PCSK9 protein reduced LPS/LTA uptake into cells in vitro (Boyd, Inn. Immunol., 2016; Grin, Sci. Rep., 2018; and Leung, Sci. Rep., 2019)

### SUMMARY

The present disclosure provides methods of treating a subject having sepsis or severe sepsis, the method comprising administering a PCSK9 inhibitor and/or an LDLR agonist to the subject in need thereof.

The present disclosure also provides methods of treating a subject having SIRS, the method comprising administering aPCSK9 inhibitor and/or an LDLR agonist to the subject in need thereof.

The present disclosure also provides methods of treating a subject having septic shock, the method comprising administering a PCSK9 inhibitor and/or an LDLR agonist to the subject in need thereof.

The present disclosure also provides methods of treating a subject having MODS, the method comprising administering a PCSK9 inhibitor and/or an LDLR agonist to the subject in need thereof.

The present disclosure also provides methods of treating a subject with a therapeutic agent that treats or inhibits sepsis, SIRS, septic shock, and/or MODS, wherein the subject is suffering from sepsis, SIRS, septic shock, and/or MODS, the method comprising the steps of: determining whether the subject has: i) a PCSK9 variant nucleic acid molecule encoding a PCSL9 predicted loss-of-function polypeptide; and/or ii) an LDLR variant nucleic acid molecule encoding an LDLR predicted loss-of-function polypeptide; by: obtaining or having obtained a biological sample from the subject; and performing or having performed a sequence analysis on the biological sample to determine if the subject has a genotype comprising: i) the PCSK9 variant nucleic acid molecule; and/or ii) the LDLR variant nucleic acid molecule; and when the subject is PCSK9 reference and LDLR reference, then also administering or continuing to administer to the subject the therapeutic agent that treats or inhibits sepsis, SIRS, septic shock, and/or MODS in a standard dosage amount, and administering to the subject a PCSK9 inhibitor and/or an LDLR agonist; when the subject is LDLR reference and heterozygous for the PCSK9 variant nucleic acid molecule, then also administering or continuing to administer to the subject the therapeutic agent that treats or inhibits sepsis, SIRS, septic shock, and/or MODS in an amount that is the same as or less than a standard dosage amount, and administering to the subject a PCSK9 inhibitor and/or an LDLR agonist; when the subject is PCSK9 reference and heterozygous for the LDLR variant nucleic acid molecule, then also administering or continuing to administer to the subject the therapeutic agent that treats or inhibits sepsis, SIRS, septic shock, and/or MODS in an amount that is the same as or less than a standard dosage amount, and administering to the subject a PCSK9 inhibitor and/or an LDLR agonist; when the subject is LDLR reference and homozygous for the PCSK9 variant nucleic acid molecule, then also administering or continuing to administer to the subject the therapeutic agent that treats or inhibits sepsis, SIRS, septic shock, and/or MODS in an amount that is the same as or less than a standard dosage amount, and administering to the subject an LDLR agonist; when the subject is PCSK9 reference and homozygous for the LDLR variant nucleic acid molecule, then also administering or continuing to administer to the subject the therapeutic agent that treats or inhibits sepsis, SIRS, septic shock, and/or MODS in an amount that is the same as or less than a standard dosage amount, and administering to the subject a PCSK9 inhibitor; when the subject is heterozygous for both the PCSK9 variant nucleic acid molecule and the LDLR variant nucleic acid molecule, then also administering or continuing to administer to the subject the therapeutic agent that treats or inhibits sepsis, SIRS, septic shock, and/or MODS in an amount that is the same as or less than a standard dosage amount, and administering to the subject a PCSK9 inhibitor and/or an LDLR agonist; and when the subject is homozygous for both the PCSK9 variant nucleic acid molecule and the LDLR variant nucleic acid molecule, the subject is also administered the therapeutic agent that treats or inhibits sepsis, SIRS, septic shock, and/or MODS in a dosage amount that is the same as or less than a standard dosage amount; wherein the presence of a genotype having both the PCSK9 variant nucleic acid molecule encoding a PCSL9 predicted loss-of-function polypeptide, and the LDLR variant nucleic acid molecule encoding an LDLR predicted loss-of-function polypeptide indicates the subject has a decreased risk of developing sepsis, SIRS, septic shock, and/or MODS.

The present disclosure also provides methods of identifying a subject having a risk of developing sepsis, SIRS, septic shock, and/or MODS, the method comprising: determining or having determined the presence or absence of a PCSK9 variant nucleic acid molecule encoding a PCSL9 predicted loss-of-function polypeptide, and/or an LDLR variant nucleic acid molecule encoding an LDLR predicted loss-of-function polypeptide in a biological sample obtained from the subject; wherein when the subject is PCSK9 reference and LDLR reference, the subject has an increased risk of developing sepsis, SIRS, septic shock, and/or MODS; and when the subject is heterozygous or homozygous for an LDLR variant nucleic acid molecule, and/or heterozygous or homozygous for a PCSK9 variant nucleic acid molecule, the subject has a decreased risk of developing sepsis, SIRS, septic shock, and/or MODS.

The present disclosure also provides methods of detecting a PCSK9 variant nucleic acid molecule in a subject comprising assaying a sample obtained from the subject to determine whether a nucleic acid molecule in the sample is: a genomic nucleic acid molecule comprising a nucleotide sequence comprising a thymine at a position corresponding to position 427 according to SEQ ID NO:2, or the complement thereof; an mRNA molecule having a nucleotide sequence comprising a uracil at a position corresponding to: position 428 according to SEQ ID NO:17, or the complement thereof; position 217 according to SEQ ID NO:18, or the complement thereof; or position 137 according to SEQ ID NO:19, or the complement thereof; or a cDNA molecule produced from an mRNA molecule, wherein the cDNA molecule has a nucleotide sequence comprising a thymine at a position corresponding to: position 428 according to SEQ ID NO:32, or the complement thereof; position 217 according to SEQ ID NO:33, or the complement thereof; or position 137 according to SEQ ID NO:34, or the complement thereof.

The present disclosure also provides methods of detecting an LDLR variant nucleic acid molecule in a subject comprising assaying a sample obtained from the subject to determine whether a nucleic acid molecule in the sample is a genomic nucleic acid molecule comprising a nucleotide sequence comprising a thymine at a position corresponding to position 2,269 according to SEQ ID NO:4, or the complement thereof.

The present disclosure also provides methods of detecting the presence of a PCSK9 Arg46Leu polypeptide, comprising performing an assay on a sample obtained from a subject to determine whether a PCSK9 protein in the sample comprises a leucine at a position corresponding to position 46 according to SEQ ID NO:42.

The present disclosure also provides therapeutic agents that treat or inhibit sepsis, systemic inflammatory response syndrome (SIRS), septic shock, and/or multiple organ dysfunction syndrome (MODS), for use in the treatment of sepsis, SIRS, septic shock, and/or MODS in a subject having: a genomic nucleic acid molecule having a nucleotide sequence encoding a Proprotein Convertase Subtilisin/Kexin Type 9 (PCSK9) polypeptide, wherein the nucleotide sequence comprises a thymine at a position corresponding to position 427 according to SEQ ID NO:2, or the complement thereof; and/or a genomic nucleic acid molecule having a nucleotide sequence encoding a Low Density Lipoprotein Receptor (LDLR) polypeptide, wherein the nucleotide sequence comprises a thymine at a position corresponding to position 2,269 according to SEQ ID NO:4, or the complement thereof; an mRNA molecule having a nucleotide sequence encoding a PCSK9 polypeptide, wherein the nucleotide sequence comprises a uracil at a position corresponding to: position 428 according to SEQ ID NO:17, or the complement thereof; position 217 according to SEQ ID NO: 18, or the complement thereof; or position 137 according to SEQ ID NO:19, or the complement thereof; or a cDNA molecule having a nucleotide sequence encoding a PCSK9 polypeptide, wherein the nucleotide sequence comprises a thymine at a position corresponding to: position 428 according to SEQ ID NO:32, or the complement thereof; position 217 according to SEQ ID NO:33, or the complement thereof; or position 137 according to SEQ ID NO:34, or the complement thereof.

The present disclosure also provides Proprotein Convertase Subtilisin/Kexin Type 9 (PCSK9) inhibitors and/or Low Density Lipoprotein Receptor (LDLR) agonists for use in the treatment of sepsis, systemic inflammatory response syndrome (SIRS), septic shock, and/or multiple organ dysfunction syndrome (MODS), in a subject that: a) is reference for a PCSK9 and/or an LDLR genomic nucleic acid molecule, a PCSK9 and/or an LDLR mRNA molecule, or a PCSK9 and/or an LDLR cDNA molecule; or b) is heterozygous for: i) a genomic nucleic acid molecule having a nucleotide sequence encoding a PCSK9 polypeptide, wherein the nucleotide sequence comprises a thymine at a position corresponding to position 427 according to SEQ ID NO:2; and/or ii) a genomic nucleic acid molecule having a nucleotide sequence encoding an LDLR polypeptide, wherein the nucleotide sequence comprises a thymine at a position corresponding to position 2,269 according to SEQ ID NO:4; an mRNA molecule having a nucleotide sequence encoding a PCSK9 polypeptide, wherein the nucleotide sequence comprises a uracil at a position corresponding to: position 428 according to SEQ ID NO:17, or the complement thereof; position 217 according to SEQ ID NO: 18, or the complement thereof; or position 137 according to SEQ ID NO:19, or the complement thereof; or a cDNA molecule having a nucleotide sequence encoding a PCSK9 polypeptide, wherein the nucleotide sequence comprises a thymine at a position corresponding to: position 428 according to SEQ ID NO:32, or the complement thereof; position 217 according to SEQ ID NO:33, or the complement thereof; or position 137 according to SEQ ID NO:34, or the complement thereof.

### BRIEF DESCRIPTION OF THE DRAWINGS

The accompanying figures, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows summary data of a meta-analysis of PCSK9 p.R46L and 28-day mortality across all tested cohorts with severe sepsis.

FIG. 2 shows genetically-predicted reduction in LDL levels and sepsis mortality rate based on genotype groups (calculated as: N_Alt_alleles_rs11591147×LDL_weight_rs11591147+N_Alt_alleles_rs6511720×LDL_weight_rs6511720; SNP weights were jointly estimated using linear regression (covariates: age and sex) in the UKB500K, with LDL levels measured on the raw scale (mg/dL); weights were −11.7 for the PCSK9 variant, and −5.9 for the LDLR variant).

FIG. 3 shows genetically-predicted reduction in LDL levels and sepsis mortality rate based on 10 mg/dl groups.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

Rare variants in the PCSK9 and LDLR genes associated with a decreased risk of developing sepsis, SIRS, septic shock, and/or MODS in subjects has been identified in accordance with the present disclosure. For example, a genetic alteration that changes the guanine nucleotide of position 427 in the human PCSK9 reference (see, SEQ ID NO:1) to a thymine and/or a genetic alteration that changes the guanine nucleotide of position 2,269 in the human LDLR reference (see, SEQ ID NO:3) to a thymine has been observed to indicate that the human having such alterations may have a decreased risk of developing sepsis, severe sepsis, SIRS, septic shock, and/or MODS. It is believed that no such variants of the PCSK9 and LDLR genes have any known association with sepsis, severe sepsis, SIRS, septic shock, and/or MODS. Altogether, the genetic analyses described herein surprisingly indicate that the PCSK9 gene and LDLR gene and, in particular, a variant in the PCSK9 gene and LDLR gene, associates with a decreased risk of developing sepsis, severe sepsis, SIRS, septic shock, and/or MODS. Therefore, subjects that are PCSK9 and/or LDLR reference that have an increased risk of developing sepsis, SIRS, septic shock, and/or MODS, may be treated such that sepsis, severe sepsis, SIRS, septic shock, and/or MODS is prevented, the symptoms thereof are reduced, and/or development of symptoms is repressed. Accordingly, the present disclosure provides methods of leveraging the identification of such variants in subjects to identify or stratify risk in such subjects of developing sepsis, severe sepsis, SIRS, septic shock, and/or MODS, or to diagnose subjects as having an increased risk of developing sepsis, severe sepsis, SIRS, septic shock, and/or MODS, such that subjects at risk or subjects with active disease may be treated accordingly.

For purposes of the present disclosure, any particular subject can be categorized as having any one of the following PCSK9 and LDLR genotypes: i) PCSK9 reference and LDLR reference; ii) heterozygous or homozygous for a PCSK9 variant nucleic acid molecule encoding a PCSK9 predicted loss-of-function polypeptide and LDLR reference; iii) heterozygous or homozygous for an LDLR variant nucleic acid molecule encoding an LDLR predicted loss-of-function polypeptide and a PCSK9 reference; or iii) heterozygous or homozygous for both a PCSK9 variant nucleic acid molecule encoding a PCSK9 predicted loss-of-function polypeptide and an LDLR variant nucleic acid molecule encoding an LDLR predicted loss-of-function polypeptide. A subject is a PCSK9 and/or an LDLR reference when the subject does not have a copy of a PCSK9 and/or an LDLR variant nucleic acid molecule. A subject is heterozygous for a PCSK9 and/or an LDLR variant nucleic acid molecule when the subject has a single copy of a PCSK9 and/or an LDLR variant nucleic acid molecule. As used herein, a PCSK9 and/or an LDLR variant nucleic acid molecule is any PCSK9 and/or any LDLR nucleic acid molecule (such as, a genomic nucleic acid molecule, an mRNA molecule, or a cDNA molecule) having a genetic variation described herein, encoding a polypeptide having a partial loss-of-function, a complete loss-of-function, a predicted partial loss-of-function, or a predicted complete loss-of-function. A subject who has a PCSK9 polypeptide having a partial loss-of-function (or predicted partial loss-of-function) is hypomorphic for a PCSK9. The PCSK9 variant nucleic acid molecule can be any nucleic acid molecule encoding a PCSK9 Arg46Leu polypeptide. A subject is homozygous for a PCSK9 and/or an LDLR variant nucleic acid molecule when the subject has two copies of a PCSK9 and/or an LDLR variant nucleic acid molecule.

For subjects that are genotyped or determined to be PCSK9 reference and LDLR reference, such subjects have an increased risk of developing sepsis, severe sepsis, SIRS, septic shock, and/or MODS. For subjects that are genotyped or determined to be heterozygous for a PCSK9 variant nucleic acid molecule and/or heterozygous for an LDLR variant nucleic acid molecule, such subjects can be treated with a PCSK9 inhibitor and/or an agent that reduces LDL, such as an LDLR agonist.

In any of the embodiments described herein, the PCSK9 variant nucleic acid molecule and/or the LDLR variant nucleic acid molecule can be any PCSK9 nucleic acid molecule and/or any LDLR nucleic acid molecule (such as, for example, genomic nucleic acid molecule, mRNA molecule, or cDNA molecule) encoding a polypeptide having a partial loss-of-function, a complete loss-of-function, a predicted partial loss-of-function, or a predicted complete loss-of-function. For example, the PCSK9 variant nucleic acid molecule can be any nucleic acid molecule encoding a PCSK9 Arg46Leu polypeptide.

In any of the embodiments described herein, the PCSK9 predicted loss-of-function polypeptide can be any PCSK9 polypeptide having a partial loss-of-function, a complete loss-of-function, a predicted partial loss-of-function, or a predicted complete loss-of-function. In any of the embodiments described herein, the PCSK9 predicted loss-of-function polypeptide can be any of the PCSK9 polypeptides described herein including, for example, a PCSK9 Arg46Leu polypeptide.

In any of the embodiments described herein, the subject can have sepsis, severe sepsis, SIRS, septic shock, and/or MODS. In any of the embodiments described herein, the subject can have sepsis. In any of the embodiments described herein, the subject can have severe sepsis. In any of the embodiments described herein, the subject can have SIRS. In any of the embodiments described herein, the subject can have septic shock. In any of the embodiments described herein, the subject can have MODS.

Symptoms of sepsis include, but are not limited to, fever, diarrhea, a decrease in blood pressure, leaky blood vessels, and/or disseminated blood clotting in a variety of organs. The underlying dysfunctions include, but are not limited to, arterial hypotension, metabolic acidosis, decreased systemic vascular resistance, tachypnea, organ dysfunction, and septicemia.

Symptoms of SIRS include, but are not limited to, any two or more of the following conditions: 1) body temperature >38° C. or <36° C.; 2) heart rate >90 beats/minute; 3) frequency of respiration >20 breaths/minute or PaCO₂<32 mmHg; and 4) number of leukocytes >12,000/μl or <4000/μl, or ratio of stab neutrophil >10%.

Symptoms of septic shock include, but are not limited to, a decrease in blood pressure to dangerous levels in a subject having sepsis.

Symptoms of MODS include, but are not limited to, failure of two or more vital organ systems.

The present disclosure provides methods of treating a subject having sepsis, SIRS, septic shock, and/or MODS, the methods comprising administering a PCSK9 inhibitor and/or an LDLR agonist to the subject.

The present disclosure also provides methods of treating a subject having sepsis, the methods comprising administering a PCSK9 inhibitor and/or an LDLR agonist to the subject.

The present disclosure also provides methods of treating a subject having SIRS, the methods comprising administering a PCSK9 inhibitor and/or an LDLR agonist to the subject.

The present disclosure also provides methods of treating a subject having septic shock, the methods comprising administering a PCSK9 inhibitor and/or an LDLR agonist to the subject.

The present disclosure also provides methods of treating a subject having MODS, the methods comprising administering a PCSK9 inhibitor and/or an LDLR agonist to the subject.

In some embodiments, the PCSK9 inhibitor and/or LDLR agonist comprise an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such antisense molecules can be designed to target any region of a PCSK9 and/or an LDLR nucleic acid molecule, such as an mRNA molecule. In some embodiments, the antisense molecule, siRNA, or shRNA hybridizes to a sequence within a PCSK9 and/or an LDLR genomic nucleic acid molecule or mRNA molecule and decreases expression of the PCSK9 and/or LDLR polypeptide in a cell in the subject. In some embodiments, the PCSK9 inhibitor and/or LDLR agonist comprise an antisense molecule that hybridizes to a PCSK9 and/or an LDLR genomic nucleic acid molecule or mRNA molecule and decreases expression of the PCSK9 and/or LDLR polypeptide in a cell in the subject. In some embodiments, the PCSK9 inhibitor and/or LDLR agonist comprise an siRNA that hybridizes to a PCSK9 and/or an LDLR genomic nucleic acid molecule or mRNA molecule and decreases expression of the PCSK9 and/or LDLR polypeptide in a cell in the subject. In some embodiments, the PCSK9 inhibitor and/or LDLR agonist comprise an shRNA that hybridizes to a PCSK9 and/or an LDLR genomic nucleic acid molecule or mRNA molecule and decreases expression of the PCSK9 and/or LDLR polypeptide in a cell in the subject.

In some embodiments, the PCSK9 inhibitor and/or LDLR agonist comprise a nuclease agent that induces one or more nicks or double-strand breaks at a recognition sequence(s) or a DNA-binding protein that binds to a recognition sequence within a PCSK9 genomic nucleic acid molecule and/or an LDLR genomic nucleic acid molecule. The recognition sequence can be located within a coding region of the PCSK9 gene and/or LDLR gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the PCSK9 gene and/or LDLR gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a PCSK9 genomic nucleic acid molecule and/or an LDLR genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of PCSK9 nucleic acid molecules and/or LDLR nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a PCSK9 genomic nucleic acid molecule and/or an LDLR genomic nucleic acid molecule or it can be a nickase that creates a single-strand break in a PCSK9 genomic nucleic acid molecule and/or an LDLR genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of the PCSK9 genomic nucleic acid molecule and/or the LDLR genomic nucleic acid molecule can be generated by contacting a cell with a Cas protein and one or more gRNAs that hybridize to one or more gRNA recognition sequences within a target genomic locus in the PCSK9 genomic nucleic acid molecule and/or the LDLR genomic nucleic acid molecule. For example, a gRNA recognition sequence can be located within a region of SEQ ID NO:1 and/or SEQ ID NO:3. The gRNA recognition sequence can also include or be proximate to a position corresponding to: position 427 according to SEQ ID NO:1, or position 2,269 according to SEQ ID NO: 3. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 427 according to SEQ ID NO:1, or position 2,269 according to SEQ ID NO: 3. The gRNA recognition sequence can include or be proximate to the start codon of the PCSK9 genomic nucleic acid molecule and/or the LDLR genomic nucleic acid molecule or the stop codon of the PCSK9 genomic nucleic acid molecule and/or the LDLR genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in the PCSK9 genomic nucleic acid molecule and/or the LDLR genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-31, where N is any DNA nucleotide and is immediately 5′ of the gRNA recognition sequence of the complementary strand of the target DNA.

A gRNA is an RNA molecule that binds to a Cas protein and targets the Cas protein to a specific location within the PCSK9 genomic nucleic acid molecule and/or the LDLR genomic nucleic acid molecule. An exemplary gRNA is a gRNA effective to direct a Cas enzyme to bind to or cleave the PCSK9 genomic nucleic acid molecule and/or the LDLR genomic nucleic acid molecule, wherein the gRNA comprises a DNA-targeting segment that hybridizes to a gRNA recognition sequence within the PCSK9 genomic nucleic acid molecule and/or the LDLR genomic nucleic acid molecule that includes or is proximate to a position corresponding to: position 427 according to SEQ ID NO:1, or position 2,269 according to SEQ ID NO: 3. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of a position corresponding to: position 427 according to SEQ ID NO: 1, or position 2,269 according to SEQ ID NO:3. Other exemplary gRNAs comprise a DNA-targeting segment that hybridizes to a gRNA recognition sequence present within the PCSK9 genomic nucleic acid molecule and/or the LDLR genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the PCSK9 reference gene are set forth in Table 1 as SEQ ID NOS: 43-68. Examples of suitable gRNA recognition sequences located within the LDLR reference gene are set forth in Table 1 as SEQ ID NOS: 69-89.

TABLE 1
Guide RNA Recognition Sequences Near the
PCSK9 and/or LDLR Variation(s)

| | PCSK9 gRNA | |
|---|---|---|
| Strand | Recognition Sequence | SEQ ID NO: |
| − | CCCGGTCCTTGGCGCGCGGT | 43 |
| + | AGGACGAGGACGGCGACACG | 44 |
| − | CCGTGCTCGGGTGCTTCGGC | 45 |
| − | CCGGTCCTTGGCGCGCGGTG | 46 |
| − | CTTCGGCCGGCCGTCCTCCT | 47 |
| + | AGCCACCCCACCGCGCGCCA | 48 |
| − | GGTTCCGTGCTCGGGTGCTT | 49 |
| − | TCCCGGTCCTTGGCGCGCGG | 50 |
| + | GAGGACGGCGACACGAGGAG | 51 |
| + | ACCGCGCGCCAAGGACCGGG | 52 |
| + | CCGCGCGCCAAGGACCGGGA | 53 |
| + | CCCACCGCGCGCCAAGGACC | 54 |
| − | GCCGGCCGTCCTCCTCGGCG | 55 |
| − | GGTGGCTGTGGTTCCGTGCT | 56 |
| − | CCCTCCCGGTCCTTGGCGCG | 57 |
| + | AGGACGGCGACACGAGGAGC | 58 |
| + | ACCGGGAGGGCCGGCACCAC | 59 |
| − | GTGGCTGTGGTTCCGTGCTC | 60 |
| + | CCCCACCGCGCGCCAAGGAC | 61 |
| − | GTGCCGGCCCTCCCGGTCCT | 62 |
| + | AGGAGCGGGCAGCCGCGCCG | 63 |
| + | CGGGAGGGCCGGCACCACGG | 64 |
| + | AGCGGGCAGCCGCGCCGAGG | 65 |
| + | CCGGGAGGGCCGGCACCACG | 66 |
| + | GACCGGGAGGGCCGGCACCA | 67 |
| − | GTCCTTGGCGCGCGGTGGGG | 68 |
| | LDLR gRNA | |
| Strand | Recognition Sequence | SEQ ID NO: |
| − | GTCATAGGAAGAGACGCCGT | 69 |
| + | CGGATACCAAGGGCGTGAAG | 70 |
| − | TGTCATAGGAAGAGACGCCG | 71 |
| + | CGTGGTCGCTCTGGACACGG | 72 |
| + | TGTCTCTGTTGCGGATACCA | 73 |
| − | GTCCACAGCCAGCCCGTCGG | 74 |
| − | AGACCAGTAGATTCTATTGC | 75 |
| − | CGGTCCAGTAGATGTTGCTG | 76 |
| − | CGCCGTGGGCTCTGTCAAGC | 77 |
| + | GAACGTGGTCGCTCTGGACA | 78 |
| + | CTACTGGACCGACTCTGTCC | 79 |
| + | AGAGACATCCAGGCCCCCGA | 80 |
| − | TCTGCTGATGACGGTGTCAT | 81 |
| + | GAGACATCCAGGCCCCCGAC | 82 |
| − | GCCGTGGGCTCTGTCAAGCT | 83 |
| + | GTCTCTGTTGCGGATACCAA | 84 |
| + | GGTCGCTCTGGACACGGAGG | 85 |
| + | TGGCCAGCAATAGAATCTAC | 86 |
| + | CATCCAGGCCCCCGACGGGC | 87 |
| − | GGGTGCTGCAGATCATTCTC | 88 |
| + | TACTGGACCGACTCTGTCCT | 89 |

The Cas protein and the gRNA form a complex, and the Cas protein cleaves the target PCSK9 genomic nucleic acid molecule and/or target LDLR genomic nucleic acid molecule. The Cas protein can cleave the nucleic acid molecule at a site within or outside of the nucleic acid sequence present in the target PCSK9 genomic nucleic acid molecule and/or LDLR genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the PCSK9 genomic nucleic acid molecule and/or LDLR genomic nucleic acid molecule to which a DNA-targeting segment of a gRNA will bind.

Such methods can result, for example, in a PCSK9 genomic nucleic acid molecule and/or an LDLR genomic nucleic acid molecule in which a region of SEQ ID NO:1 or SEQ ID NO: 3, respectively, is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the PCSK9 genomic nucleic acid molecule and/or the LDLR genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the PCSK9 inhibitor comprises a small molecule or an antibody. In some embodiments, the PCSK9 inhibitor is alirocumab or evolocumab, or a combination thereof. In some embodiments, the PCSK9 inhibitor is the monoclonal antibody SAR236553/REGN727 or AMG145, or a combination thereof. In some embodiments, the PCSK9 inhibitor is lupin peptide, resveratrol, lycopene, or eugenol, or any combination thereof.

In some embodiments, the LDLR agonist comprises a small molecule or an antibody. In some embodiments, the LDLR agonist is one of the PCSK9 inhibitors described herein. In some embodiments, the LDLR agonist is lupin peptide, resveratrol, lycopene, suramin, or puromycin, or any combination thereof.

In some embodiments, the methods of treatment further comprise detecting the presence or absence of a PCSK9 variant nucleic acid molecule and/or an LDLR variant nucleic acid molecule in a biological sample from the subject. As used throughout the present disclosure, a “PCSK9 variant nucleic acid molecule” is any PCSK9 nucleic acid molecule (such as, for example, genomic nucleic acid molecule, mRNA molecule, or cDNA molecule) encoding a PCSK9 polypeptide having a partial loss-of-function, a complete loss-of-function, a predicted partial loss-of-function, or a predicted complete loss-of-function.

The present disclosure also provides methods of treating a subject with a therapeutic agent that treats or inhibits sepsis, SIRS, septic shock, and/or MODS. In some embodiments, the subject is suffering from sepsis, SIRS, septic shock, and/or MODS. In some embodiments, the subject is suffering from sepsis. In some embodiments, the subject is suffering from SIRS. In some embodiments, the subject is suffering from septic shock. In some embodiments, the subject is suffering from MODS. In some embodiments, the methods comprise determining whether the subject has a PCSK9 variant nucleic acid molecule and/or an LDLR variant nucleic acid molecule by obtaining or having obtained a biological sample from the subject, and performing or having performed a sequence analysis on the biological sample to determine if the subject has a genotype comprising the PCSK9 variant nucleic acid molecule and/or the LDLR variant nucleic acid molecule.

In some embodiments, when the subject is PCSK9 reference and LDLR reference, the therapeutic agent that treats or inhibits sepsis, SIRS, septic shock, and/or MODS is administered or continued to be administered to the subject in a standard dosage amount, and a PCSK9 inhibitor and/or the LDLR agonist is administered to the subject.

In some embodiments, when the subject is LDLR reference and heterozygous for a PCSK9 variant nucleic acid molecule, the therapeutic agent that treats or inhibits sepsis, SIRS, septic shock, and/or MODS is administered or continued to be administered in an amount that is the same as or less than a standard dosage amount, and the PCSK9 inhibitor and/or the LDLR agonist is administered to the subject.

In some embodiments, when the subject is PCSK9 reference and heterozygous for an LDLR variant nucleic acid molecule, the therapeutic agent that treats or inhibits sepsis, SIRS, septic shock, and/or MODS is administered or continued to be administered in an amount that is the same as or less than a standard dosage amount, and the PCSK9 inhibitor and/or the LDLR agonist is administered to the subject.

In some embodiments, when the subject is LDLR reference and homozygous for a PCSK9 variant nucleic acid molecule, the therapeutic agent that treats or inhibits sepsis, SIRS, septic shock, and/or MODS is administered or continued to be administered in an amount that is the same as or less than a standard dosage amount, and an LDLR agonist is administered to the subject.

In some embodiments, when the subject is PCSK9 reference and homozygous for an LDLR variant nucleic acid molecule, the therapeutic agent that treats or inhibits sepsis, SIRS, septic shock, and/or MODS is administered or continued to be administered in an amount that is the same as or less than a standard dosage amount, and a PCSK9 inhibitor is administered to the subject.

In some embodiments, when the subject is heterozygous for both a PCSK9 variant nucleic acid molecule and an LDLR variant nucleic acid molecule, the therapeutic agent that treats or inhibits sepsis, SIRS, septic shock, and/or MODS is administered or continued to be administered in an amount that is the same as or less than a standard dosage amount, and the PCSK9 inhibitor and/or the LDLR agonist is administered to the subject.

In some embodiments, when the subject is homozygous for both a PCSK9 variant nucleic acid molecule and an LDLR variant nucleic acid molecule, the therapeutic agent that treats or inhibits sepsis, SIRS, septic shock, and/or MODS is administered or continued to be administered in a dosage amount that is the same as or less than a standard dosage amount.

The presence of a genotype having the PCSK9 variant nucleic acid molecule and/or the LDLR variant nucleic acid molecule indicates the subject has a decreased risk of developing sepsis, SIRS, septic shock, and/or MODS. In some embodiments, the subject is PCSK9 reference and LDLR reference. In some embodiments, the subject is heterozygous or homozygous for a PCSK9 variant nucleic acid molecule and LDLR reference. In some embodiments, the subject is heterozygous or homozygous for an LDLR variant nucleic acid molecule and PCSK9 reference. In some embodiments, the subject is heterozygous or homozygous for an LDLR variant nucleic acid molecule and PCSK9 heterozygous or homozygous for a PCSK9 variant nucleic acid molecule.

Detecting the presence or absence of a PCSK9 variant nucleic acid molecule and/or an LDLR variant nucleic acid molecule in a biological sample from a subject and/or determining whether a subject has a PCSK9 variant nucleic acid molecule and/or an LDLR variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is PCSK9 reference and/or LDLR reference, the subject is also administered a therapeutic agent that treats or inhibits sepsis, SIRS, septic shock, and/or MODS in a standard dosage amount. In some embodiments, when the subject is heterozygous or homozygous for a PCSK9 variant nucleic acid molecule and/or an LDLR variant nucleic acid molecule, the subject is also administered a therapeutic agent that treats or inhibits sepsis, SIRS, septic shock, and/or MODS in a dosage amount that is the same as or less than a standard dosage amount.

In some embodiments, the treatment methods further comprise detecting the presence or absence of a PCSK9 predicted loss-of-function polypeptide in a biological sample from the subject. In some embodiments, when the subject does not have a PCSK9 predicted loss-of-function polypeptide, the subject is also administered a therapeutic agent that treats or inhibits sepsis, SIRS, septic shock, and/or MODS in a standard dosage amount. In some embodiments, when the subject has a PCSK9 predicted loss-of-function polypeptide, the subject is also administered a therapeutic agent that treats or inhibits sepsis, SIRS, septic shock, and/or MODS in a dosage amount that is the same as or less than a standard dosage amount.

The present disclosure also provides methods of treating a subject with a therapeutic agent that treats or inhibits sepsis, SIRS, septic shock, and/or MODS. In some embodiments, the subject is suffering from sepsis, SIRS, septic shock, and/or MODS. In some embodiments, the subject is suffering from sepsis. In some embodiments, the subject is suffering from SIRS. In some embodiments, the subject is suffering from septic shock. In some embodiments, the subject is suffering from MODS. In some embodiments, the method comprises determining whether the subject has a PCSK9 predicted loss-of-function polypeptide by obtaining or having obtained a biological sample from the subject, and performing or having performed an assay on the biological sample to determine if the subject has a PCSK9 predicted loss-of-function polypeptide. When the subject does not have a PCSK9 predicted loss-of-function polypeptide, the therapeutic agent that treats or inhibits sepsis, SIRS, septic shock, and/or MODS is administered or continued to be administered to the subject in a standard dosage amount, and a PCSK9 inhibitor and/or an LDLR agonist is administered to the subject. When the subject has a PCSK9 predicted loss-of-function polypeptide, the therapeutic agent that treats or inhibits sepsis, SIRS, septic shock, and/or MODS is administered or continued to be administered to the subject in an amount that is the same as or less than a standard dosage amount, and a PCSK9 inhibitor and/or an LDLR agonist is administered to the subject. The presence of a PCSK9 predicted loss-of-function polypeptide indicates the subject has a decreased risk of developing sepsis, SIRS, septic shock, and/or MODS. In some embodiments, the subject has a PCSK9 predicted loss-of-function polypeptide. In some embodiments, the subject does not have a PCSK9 predicted loss-of-function polypeptide.

Detecting the presence or absence of a PCSK9 predicted loss-of-function polypeptide in a biological sample from a subject and/or determining whether a subject has a PCSK9 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents and therapies that treat or inhibit sepsis, severe sepsis, SIRS, septic shock, and/or MODS can depend on the affected organ(s), the severity of the condition, the time of diagnosis, and underlying cause(s) (e.g. infection, trauma, etc.). Early treatment includes antibiotics and intravenous fluids, as well as high-flow oxygen. Treatment can also include administering red blood cells, vasopressors (e.g., norepinephrine in case of hypotensive septic shock), and, in some cases, steroids. Surgical interventions may be used to control the infection source (e.g., drainage of pus from an abscess). Further sepsis management includes monitoring organ function to identify the organs and systems affected. Additional therapies may then be implemented to specifically target the affected organs. In the case of particularly severe sepsis that results in MODS, therapy is usually mostly limited to supportive care (i.e., safeguarding hemodynamics), and respiration. In addition, several classes of therapeutics are used in sepsis management in the clinic, including recombinant activated protein C, TLR4 antagonists, C5a antagonists, C1 inhibitors, endotoxin removal devices, caspase inhibitors, Oestrogen receptor-β, and statins, among others (reviewed in Shukla et al., Br. J. Pharmacol., 2014, 171, 5011-5031).

In some embodiments, the dose of the therapeutic agents that treat or inhibit sepsis, SIRS, septic shock, and/or MODS can be reduced by about 10%, by about 20%, by about 30%, by about 40%, by about 50%, by about 60%, by about 70%, by about 80%, or by about 90% for subjects that are heterozygous for a PCSK9 variant nucleic acid molecule and/or an LDLR variant nucleic acid molecule (i.e., less than the standard dosage amount) compared to subjects that are PCSK9 reference and/or LDLR reference (who may receive a standard dosage amount). In some embodiments, the dose of the therapeutic agents that treat or inhibit sepsis, SIRS, septic shock, and/or MODS can be reduced by about 10%, by about 20%, by about 30%, by about 40%, or by about 50%. In addition, the dose of therapeutic agents that treat or inhibit sepsis, SIRS, septic shock, and/or MODS in subjects that are heterozygous for a PCSK9 variant nucleic acid molecule and/or an LDLR variant nucleic acid molecule can be administered less frequently compared to subjects that are PCSK9 reference and/or LDLR reference.

Administration of the therapeutic agents that treat or inhibit sepsis, SIRS, septic shock, and/or MODS and/or a PCSK9 inhibitor and/or an LDLR agonist can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit sepsis, SIRS, septic shock, and/or MODS and/or a PCSK9 inhibitor and/or an LDLR agonist can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in sepsis, SIRS, septic shock, and/or MODS (i.e., condition), a decrease/reduction in the severity of sepsis, SIRS, septic shock, and/or MODS (such as, for example, a reduction or inhibition of development or sepsis, SIRS, septic shock, and/or MODS), a decrease/reduction in symptoms and condition-related effects, delaying the onset of symptoms and condition-related effects, reducing the severity of symptoms of condition-related effects, reducing the severity of an acute episode, reducing the number of symptoms and condition-related effects, reducing the latency of symptoms and condition-related effects, an amelioration of symptoms and condition-related effects, reducing secondary symptoms, reducing secondary infections, preventing relapse to sepsis, SIRS, septic shock, and/or MODS, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of sepsis, SIRS, septic shock, and/or MODS development/progression (such as, for example, a complete or partial avoidance/inhibition or a delay), and an increased survival time of the affected host animal, following administration of a therapeutic protocol. Treatment of sepsis, SIRS, septic shock, and/or MODS encompasses the treatment of subjects already diagnosed as having any form of sepsis, SIRS, septic shock, and/or MODS at any clinical stage or manifestation, the delay of the onset or evolution or aggravation or deterioration of the symptoms or signs of sepsis, SIRS, septic shock, and/or MODS, and/or preventing and/or reducing the severity of sepsis, SIRS, septic shock, and/or MODS.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof.

The present disclosure also provides methods of identifying a subject having an increased risk of developing sepsis, SIRS, septic shock, and/or MODS. In some embodiments, the methods identify a subject having an increased risk of developing sepsis. In some embodiments, the methods identify a subject having an increased risk of developing SIRS. In some embodiments, the methods identify a subject having an increased risk of developing septic shock. In some embodiments, the methods identify a subject having an increased risk of developing MODS. In some embodiments, the methods comprise determining or having determined in a biological sample obtained from the subject the presence or absence of a PCSK9 variant nucleic acid molecule and/or an LDLR variant nucleic acid molecule (such as a genomic nucleic acid molecule, mRNA molecule, and/or cDNA molecule). When the subject lacks a PCSK9 variant nucleic acid molecule and an LDLR variant nucleic acid molecule (i.e., the subject is genotypically categorized as PCSK9 reference and LDLR reference), then the subject has an increased risk of developing sepsis, SIRS, septic shock, and/or MODS. When the subject has a PCSK9 variant nucleic acid molecule and/or an LDLR variant nucleic acid molecule (i.e., the subject is heterozygous or homozygous for a PCSK9 variant nucleic acid molecule and/or an LDLR variant nucleic acid molecule), then the subject has a decreased risk of developing sepsis, SIRS, septic shock, and/or MODS.

Having a single copy of a PCSK9 variant nucleic acid molecule and/or an LDLR variant nucleic acid molecule is more protective of a subject from developing sepsis, SIRS, septic shock, and/or MODS than having no copies of a PCSK9 variant nucleic acid molecule and/or an LDLR variant nucleic acid molecule. Without intending to be limited to any particular theory or mechanism of action, it is believed that a single copy of a PCSK9 variant nucleic acid molecule and/or an LDLR variant nucleic acid molecule (i.e., heterozygous for a PCSK9 variant nucleic acid molecule and/or an LDLR variant nucleic acid molecule) is protective of a subject from developing sepsis, SIRS, septic shock, and/or MODS, and it is also believed that having two copies of a PCSK9 variant nucleic acid molecule and/or an LDLR variant nucleic acid molecule (i.e., homozygous for a PCSK9 variant predicted loss-of-function and/or an LDLR variant predicted loss-of-function) may be more protective of a subject from developing sepsis, SIRS, septic shock, and/or MODS, relative to a subject with a single copy. Thus, in some embodiments, a single copy of a PCSK9 variant nucleic acid molecule and/or an LDLR variant nucleic acid molecule may not be completely protective, but instead, may be partially or incompletely protective of a subject from developing sepsis, SIRS, septic shock, and/or MODS. While not desiring to be bound by any particular theory, there may be additional factors or molecules involved in the development of sepsis, SIRS, septic shock, and/or MODS that are still present in a subject having a single copy of a PCSK9 variant nucleic acid molecule and/or an LDLR variant nucleic acid molecule, thus resulting in less than complete protection from the development of sepsis, SIRS, septic shock, and/or MODS.

Determining whether a subject has a PCSK9 variant nucleic acid molecule and/or an LDLR variant nucleic acid molecule in a biological sample from a subject and/or determining whether a subject has a PCSK9 variant nucleic acid molecule and/or an LDLR variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing sepsis, SIRS, septic shock, and/or MODS, the subject is further treated with a therapeutic agent that treats or inhibits sepsis, SIRS, septic shock, and/or MODS and/or a PCSK9 inhibitor and/or an LDLR agonist, as described herein. For example, when the subject is PCSK9 reference and/or LDLR reference, and therefore has an increased risk of developing sepsis, SIRS, septic shock, and/or MODS, the subject is administered a PCSK9 inhibitor and/or an LDLR agonist. In some embodiments, such a subject is also administered a therapeutic agent that treats or inhibits sepsis, SIRS, septic shock, and/or MODS. In some embodiments, when the subject is heterozygous for a PCSK9 variant nucleic acid molecule and/or an LDLR variant nucleic acid molecule, the subject is administered the therapeutic agent that treats or inhibits sepsis, SIRS, septic shock, and/or MODS in a dosage amount that is the same as or less than a standard dosage amount, and is also administered a PCSK9 inhibitor and/or an LDLR agonist.

In some embodiments, the subject is PCSK9 reference and LDLR reference. In some embodiments, the subject is heterozygous or homozygous for a PCSK9 variant nucleic acid molecule and LDLR reference. In some embodiments, the subject is heterozygous or homozygous for an LDLR variant nucleic acid molecule and PCSK9 reference. In some embodiments, the subject is heterozygous or homozygous for an LDLR variant nucleic acid molecule and PCSK9 heterozygous or homozygous for a PCSK9 variant nucleic acid molecule.

The present disclosure also provides methods of detecting the presence or absence of a PCSK9 variant genomic nucleic acid molecule and/or an LDLR variant genomic nucleic acid molecule in a biological sample from a subject, and/or a PCSK9 variant mRNA molecule in a biological sample from a subject, and/or a PCSK9 variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the PCSK9 and/or LDLR variant genomic nucleic acid molecule, the PCSK9 variant mRNA molecules, and the PCSK9 variant cDNA molecules are only exemplary sequences. Other sequences for the PCSK9 and/or LDLR variant genomic nucleic acid molecule, variant mRNA molecules, and variant cDNA molecules are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any PCSK9 and/or any LDLR variant nucleic acid molecule, preliminary processing designed to isolate or enrich the biological sample for the genomic DNA can be employed. A variety of techniques may be used for this purpose. When detecting the level of any PCSK9 and/or any LDLR variant mRNA molecule, different techniques can be used enrich the biological sample with mRNA molecules. Various methods to detect the presence or level of an mRNA molecule or the presence of a particular variant genomic DNA locus can be used.

In some embodiments, detecting a PCSK9 variant nucleic acid molecule and/or LDLR variant nucleic acid molecule in a subject comprises assaying or genotyping a biological sample obtained from the subject to determine whether a PCSK9 genomic nucleic acid molecule and/or an LDLR genomic nucleic acid molecule in the biological sample, and/or a PCSK9 mRNA molecule in the biological sample, and/or a PCSK9 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of a PCSK9 variant nucleic acid molecule and/or an LDLR variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the PCSK9 nucleotide sequence comprises: a thymine at a position corresponding to position 427 according to SEQ ID NO: 2 (for genomic nucleic acid molecules); a uracil at a position corresponding to position 428 according to SEQ ID NO:17, a uracil at a position corresponding to position 217 according to SEQ ID NO:18, or a uracil at a position corresponding to position 137 according to SEQ ID NO:19 (for mRNA molecules); or a thymine at a position corresponding to position 428 according to SEQ ID NO:32, a thymine at a position corresponding to position 217 according to SEQ ID NO:33, or a thymine at a position corresponding to position 137 according to SEQ ID NO:34 (for cDNA molecules obtained from mRNA molecules).

In some embodiments, the PCSK9 genomic nucleotide sequence comprises a thymine at a position corresponding to position 427 according to SEQ ID NO:2, or the complement thereof.

In some embodiments, the PCSK9 mRNA nucleotide sequence comprises a uracil at a position corresponding to: position 428 according to SEQ ID NO:17, or the complement thereof; position 217 according to SEQ ID NO:18, or the complement thereof; or position 137 according to SEQ ID NO:19, or the complement thereof.

In some embodiments, the PCSK9 cDNA nucleotide sequence comprises a thymine at a position corresponding to: position 428 according to SEQ ID NO:32, or the complement thereof; position 217 according to SEQ ID NO:33, or the complement thereof; or position 137 according to SEQ ID NO:34, or the complement thereof.

In some embodiments, the LDLR nucleotide sequence comprises a thymine at a position corresponding to position 2,269 according to SEQ ID NO: 4 (for genomic nucleic acid molecules). In some embodiments, the LDLR genomic nucleotide sequence comprises a thymine at a position corresponding to position 2,269 according to SEQ ID NO:4, or the complement thereof.

In some embodiments, the biological sample comprises a cell or cell lysate. Such methods can further comprise, for example, obtaining a biological sample from the subject comprising a PCSK9 genomic nucleic acid molecule or mRNA molecule and/or an LDLR genomic nucleic acid molecule, and if mRNA, optionally reverse transcribing the mRNA into cDNA. Such assays can comprise, for example determining the identity of these positions of the particular a PCSK9 nucleic acid molecule and/or an LDLR nucleic acid molecule. In some embodiments, the method is an in vitro method.

In some embodiments, the determining step, detecting step, or sequence analysis comprises sequencing at least a portion of the nucleotide sequence of a PCSK9 genomic nucleic acid molecule and/or an LDLR genomic nucleic acid molecule, a PCSK9 mRNA molecule, or a PCSK9 cDNA molecule produced from an mRNA molecule in the biological sample, wherein the sequenced portion comprises one or more variations described herein.

In some embodiments, the determining step, detecting step, or sequence analysis comprises sequencing at least a portion of: i) the nucleotide sequence of the PCSK9 and/or the LDLR genomic nucleic acid molecule in the biological sample, wherein the sequenced portion comprises: a position corresponding to position 427 according to SEQ ID NO:2, or the complement thereof; or a position corresponding to position 2,269 according to SEQ ID NO: 4, or the complement thereof; ii) the nucleotide sequence of the PCSK9 mRNA molecule in the biological sample, wherein the sequenced portion comprises a position corresponding to: position 428 according to SEQ ID NO:17, or the complement thereof; position 217 according to SEQ ID NO:18, or the complement thereof; or position 137 according to SEQ ID NO: 19, or the complement thereof; and/or iii) the nucleotide sequence of the PCSK9 cDNA molecule produced from the mRNA in the biological sample, wherein the sequenced portion comprises a position corresponding to: position 428 according to SEQ ID NO:32, or the complement thereof; position 217 according to SEQ ID NO:33, or the complement thereof; or position 137 according to SEQ ID NO:34, or the complement thereof. When the sequenced portion of the PCSK9 genomic nucleic acid molecule and/or the LDLR genomic nucleic acid molecule in the biological sample comprise: a thymine at a position corresponding to position 427 according to SEQ ID NO:2, or a thymine at a position corresponding to position 2,269 according to SEQ ID NO:4, respectively; or when the sequenced portion of the PCSK9 mRNA molecule in the biological sample comprises a uracil at a position corresponding to: position 428 according to SEQ ID NO:17, position 217 according to SEQ ID NO:18, or position 137 according to SEQ ID NO:19; or when the sequenced portion of the PCSK9 cDNA molecule in the biological sample comprises a thymine at a position corresponding to: position 428 according to SEQ ID NO:32, position 217 according to SEQ ID NO:33, or position 137 according to SEQ ID NO:34; then the PCSK9 nucleic acid molecule and/or the LDLR nucleic acid molecule in the biological sample is a PCSK9 variant nucleic acid molecule and/or an LDLR variant nucleic acid molecule.

In some embodiments, the determining step, detecting step, or sequence analysis comprises sequencing at least a portion of the nucleotide sequence of the PCSK9 genomic nucleic acid molecule and/or the LDLR genomic nucleic acid molecule in the biological sample, wherein the sequenced portion comprises: a position corresponding to position 427 according to SEQ ID NO:2, or the complement thereof; or a position corresponding to position 2,269 according to SEQ ID NO:4, or the complement thereof. When the sequenced portion of the PCSK9 nucleic acid molecule and/or the LDLR nucleic acid molecule in the biological sample comprises: a thymine at a position corresponding to position 427 according to SEQ ID NO:2, or a thymine at a position corresponding to position 2,269 according to SEQ ID NO:4, respectively, then the PCSK9 nucleic acid molecule and/or the LDLR nucleic acid molecule in the biological sample is a PCSK9 variant nucleic acid molecule and/or an LDLR variant nucleic acid molecule.

In some embodiments, the determining step, detecting step, or sequence analysis comprises sequencing at least a portion of the nucleotide sequence of the PCSK9 mRNA molecule in the biological sample, wherein the sequenced portion comprises a position corresponding to: position 428 according to SEQ ID NO:17, or the complement thereof; position 217 according to SEQ ID NO:18, or the complement thereof; or position 137 according to SEQ ID NO:19, or the complement thereof. When the sequenced portion of the PCSK9 nucleic acid molecule in the biological sample comprises a uracil at a position corresponding to: position 428 according to SEQ ID NO:17, position 217 according to SEQ ID NO: 18, or position 137 according to SEQ ID NO:19, then the PCSK9 nucleic acid molecule in the biological sample is a PCSK9 variant nucleic acid molecule.

In some embodiments, the determining step, detecting step, or sequence analysis comprises sequencing at least a portion of the nucleotide sequence of the PCSK9 cDNA molecule produced from the mRNA molecule in the biological sample, wherein the sequenced portion comprises a position corresponding to: position 428 according to SEQ ID NO: 32, or the complement thereof; position 217 according to SEQ ID NO:33, or the complement thereof; or position 137 according to SEQ ID NO:34, or the complement thereof. When the sequenced portion of the PCSK9 nucleic acid molecule in the biological sample comprises a thymine at a position corresponding to: position 428 according to SEQ ID NO: 32, position 217 according to SEQ ID NO:33, or position 137 according to SEQ ID NO: 34, then the PCSK9 nucleic acid molecule in the biological sample is a PCSK9 variant nucleic acid molecule.

In some embodiments, the determining step, detecting step, or sequence analysis comprises: a) contacting the biological sample with a primer hybridizing to a portion of the nucleotide sequence of: a PCSK9 genomic nucleic acid molecule that is proximate to a position corresponding to position 427 according to SEQ ID NO:2, or an LDLR genomic nucleic acid molecule that is proximate to a position corresponding to position 2,269 according to SEQ ID NO:4; a PCSK9 mRNA molecule that is proximate to a position corresponding to: position 428 according to SEQ ID NO:17, position 217 according to SEQ ID NO: 18, or position 137 according to SEQ ID NO:19; and/or a PCSK9 cDNA molecule that is proximate to a position corresponding to: position 428 according to SEQ ID NO:32, position 217 according to SEQ ID NO:33, or position 137 according to SEQ ID NO:34; b) extending the primer at least through the position of the nucleotide sequence of: the PCSK9 genomic nucleic acid molecule corresponding to position 427 according to SEQ ID NO:2, or the LDLR genomic nucleic acid molecule corresponding to position 2,269 according to SEQ ID NO:4; the PCSK9 mRNA molecule corresponding to: position 428 according to SEQ ID NO:17, position 217 according to SEQ ID NO:18, or position 137 according to SEQ ID NO:19; and/or the PCSK9 cDNA molecule corresponding to: position 428 according to SEQ ID NO:32, position 217 according to SEQ ID NO:33, or position 137 according to SEQ ID NO:34; and c) determining whether the extension product of the primer comprises: i) a thymine at a position corresponding to position 427 according to SEQ ID NO:2, or a thymine at a position corresponding to position 2,269 according to SEQ ID NO:4; ii) a uracil at a position corresponding to: position 428 according to SEQ ID NO:17, position 217 according to SEQ ID NO: 18, or position 137 according to SEQ ID NO: 19; and/or iii) a thymine at a position corresponding to: position 428 according to SEQ ID NO:32, position 217 according to SEQ ID NO: 33, or position 137 according to SEQ ID NO:34.

In some embodiments, the determining step, detecting step, or sequence analysis comprises: a) contacting the biological sample with a primer hybridizing to a portion of the nucleotide sequence of the PCSK9 genomic nucleic acid molecule and/or the LDLR genomic nucleic acid molecule that is proximate to a position corresponding to: position 427 according to SEQ ID NO:2, or position 2,269 according to SEQ ID NO:4, respectively; b) extending the primer at least through the position of the nucleotide sequence of the PCSK9 genomic nucleic acid molecule and/or the LDLR genomic nucleic acid molecule corresponding to: position 427 according to SEQ ID NO:2, or position 2,269 according to SEQ ID NO: 4, respectively; and c) determining whether the extension product of the primer comprises: a thymine at a position corresponding to position 427 according to SEQ ID NO:2, or a thymine at a position corresponding to position 2,269 according to SEQ ID NO:4.

In some embodiments, the determining step, detecting step, or sequence analysis comprises: a) contacting the biological sample with a primer hybridizing to a portion of the nucleotide sequence of the PCSK9 mRNA molecule that is proximate to a position corresponding to: position 428 according to SEQ ID NO:17, position 217 according to SEQ ID NO: 18, or position 137 according to SEQ ID NO:19; b) extending the primer at least through the position of the nucleotide sequence of the PCSK9 mRNA molecule corresponding to: position 428 according to SEQ ID NO:17, position 217 according to SEQ ID NO:18, or position 137 according to SEQ ID NO:19; and c) determining whether the extension product of the primer comprises a uracil at a position corresponding to: position 428 according to SEQ ID NO: 17, position 217 according to SEQ ID NO:18, or position 137 according to SEQ ID NO:19.

In some embodiments, the determining step, detecting step, or sequence analysis comprises: a) contacting the biological sample with a primer hybridizing to a portion of the nucleotide sequence of the PCSK9 cDNA molecule that is proximate to a position corresponding to: position 428 according to SEQ ID NO:32, position 217 according to SEQ ID NO: 33, or position 137 according to SEQ ID NO:34; b) extending the primer at least through the position of the nucleotide sequence of the PCSK9 cDNA molecule corresponding to: position 428 according to SEQ ID NO:32, position 217 according to SEQ ID NO:33, or position 137 according to SEQ ID NO:34; and c) determining whether the extension product of the primer comprises a thymine at a position corresponding to: position 428 according to SEQ ID NO: 32, position 217 according to SEQ ID NO:33, or position 137 according to SEQ ID NO: 34.

In some embodiments, the determining step, detecting step, or sequence analysis comprises: a) contacting the biological sample with: i) a first primer hybridizing to a portion of the nucleotide sequence of the PCSK9 genomic nucleic acid molecule that is proximate to a position corresponding to position 427 according to SEQ ID NO:2, and ii) a second primer hybridizing to a portion of the nucleotide sequence of the LDLR genomic nucleic acid molecule that is proximate to position 2,269 according to SEQ ID NO:4; b) extending the first primer at least through the position of the nucleotide sequence of the PCSK9 genomic nucleic acid molecule corresponding to position 427 according to SEQ ID NO:2; and extending the second primer at least through the position of the nucleotide sequence of the LDLR genomic nucleic acid molecule corresponding to position 2,269 according to SEQ ID NO: 4; and c) determining whether the extension product of the first primer comprises a thymine at the position corresponding to position 427 according to SEQ ID NO:2; and determining whether the extension product of the second primer comprises a thymine at the position corresponding to position 2,269 according to SEQ ID NO:4.

In some embodiments, the assay comprises sequencing the entire nucleic acid molecule. In some embodiments, only a PCSK9 genomic nucleic acid and/or an LDLR genomic nucleic acid molecule is analyzed. In some embodiments, only a PCSK9 mRNA molecule is analyzed. In some embodiments, only a PCSK9 cDNA molecule obtained from a mRNA molecule is analyzed.

In some embodiments, the determining step, detecting step, or sequence analysis comprises: a) amplifying at least a portion of: i) the genomic nucleic acid molecule that encodes the PCSK9 polypeptide and/or the LDLR polypeptide, wherein the amplified portion comprises: a thymine at a position corresponding to position 427 according to SEQ ID NO:2, or the complement thereof; or a thymine at a position corresponding to position 2,269 according to SEQ ID NO:4, or the complement thereof; ii) the PCSK9 mRNA molecule that encodes the PCSK9 polypeptide, wherein the amplified portion comprises a uracil at a position corresponding to: position 428 according to SEQ ID NO:17, or the complement thereof; position 217 according to SEQ ID NO:18, or the complement thereof; or position 137 according to SEQ ID NO: 19, or the complement thereof; or iii) the PCSK9 cDNA molecule that encodes the PCSK9 polypeptide, wherein the amplified portion comprises a thymine at a position corresponding to: position 428 according to SEQ ID NO:32, or the complement thereof; position 217 according to SEQ ID NO:33, or the complement thereof; or position 137 according to SEQ ID NO:34, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: i) a thymine at a position corresponding to position 427 according to SEQ ID NO:2, or the complement thereof; a thymine at a position corresponding to position 2,269 according to SEQ ID NO:4, or the complement thereof; ii) a uracil at a position corresponding to: position 428 according to SEQ ID NO:17, or the complement thereof; position 217 according to SEQ ID NO: 18, or the complement thereof; or position 137 according to SEQ ID NO:19, or the complement thereof; and/or iii) a thymine at a position corresponding to: position 428 according to SEQ ID NO:32, or the complement thereof; position 217 according to SEQ ID NO: 33, or the complement thereof; or position 137 according to SEQ ID NO:34, or the complement thereof; and d) detecting the detectable label.

In some embodiments, the determining step, detecting step, or sequence analysis comprises: a) amplifying at least a portion of the nucleic acid molecule that encodes the PCSK9 and/or the LDLR polypeptide, wherein the amplified portion comprises: a thymine at a position corresponding to position 427 according to SEQ ID NO:2, or the complement thereof; or a thymine at a position corresponding to position 2,269 according to SEQ ID NO: 4, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 427 according to SEQ ID NO:2, or the complement thereof; or a thymine at a position corresponding to position 2,269 according to SEQ ID NO:4, or the complement thereof; and d) detecting the detectable label.

In some embodiments, the determining step, detecting step, or sequence analysis comprises: a) amplifying at least a portion of the nucleic acid molecule that encodes the PCSK9 polypeptide, wherein the amplified portion comprises a uracil at a position corresponding to: position 428 according to SEQ ID NO:17, or the complement thereof; position 217 according to SEQ ID NO:18, or the complement thereof; or position 137 according to SEQ ID NO:19, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a uracil at a position corresponding to: position 428 according to SEQ ID NO:17, or the complement thereof; position 217 according to SEQ ID NO:18, or the complement thereof; or position 137 according to SEQ ID NO:19, or the complement thereof; and d) detecting the detectable label.

In some embodiments, the determining step, detecting step, or sequence analysis comprises: a) amplifying at least a portion of the nucleic acid molecule that encodes the PCSK9 polypeptide, wherein the amplified portion comprises a thymine at a position corresponding to: position 428 according to SEQ ID NO:32, or the complement thereof; position 217 according to SEQ ID NO:33, or the complement thereof; or position 137 according to SEQ ID NO:34, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to: position 428 according to SEQ ID NO:32, or the complement thereof; position 217 according to SEQ ID NO:33, or the complement thereof; or position 137 according to SEQ ID NO:34, or the complement thereof; and d) detecting the detectable label.

In some embodiments, the nucleic acid molecule is mRNA and the determining step further comprises reverse-transcribing the mRNA into a cDNA prior to the amplifying step. In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of: i) the amplified genomic nucleic acid molecule comprising: a thymine at a position corresponding to position 427 according to SEQ ID NO:2, or the complement thereof; a thymine at a position corresponding to position 2,269 according to SEQ ID NO:4, or the complement thereof; ii) the amplified mRNA molecule comprising a uracil at a position corresponding to: position 428 according to SEQ ID NO:17, or the complement thereof; position 217 according to SEQ ID NO:18, or the complement thereof; or position 137 according to SEQ ID NO:19, or the complement thereof; and/or iii) the amplified cDNA molecule comprising a thymine at a position corresponding to: position 428 according to SEQ ID NO:32, or the complement thereof; position 217 according to SEQ ID NO: 33, or the complement thereof; or position 137 according to SEQ ID NO:34, or the complement thereof; and detecting the detectable label.

In some embodiments, the determining step, detecting step, or sequence analysis comprises: contacting the genomic nucleic acid molecule in the biological sample with an alteration-specific probe comprising a detectable label, wherein the alteration-specific probe comprises a nucleotide sequence which hybridizes under stringent conditions to the nucleotide sequence of the amplified PCSK9 nucleic acid molecule and/or LDLR nucleic acid molecule comprising: a thymine at a position corresponding to position 427 according to SEQ ID NO:2, or the complement thereof, or a thymine at a position corresponding to position 2,269 according to SEQ ID NO:4, or the complement thereof, respectively; and detecting the detectable label.

In some embodiments, the determining step, detecting step, or sequence analysis comprises: contacting the mRNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a uracil at a position corresponding to: position 428 according to SEQ ID NO:17, or the complement thereof; position 217 according to SEQ ID NO:18, or the complement thereof; or position 137 according to SEQ ID NO:19, or the complement thereof; and detecting the detectable label.

In some embodiments, the determining step, detecting step, or sequence analysis comprises: contacting the cDNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to: position 428 according to SEQ ID NO:32, or the complement thereof; position 217 according to SEQ ID NO:33, or the complement thereof; or position 137 according to SEQ ID NO:34, or the complement thereof; and detecting the detectable label.

In some embodiments, the determining step, detecting step, or sequence analysis comprises: contacting the genomic nucleic acid molecule in the biological sample with a first alteration-specific probe comprising a first detectable label, wherein the first alteration-specific probe comprises a nucleotide sequence which hybridizes under stringent conditions to the nucleotide sequence of the amplified nucleic acid molecule comprising a thymine at a position corresponding to position 427 according to SEQ ID NO:2, or the complement thereof; and contacting the nucleic acid molecule in the biological sample with a second alteration-specific probe comprising a second detectable label, wherein the second alteration-specific probe comprises a nucleotide sequence which hybridizes under stringent conditions to the nucleotide sequence of the amplified nucleic acid molecule comprising a thymine at a position corresponding to position 2,269 according to SEQ ID NO:4, or the complement thereof; and detecting the first and the second detectable labels.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a PCSK9 variant genomic sequence, variant mRNA sequence, or variant cDNA sequence, and/or an LDLR variant genomic sequence, and not the corresponding PCSK9 reference and/or LDLR reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a PCSK9 variant genomic nucleic acid molecule, variant mRNA molecule, or variant cDNA molecule, and/or an LDLR variant genomic nucleic acid molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a PCSK9 nucleic acid molecule (genomic nucleic acid molecule, mRNA molecule, or cDNA molecule), or complement thereof, and/or an LDLR nucleic acid molecule (genomic nucleic acid molecule), or complement thereof, within a biological sample comprises a nucleotide sequence comprising: i) a thymine at a position corresponding to position 427 according to SEQ ID NO: 2, or a thymine at a position corresponding to position 2,269 according to SEQ ID NO:4 (genomic nucleic acid molecules), ii) a uracil at a position corresponding to: position 428 according to SEQ ID NO:17, position 217 according to SEQ ID NO:18, or position 137 according to SEQ ID NO:19 (mRNA molecules), or iii) a thymine at a position corresponding to: position 428 according to SEQ ID NO:32, position 217 according to SEQ ID NO:33, or position 137 according to SEQ ID NO:34 (cDNA molecules), the biological sample can be subjected to an amplification method using a primer pair that includes a first primer derived from the 5′ flanking sequence adjacent to: i) a thymine at a position corresponding to position 427 according to SEQ ID NO:2, or a thymine at a position corresponding to position 2,269 according to SEQ ID NO:4; ii) a uracil at a position corresponding to: position 428 according to SEQ ID NO:17, position 217 according to SEQ ID NO:18, or position 137 according to SEQ ID NO: 19; or iii) a thymine at a position corresponding to: position 428 according to SEQ ID NO:32, position 217 according to SEQ ID NO:33, or position 137 according to SEQ ID NO:34; and a second primer derived from the 3′ flanking sequence adjacent to: i) a thymine at a position corresponding to position 427 according to SEQ ID NO: 2, or a thymine at a position corresponding to position 2,269 according to SEQ ID NO:4; ii) a uracil at a position corresponding to: position 428 according to SEQ ID NO:17, position 217 according to SEQ ID NO: 18, or position 137 according to SEQ ID NO:19; or iii) a thymine at a position corresponding to: position 428 according to SEQ ID NO:32, position 217 according to SEQ ID NO:33, or position 137 according to SEQ ID NO:34; to produce an amplicon that is indicative of the presence of the SNP at positions encoding: i) a thymine at a position corresponding to position 427 according to SEQ ID NO:2, or a thymine at a position corresponding to position 2,269 according to SEQ ID NO:4; ii) a uracil at a position corresponding to: position 428 according to SEQ ID NO:17, position 217 according to SEQ ID NO: 18, or position 137 according to SEQ ID NO: 19; or iii) a thymine at a position corresponding to: position 428 according to SEQ ID NO:32, position 217 according to SEQ ID NO: 33, or position 137 according to SEQ ID NO:34. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising: i) a thymine at a position corresponding to position 427 according to SEQ ID NO:2, or a thymine at a position corresponding to position 2,269 according to SEQ ID NO: 4; ii) a uracil at a position corresponding to: position 428 according to SEQ ID NO:17, position 217 according to SEQ ID NO:18, or position 137 according to SEQ ID NO:19; or iii) a thymine at a position corresponding to: position 428 according to SEQ ID NO:32, position 217 according to SEQ ID NO:33, or position 137 according to SEQ ID NO:34; and at least 1, 2, 3, 4, 5, 6, 7, 8, 9, 10, or more nucleotides on each side of positions comprising: i) a thymine at a position corresponding to position 427 according to SEQ ID NO:2, or a thymine at a position corresponding to position 2,269 according to SEQ ID NO:4; ii) a uracil at a position corresponding to: position 428 according to SEQ ID NO:17, position 217 according to SEQ ID NO: 18, or position 137 according to SEQ ID NO: 19; or iii) a thymine at a position corresponding to: position 428 according to SEQ ID NO:32, position 217 according to SEQ ID NO: 33, or position 137 according to SEQ ID NO:34.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6X sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a PCSK9 predicted loss-of-function polypeptide comprising performing an assay on a biological sample obtained from a subject to determine whether a PCSK9 polypeptide in the subject contains one or more variations that causes the polypeptide to have a loss-of-function (partial or complete) or predicted loss-of-function (partial or complete). The PCSK9 predicted loss-of-function polypeptide can be any of the PCSK9 variant polypeptides described herein. In some embodiments, the methods detect the presence of the PCSK9Arg46Leu variant polypeptide.

In some embodiments, the methods comprise performing an assay on a sample obtained from a subject to determine whether a PCSK9 polypeptide in the sample comprises a leucine at a position corresponding to position 46 according to SEQ ID NO:42.

In some embodiments, the detecting step comprises sequencing at least a portion of the polypeptide that comprises a position corresponding to position 46 according to SEQ ID NO: 42 or SEQ ID NO:35.

In some embodiments, the detecting step comprises an immunoassay for detecting the presence of a polypeptide that comprises a position corresponding to position 46 according to SEQ ID NO:42 or SEQ ID NO:35.

In some embodiments, when the subject does not have a PCSK9 predicted loss-of-function polypeptide, the subject has an increased risk of developing sepsis, SIRS, septic shock, and/or MODS. In some embodiments, when the subject has a PCSK9 predicted loss-of-function polypeptide, the subject has a decreased risk of developing sepsis, SIRS, septic shock, and/or MODS.

The present disclosure also provides isolated nucleic acid molecules that hybridize to a PCSK9 variant genomic nucleic acid molecule, and/or an LDLR variant genomic nucleic acid molecule, PCSK9 variant mRNA molecules, and/or PCSK9 variant cDNA molecules (such as any of the genomic variant nucleic acid molecules, mRNA variant molecules, and cDNA variant molecules disclosed herein). In some embodiments, the isolated nucleic acid molecules hybridize to a portion of the PCSK9 nucleic acid molecule and/or an LDLR nucleic acid molecule that includes a position corresponding to: i) position 427 according to SEQ ID NO: 2, or position 2,269 according to SEQ ID NO:4; ii) position 428 according to SEQ ID NO: 17, position 217 according to SEQ ID NO:18, or position 137 according to SEQ ID NO:19, or iii) position 428 according to SEQ ID NO:32, position 217 according to SEQ ID NO:33, or position 137 according to SEQ ID NO:34.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to PCSK9 variant nucleic acid molecules (such as genomic nucleic acid molecules, mRNA molecules, and/or cDNA molecules) and/or an LDLR variant nucleic acid molecule (such as a genomic nucleic acid molecule) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to a PCSK9 variant genomic nucleic acid molecule and/or an LDLR variant genomic nucleic acid molecule, PCSK9 variant mRNA molecules, and/or PCSK9 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a PCSK9 polypeptide and/or an LDLR polypeptide, wherein the portion comprises a position corresponding to: i) position 247 according to SEQ ID NO:17, or the complement thereof; or position 2,269 according to SEQ ID NO: 4, or the complement thereof; ii) position 428 according to SEQ ID NO:17, or the complement thereof; position 217 according to SEQ ID NO:18, or the complement thereof; or position 137 according to SEQ ID NO: 19, or the complement thereof; or iii) position 428 according to SEQ ID NO:32, or the complement thereof; position 217 according to SEQ ID NO: 33, or the complement thereof; or position 137 according to SEQ ID NO:34, or the complement thereof. In some embodiments, the alteration-specific probe or alteration-specific primer comprises a nucleotide sequence which is complementary to a portion of a nucleotide sequence comprising positions corresponding to: i) positions 426 to 428 according to SEQ ID NO:2, or the complement thereof; ii) positions 427 to 429 according to SEQ ID NO:17, or the complement thereof; positions 216 to 218 according to SEQ ID NO:18, or the complement thereof; or positions 136 to 138 according to SEQ ID NO:19, or the complement thereof; and/or iii) positions 427 to 429 according to SEQ ID NO:32, or the complement thereof; positions 216 to 218 according to SEQ ID NO:33, or the complement thereof; or positions 136 to 138 according to SEQ ID NO:34, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within the PCSK9 variant genomic nucleic acid molecule and/or the LDLR variant genomic nucleic acid molecule, any of the PCSK9 variant mRNA molecules, and/or any of the PCSK9 variant cDNA molecules disclosed herein. The primers described herein can be used to amplify a PCSK9 variant genomic nucleic acid molecule and/or an LDLR variant genomic nucleic acid molecule, any of the PCSK9 variant mRNA molecules, or any of the PCSK9 variant cDNA molecules described herein, or any fragment thereof.

The present disclosure also provides pairs of primers comprising any of the primers described above. For example, if one of the primers' 3′-ends hybridizes to a guanine at a position corresponding to position 427 according to SEQ ID NO:1 (rather than a thymine) in a particular a PCSK9 genomic nucleic acid molecule, then the presence of the amplified fragment would indicate the presence of the PCSK9 reference genomic nucleic acid molecule. Conversely, if one of the primers' 3′-ends hybridizes to a thymine at a position corresponding to position 427 according to SEQ ID NO:2 (rather than a guanine) in a particular PCSK9 genomic nucleic acid molecule, then the presence of the amplified fragment would indicate the presence of the PCSK9 variant genomic nucleic acid molecule. In some embodiments, the nucleotide of the primer complementary to the thymine at a position corresponding to position 427 according to SEQ ID NO:2 can be at the 3′ end of the primer. In addition, if one of the primers' 3′-ends hybridizes to a guanine at a position corresponding to: position 428 according to SEQ ID NO:17, position 217 according to SEQ ID NO: 18, or position 137 according to SEQ ID NO:19 (rather than a uracil) in a particular PCSK9 mRNA molecule, then the presence of the amplified fragment would indicate the presence of a PCSK9 reference mRNA molecule. Conversely, if one of the primers' 3′-ends hybridizes to a uracil at a position corresponding to: position 428 according to SEQ ID NO:17, position 217 according to SEQ ID NO:18, or position 137 according to SEQ ID NO:19 (rather than a guanine) in a particular PCSK9 mRNA molecule, then the presence of the amplified fragment would indicate the presence of a PCSK9 variant mRNA molecule. In some embodiments, the nucleotide of the primer complementary to the uracil at a position corresponding to: position 428 according to SEQ ID NO:17, position 217 according to SEQ ID NO:18, or position 137 according to SEQ ID NO: 19, can be at the 3′ end of the primer. In addition, if one of the primers' 3′-ends hybridizes to a guanine at a position corresponding to: position 428 according to SEQ ID NO:32, position 217 according to SEQ ID NO:33, or position 137 according to SEQ ID NO:34 (rather than a thymine) in a particular PCSK9 cDNA molecule, then the presence of the amplified fragment would indicate the presence of a PCSK9 reference cDNA molecule. Conversely, if one of the primers' 3′-ends hybridizes to a thymine at a position corresponding to: position 428 according to SEQ ID NO:32, position 217 according to SEQ ID NO:33, or position 137 according to SEQ ID NO:34 (rather than a guanine) in a particular PCSK9 cDNA molecule, then the presence of the amplified fragment would indicate the presence of a PCSK9 variant cDNA molecule. In some embodiments, the nucleotide of the primer complementary to: the thymine at a position corresponding to position 428 according to SEQ ID NO:32, the thymine at a position corresponding to position 217 according to SEQ ID NO:33, or the thymine at a position corresponding to position 137 according to SEQ ID NO:34, can be at the 3′ end of the primer.

In the context of the disclosure “specifically hybridizes” means that the probe or primer (such as, for example, the alteration-specific probe or alteration-specific primer) does not hybridize to a nucleic acid sequence encoding a PCSK9 reference genomic nucleic acid molecule and/or an LDLR reference genomic nucleic acid molecule, a PCSK9 reference mRNA molecule, and/or a PCSK9 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The present disclosure also provides molecular complexes comprising or consisting of any of the PCSK9 nucleic acid molecules (genomic nucleic acid molecules, mRNA molecules, or cDNA molecules), or complement thereof, described herein and any of the alteration-specific primers or alteration-specific probes described herein. The present disclosure also provides molecular complexes comprising or consisting of any of the LDLR nucleic acid molecules (genomic nucleic acid molecules), or complement thereof, described herein and any of the alteration-specific primers or alteration-specific probes described herein. In some embodiments, the PCSK9 nucleic acid molecules (genomic nucleic acid molecule, mRNA molecules, or cDNA molecules), or complement thereof, and/or the LDLR nucleic acid molecule (genomic nucleic acid molecule), or complement thereof, in the molecular complexes are single-stranded. In some embodiments, the PCSK9 nucleic acid molecule and/or the LDLR nucleic acid molecule is any of the genomic nucleic acid molecules described herein. In some embodiments, the PCSK9 nucleic acid molecule is any of the mRNA molecules described herein. In some embodiments, the PCSK9 nucleic acid molecule is any of the cDNA molecules described herein. In some embodiments, the molecular complex comprises or consists of any of the PCSK9 nucleic acid molecules (genomic nucleic acid molecules, mRNA molecules, or cDNA molecules), or complement thereof, described herein and any of the alteration-specific primers described herein. In some embodiments, the molecular complex comprises or consists of any of the LDLR nucleic acid molecules (genomic nucleic acid molecules), or complement thereof, described herein and any of the alteration-specific primers described herein. In some embodiments, the molecular complex comprises or consists of any of the PCSK9 nucleic acid molecules (genomic nucleic acid molecule, mRNA molecules, or cDNA molecules), or complement thereof, described herein and any of the alteration-specific probes described herein. In some embodiments, the molecular complex comprises or consists of any of the LDLR nucleic acid molecules (genomic nucleic acid molecule), or complement thereof, described herein and any of the alteration-specific probes described herein.

In some embodiments, the molecular complex comprises or consists of an alteration-specific primer or an alteration-specific probe hybridized to a genomic nucleic acid molecule comprising a nucleotide sequence encoding a PCSK9 polypeptide, wherein the alteration-specific primer or the alteration-specific probe is hybridized to a thymine at a position corresponding to position 427 according to SEQ ID NO:2, or the complement thereof. In some embodiments, the molecular complex comprises or consists of an alteration-specific primer or an alteration-specific probe hybridized to a genomic nucleic acid molecule comprising a nucleotide sequence encoding an LDLR polypeptide, wherein the alteration-specific primer or the alteration-specific probe is hybridized to a thymine at a position corresponding to position 2,269 according to SEQ ID NO:4, or the complement thereof.

In some embodiments, the molecular complex comprises or consists of an alteration-specific primer or an alteration-specific probe that is hybridized to a CTT codon at positions corresponding to positions 426 to 428 according to SEQ ID NO:2.

In some embodiments, the molecular complex comprises or consists of a genomic nucleic acid molecule that comprises SEQ ID NO:2. In some embodiments, the molecular complex comprises or consists of a genomic nucleic acid molecule that comprises SEQ ID NO: 4.

In some embodiments, the molecular complex comprises or consists of an alteration-specific primer or an alteration-specific probe hybridized to a PCSK9 mRNA molecule comprising a nucleotide sequence encoding a PCSK9 polypeptide, wherein the alteration-specific primer or the alteration-specific probe is hybridized to: a uracil at a position corresponding to position 428 according to SEQ ID NO:17, or the complement thereof; a uracil at a position corresponding to position 217 according to SEQ ID NO:18, or the complement thereof; or a uracil at a position corresponding to position 137 according to SEQ ID NO:19, or the complement thereof.

In some embodiments, the molecular complex comprises or consists of an alteration-specific primer or an alteration-specific probe that is hybridized to a CUU codon at positions corresponding to: positions 427 to 429 according to SEQ ID NO:17, positions 216 to 218 according to SEQ ID NO:18, or positions 136 to 138 according to SEQ ID NO:19.

In some embodiments, the molecular complex comprises or consists of a PCSK9 mRNA molecule that comprises SEQ ID NO:17. In some embodiments, the molecular complex comprises or consists of an mRNA molecule that comprises SEQ ID NO:18. In some embodiments, the molecular complex comprises or consists of an mRNA molecule that comprises SEQ ID NO:19.

In some embodiments, the molecular complex comprises or consists of an alteration-specific primer or an alteration-specific probe hybridized to a PCSK9 cDNA molecule comprising a nucleotide sequence encoding a PCSK9 polypeptide, wherein the alteration-specific primer or the alteration-specific probe is hybridized to a thymine at a position corresponding to: position 428 according to SEQ ID NO:32, or the complement thereof; position 217 according to SEQ ID NO:33, or the complement thereof; or position 137 according to SEQ ID NO:34, or the complement thereof.

In some embodiments, the molecular complex comprises or consists of an alteration-specific primer or an alteration-specific probe that is hybridized to a CTT codon at positions corresponding to: positions 427 to 429 according to SEQ ID NO:32, positions 216 to 218 according to SEQ ID NO:33, or positions 136 to 138 according to SEQ ID NO:34.

In some embodiments, the molecular complex comprises or consists of a PCSK9 cDNA molecule that comprises SEQ ID NO:32. In some embodiments, the molecular complex comprises or consists of a cDNA molecule that comprises SEQ ID NO:33. In some embodiments, the molecular complex comprises or consists of an DNA molecule that comprises SEQ ID NO:34.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of a PCSK9 reference genomic nucleic acid molecule is set forth in SEQ ID NO:1. Referring to SEQ ID NO:1, position 427 is a guanine.

A variant genomic nucleic acid molecule of PCSK9 exists, wherein the guanine at position 427 is replaced with a thymine. The nucleotide sequence of this PCSK9 variant genomic nucleic acid molecule is set forth in SEQ ID NO:2.

The nucleotide sequence of an LDLR reference genomic nucleic acid molecule is set forth in SEQ ID NO:3. Referring to SEQ ID NO:3, position 2,269 is a guanine.

A variant genomic nucleic acid molecule of LDLR exists, wherein the guanine at position 2,269 is replaced with a thymine. The nucleotide sequence of this LDLR variant genomic nucleic acid molecule is set forth in SEQ ID NO:4.

The nucleotide sequence of a first PCSK9 reference mRNA molecule is set forth in SEQ ID NO:5. Referring to SEQ ID NO:5, position 428 is a guanine. The nucleotide sequence of a second PCSK9 reference mRNA molecule is set forth in SEQ ID NO:6. Referring to SEQ ID NO: 6, position 217 is a guanine. The nucleotide sequence of a third PCSK9 reference mRNA molecule is set forth in SEQ ID NO:7. Referring to SEQ ID NO:7, position 137 is a guanine.

A first PCSK9 variant mRNA molecule exists, wherein the guanine at position 428 (referring to SEQ ID NO:5) is replaced with a uracil. The nucleotide sequence of this PCSK9 variant mRNA molecule is set forth in SEQ ID NO:17. The first PCSK9 variant mRNA molecule comprises a CUU codon at positions 427 to 429 according to SEQ ID NO:17.

A second PCSK9 variant mRNA molecule exists, wherein the guanine at position 217 (referring to SEQ ID NO:6) is replaced with a uracil. The nucleotide sequence of this PCSK9 variant mRNA molecule is set forth in SEQ ID NO:18. The second PCSK9 variant mRNA molecule comprises a CUU codon at positions 216 to 218 according to SEQ ID NO:18.

A third PCSK9 variant mRNA molecule exists, wherein the guanine at position 137 (referring to SEQ ID NO:7) is replaced with a uracil. The nucleotide sequence of this PCSK9 variant mRNA molecule is set forth in SEQ ID NO:19. The third PCSK9 variant mRNA molecule comprises a CUU codon at positions 136 to 138 according to SEQ ID NO:19.

The nucleotide sequence of a first PCSK9 reference cDNA molecule is set forth in SEQ ID NO:20. Referring to SEQ ID NO:20, position 428 is a guanine. The nucleotide sequence of a second PCSK9 reference cDNA molecule is set forth in SEQ ID NO:21. Referring to SEQ ID NO:21, position 217 is a guanine. The nucleotide sequence of a third PCSK9 reference cDNA molecule is set forth in SEQ ID NO:22. Referring to SEQ ID NO:22, position 137 is a guanine.

A first PCSK9 variant cDNA molecule exists, wherein the guanine at position 428 (referring to SEQ ID NO:20) is replaced with a thymine. The nucleotide sequence of this PCSK9 variant cDNA molecule is set forth in SEQ ID NO:32. The first PCSK9 variant cDNA molecule comprises a CTT codon at positions 427 to 429 according to SEQ ID NO:32.

A second PCSK9 variant cDNA molecule exists, wherein the guanine at position 217 (referring to SEQ ID NO:21) is replaced with a thymine. The nucleotide sequence of this PCSK9 variant cDNA molecule is set forth in SEQ ID NO:33. The second PCSK9 variant cDNA molecule comprises a CTT codon at positions 216 to 218 according to SEQ ID NO:33.

A third PCSK9 variant cDNA molecule exists, wherein the guanine at position 137 (referring to SEQ ID NO:22) is replaced with a thymine. The nucleotide sequence of this PCSK9 variant cDNA molecule is set forth in SEQ ID NO:34. The third PCSK9 variant cDNA molecule comprises a CTT codon at positions 136 to 138 according to SEQ ID NO:34.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1 (PCSK9 genomic), SEQ ID NO:5, SEQ ID NO:6, or SEQ ID NO:7 (PCSK9 mRNA), or SEQ ID NO:20, SEQ ID NO:21, or SEQ ID NO:22 (PCSK9 cDNA)). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding a PCSK9 polypeptide, wherein the nucleotide sequence comprises a thymine at a position corresponding to position 427 according to SEQ ID NO:2 means that if the nucleotide sequence of the PCSK9 genomic nucleic acid molecule is aligned to the sequence of SEQ ID NO: 2, the PCSK9 sequence has a thymine residue at the position that corresponds to position 427 of SEQ ID NO:2. The same applies for mRNA molecules comprising a nucleotide sequence encoding a PCSK9 polypeptide, wherein the nucleotide sequence comprises a uracil at a position corresponding to position 428 according to SEQ ID NO:17, and cDNA molecules comprising a nucleotide sequence encoding a PCSK9 polypeptide, wherein the nucleotide sequence comprises a thymine at a position corresponding to position 428 according to SEQ ID NO:32. In other words, these phrases refer to a nucleic acid molecule encoding a PCSK9 polypeptide, wherein the genomic nucleic acid molecule has a nucleotide sequence that comprises a thymine residue that is homologous to the thymine residue at position 427 of SEQ ID NO:2 (or wherein the mRNA molecule has a nucleotide sequence that comprises a uracil residue that is homologous to the uracil residue at position 428 of SEQ ID NO: 17, or wherein the cDNA molecule has a nucleotide sequence that comprises a thymine residue that is homologous to the thymine residue at position 428 of SEQ ID NO:32).

As described herein, a position within a PCSK9 genomic nucleic acid molecule and/or an LDLR genomic nucleic acid molecule that corresponds to position 427 according to SEQ ID NO:2 or position 2,269 according to SEQ ID NO:4, respectively, for example, can be identified by performing a sequence alignment between the nucleotide sequence of a particular a PCSK9 nucleic acid molecule and/or an LDLR nucleic acid molecule and the nucleotide sequence of SEQ ID NO:2 or SEQ ID NO:4, respectively. A variety of computational algorithms exist that can be used for performing a sequence alignment to identify a nucleotide position that corresponds to, for example, position 427 in SEQ ID NO:2, or position 2,269 in SEQ ID NO:4. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of a PCSK9 reference polypeptide is set forth in SEQ ID NO: 35. Referring to SEQ ID NO:35, the PCSK9 reference polypeptide is 692 amino acids in length. Referring to SEQ ID NO:35, position 46 is an arginine.

A PCSK9 variant polypeptide exists (Arg46Leu or R46L), the amino acid sequence of which is set forth in SEQ ID NO:42. Referring to SEQ ID NO:42, the PCSK9 variant polypeptide is also 692 amino acids in length. Referring to SEQ ID NO:42, position 46 is a leucine.

The amino acid sequence of a first LDLR reference polypeptide is set forth in SEQ ID NO: 36. Referring to SEQ ID NO:36, the LDLR reference polypeptide is 860 amino acids in length. The amino acid sequence of a second LDLR reference polypeptide is set forth in SEQ ID NO: 37. Referring to SEQ ID NO:37, the LDLR reference polypeptide is 682 amino acids in length. The amino acid sequence of a third LDLR reference polypeptide is set forth in SEQ ID NO: 38. Referring to SEQ ID NO:38, the LDLR reference polypeptide is 692 amino acids in length. The amino acid sequence of a fourth LDLR reference polypeptide is set forth in SEQ ID NO: 39. Referring to SEQ ID NO:39, the LDLR reference polypeptide is 819 amino acids in length. The amino acid sequence of a fifth LDLR reference polypeptide is set forth in SEQ ID NO: 40. Referring to SEQ ID NO:40, the LDLR reference polypeptide is 858 amino acids in length. The amino acid sequence of a sixth LDLR reference polypeptide is set forth in SEQ ID NO: 41. Referring to SEQ ID NO:41, the LDLR reference polypeptide is 739 amino acids in length.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit sepsis, SIRS, septic shock, and/or MODS for use in the treatment of sepsis, SIRS, septic shock, and/or MODS (or for use in the preparation of a medicament for treating sepsis, SIRS, septic shock, and/or MODS) in a subject, wherein the subject has any of the PCSK9 genomic nucleic acid molecules, mRNA molecules, and/or cDNA molecules described herein and/or has any of the LDLR genomic nucleic acid molecules, mRNA molecules, and/or cDNA molecules described herein. The therapeutic agents that treat or inhibit sepsis, SIRS, septic shock, and/or MODS can be any of the therapeutic agents that treat or inhibit sepsis, SIRS, septic shock, and/or MODS described herein. In some embodiments, the subject comprises: i) a genomic nucleic acid molecule having a nucleotide sequence encoding a PCSK9 polypeptide, wherein the nucleotide sequence comprises a thymine at a position corresponding to position 427 according to SEQ ID NO:2, or the complement thereof; and/or a genomic nucleic acid molecule having a nucleotide sequence encoding an LDLR polypeptide, wherein the nucleotide sequence comprises a thymine at a position corresponding to position 2,269 according to SEQ ID NO:4, or the complement thereof; ii) an mRNA molecule having a nucleotide sequence encoding a PCSK9 polypeptide, wherein the nucleotide sequence comprises a uracil at a position corresponding to: position 428 according to SEQ ID NO:17, or the complement thereof; position 217 according to SEQ ID NO: 18, or the complement thereof; or position 137 according to SEQ ID NO:19, or the complement thereof; or iii) a cDNA molecule having a nucleotide sequence encoding a PCSK9 polypeptide, wherein the nucleotide sequence comprises a thymine at a position corresponding to: position 428 according to SEQ ID NO:32, or the complement thereof; position 217 according to SEQ ID NO:33, or the complement thereof; or position 137 according to SEQ ID NO:34, or the complement thereof.

The present disclosure also provides a PCSK9 inhibitor and/or an LDLR agonist for use in the treatment of sepsis, systemic inflammatory response syndrome (SIRS), septic shock, and/or multiple organ dysfunction syndrome (MODS), in a subject. In some embodiments, the subject is reference for a PCSK9 and/or an LDLR genomic nucleic acid molecule, a PCSK9 and/or an LDLR mRNA molecule, or a PCSK9 and/or an LDLR cDNA molecule. In some embodiments, the subject is heterozygous for: i) a genomic nucleic acid molecule having a nucleotide sequence encoding a PCSK9 polypeptide, wherein the nucleotide sequence comprises a thymine at a position corresponding to position 427 according to SEQ ID NO:2; and/or ii) a genomic nucleic acid molecule having a nucleotide sequence encoding an LDLR polypeptide, wherein the nucleotide sequence comprises a thymine at a position corresponding to position 2,269 according to SEQ ID NO:4; an mRNA molecule having a nucleotide sequence encoding a PCSK9 polypeptide, wherein the nucleotide sequence comprises a uracil at a position corresponding to: position 428 according to SEQ ID NO:17, or the complement thereof; position 217 according to SEQ ID NO: 18, or the complement thereof; or position 137 according to SEQ ID NO:19, or the complement thereof; or a cDNA molecule having a nucleotide sequence encoding a PCSK9 polypeptide, wherein the nucleotide sequence comprises a thymine at a position corresponding to: position 428 according to SEQ ID NO:32, or the complement thereof; position 217 according to SEQ ID NO:33, or the complement thereof; or position 137 according to SEQ ID NO:34, or the complement thereof.

In some embodiments, the PCSK9 inhibitor is an antisense nucleic acid molecule, an siRNA, or an shRNA that hybridizes to a PCSK9 mRNA. In some embodiments, the PCSK9 inhibitor comprises a Cas protein and gRNA that hybridizes to a gRNA recognition sequence within a PCSK9 genomic nucleic acid molecule. In some embodiments, the Cas protein is Cas9 or Cpf1. In some embodiments, the gRNA recognition sequence includes or is proximate to position 427 according to SEQ ID NO:1. In some embodiments, the gRNA recognition sequence is located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 427 according to SEQ ID NO:1. In some embodiments, a PAM sequence is about 2 to about 6 nucleotides downstream of the gRNA recognition sequence. In some embodiments, the gRNA comprises from about 17 to about 23 nucleotides. In some embodiments, the gRNA recognition sequence comprises a nucleotide sequence according to any one of SEQ ID NOS: 43-68.

In some embodiments, the LDLR agonist is an antisense nucleic acid molecule, an siRNA, or an shRNA that hybridizes to an LDLR mRNA molecule. In some embodiments, the LDLR agonist comprises a Cas protein and gRNA that hybridizes to a gRNA recognition sequence within an LDLR genomic nucleic acid molecule. In some embodiments, the Cas protein is Cas9 or Cpf1. In some embodiments, the gRNA recognition sequence includes or is proximate to position 2,269 according to SEQ ID NO:3. In some embodiments, the gRNA recognition sequence is located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 2,269 according to SEQ ID NO:3. In some embodiments, a PAM sequence is about 2 to about 6 nucleotides downstream of the gRNA recognition sequence. In some embodiments, the gRNA comprises from about 17 to about 23 nucleotides. In some embodiments, the gRNA recognition sequence comprises a nucleotide sequence according to any one of SEQ ID NOS: 69-89.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Human Genetics for PCSK9 and Sepsis Mortality

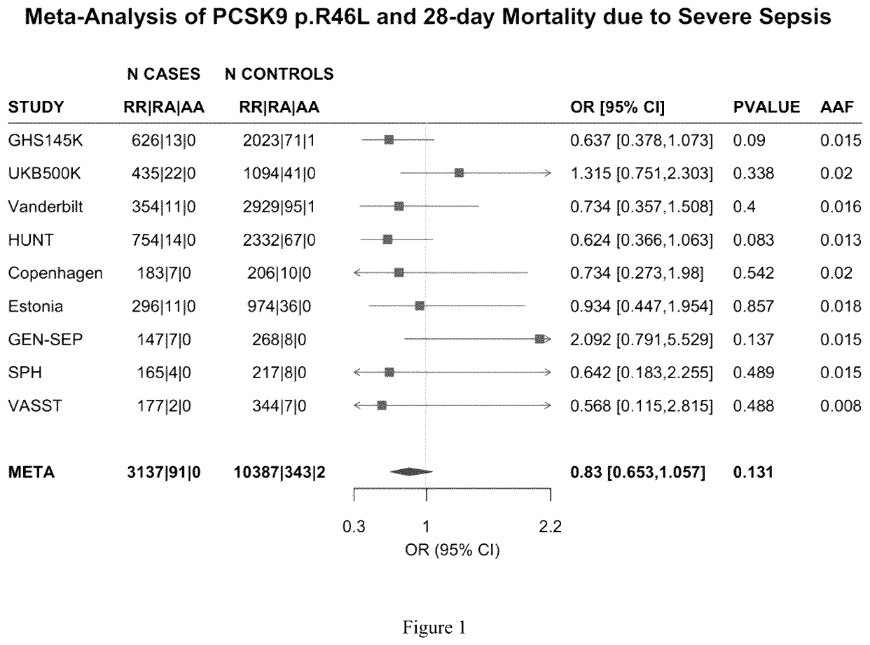

To assess whether loss of function in PCSK9 and/or LDLR associates with improved sepsis survival, the association between loss-of-function in PCSK9 and 28-day mortality due to severe sepsis (as defined by ICD10 codes) was examined using particular genetic data from GHS and UKB cohorts. In addition, summary data was collected and a meta-analysis of the 28-day mortality due to severe sepsis (that was either clinically defined or defined by ICD10 codes for suspected/confirmed infection and acute organ failure) was performed across 9 cohorts, including a total of 3,228 severe sepsis cases who died and 10,732 sepsis cases who survived. The cohorts with summary data that were included in the meta-analysis included GHS, Gen-Sep, Vanderbilt, SPH, VASST, UKB, NTNU, Estonia, and Copenhagen (see, FIG. 1).

The meta-analysis focused on PCSK9 p.Arg46Leu (rs11591147, AAF 0.012), a well-characterized missense variant that acts as a loss-of-function in terms of having a large effect on LDL lowering (in UKB: ref carriers: 138.23 mg/dL, het carriers: 126.8 mg/dL, hom carriers: 103.8 mg/dL (Cohen, N. Eng. J. Med., 2005; p-value=4.9E-324 in UKB), and significantly reduced odds of coronary artery disease (UKB: OR=0.79, p=6.4E-14). Further, in GHS, the LDLR intronic variant (rs6511720, AAF 0.12) with significant effects on LDL lowering (−5.9 mg/dl per allele, p=3.9E-324) and significantly reduced odds of CAD (OR=0.90, p=6.2E-13) was also tested. The results of the meta-analysis for association between PCSK9 p.Arg46Leu and 28-day mortality due to severe sepsis showed an OR=0.83 (0.65, 1.06) and p=0.13, consistent with PCSK9 p.Arg46Leu associating with reduced odds of death due to severe sepsis.

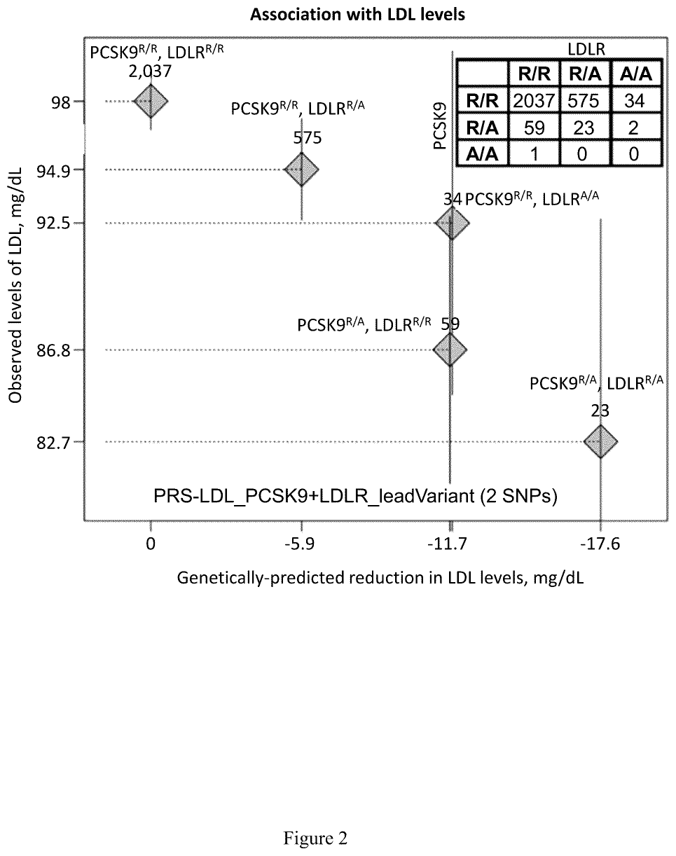

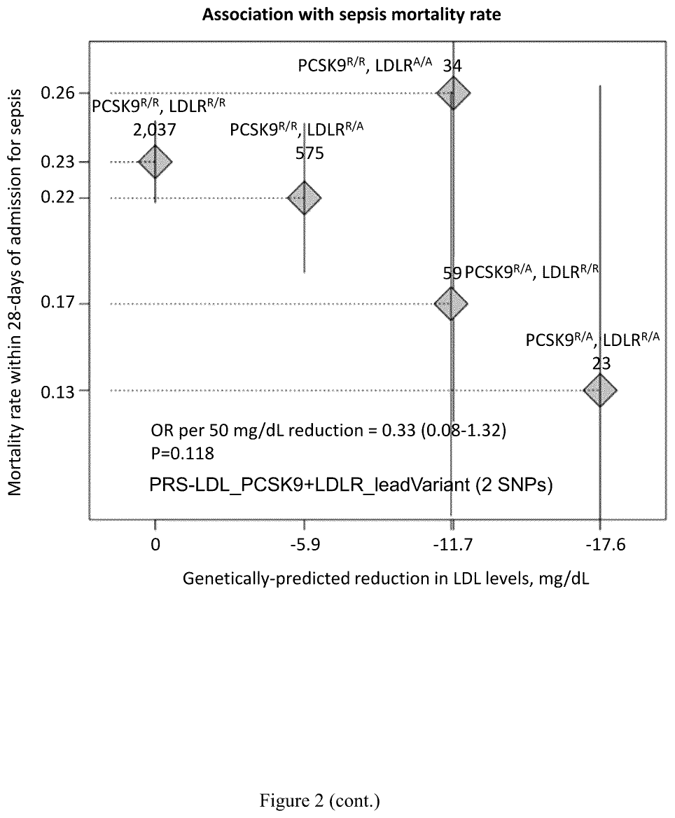

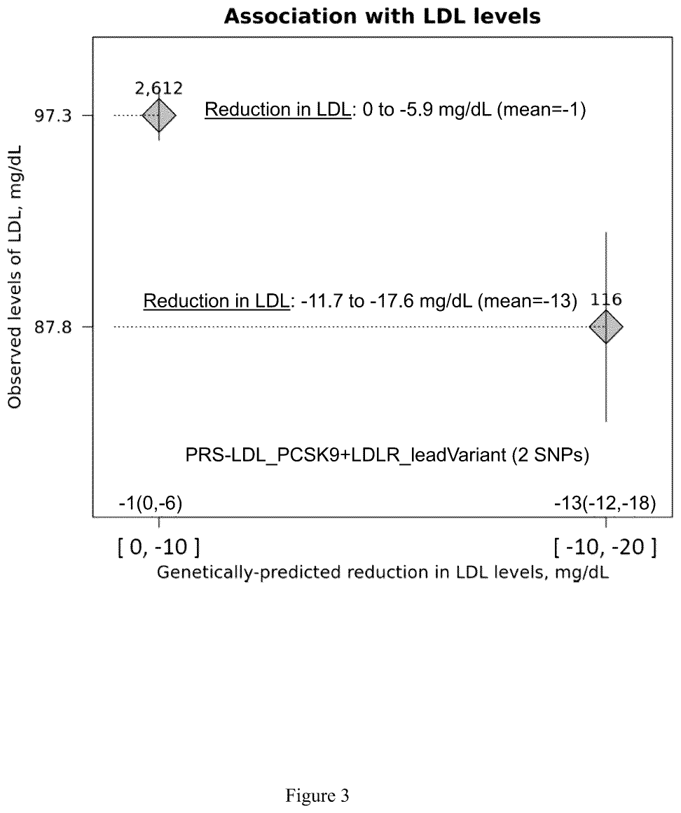

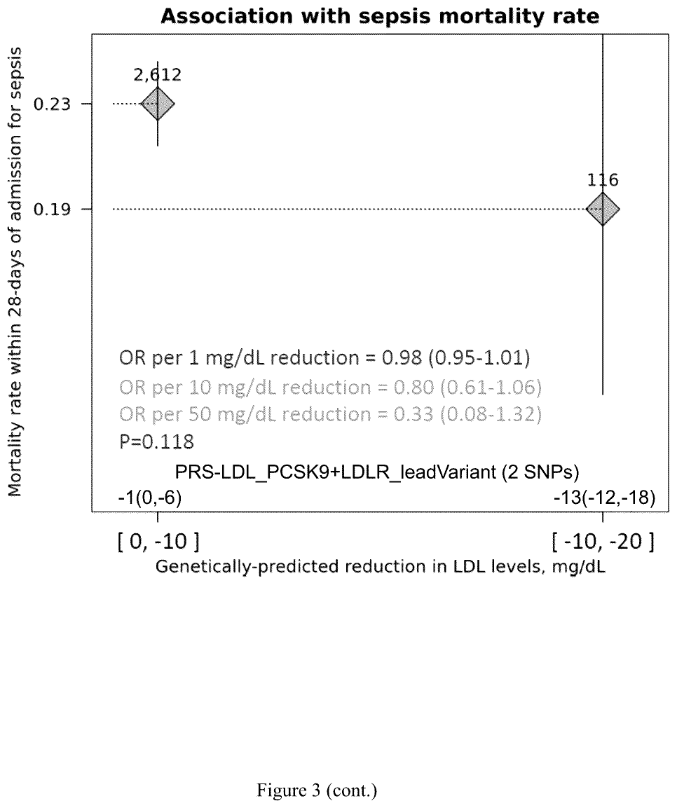

Further, the combined effects of the loss of PCSK9 and LDLR on 28-day sepsis mortality was analyzed. From this analysis, it was found that the combined effects of sepsis mortality on carriers of both PCSK9 p.Arg46Leu and LDLR rs6511720 were OR=0.33 (0.08, 1.32) and p=0.12 (see, FIG. 2 and FIG. 3), consistent with reduced odds of death due to sepsis. Similar combined effects in replication analysis was observed with Estonia and HUNT. In sum, an association between PCSK9 p.R46L and reduced mortality due to severe sepsis was observed.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having sepsis, systemic inflammatory response syndrome (SIRS), septic shock, or multiple organ dysfunction syndrome (MODS), the method comprising administering a Proprotein Convertase Subtilisin/Kexin Type 9 (PCSK9) inhibitor to the subject, wherein the subject is not homozygous for a PCSK9 variant nucleic acid molecule encoding a PCSK9 predicted loss-of-function polypeptide.

2. The method according to claim 1, wherein the sepsis is severe sepsis.

3-5. (canceled)

6. The method according to claim 1, wherein the PCSK9 inhibitor comprises an antisense nucleic acid molecule, a small interfering RNA (siRNA), or a short hairpin RNA (shRNA) that hybridizes to PCSK9 mRNA.

7-17. (canceled)

18. The method according to claim 1, wherein the PCSK9 variant nucleic acid molecule is a nucleic acid molecule encoding Arg46Leu polypeptide.

19. The method according to claim 18, wherein the PCSK9 variant nucleic acid molecule is:
a genomic nucleic acid molecule having a nucleotide sequence comprising a thymine at a position corresponding to position 427 according to SEQ ID NO:2;
an mRNA molecule having a nucleotide sequence comprising a uracil at a position corresponding to: position 428 according to SEQ ID NO: 17, position 217 according to SEQ ID NO: 18, or position 137 according to SEQ ID NO:19; or
a cDNA molecule produced from an mRNA molecule, wherein the cDNA molecule has a nucleotide sequence comprising a thymine at a position corresponding to: position 428 according to SEQ ID NO:32, position 217 according to SEQ ID NO:33, or position 137 according to SEQ ID NO:34.

20-69. (canceled)

70. A method of identifying a subject having a risk of developing sepsis, systemic inflammatory response syndrome (SIRS), septic shock, or multiple organ dysfunction syndrome (MODS), the method comprising:
determining or having determined the presence or absence of a PCSK9 variant nucleic acid molecule encoding a (PCSK9) predicted loss-of-function polypeptide in a biological sample obtained from the subject;
wherein when the subject does not have the PCSK9 variant nucleic acid molecule administering a PCSK9 inhibitor to the subject.

71-116. (canceled)

117. The method according to claim 70, wherein the PCSK9 inhibitor comprises an antisense nucleic acid molecule, a small interfering RNA (siRNA), or a short hairpin RNA (shRNA) that hybridizes to a PCSK9 mRNA.

118-130. (canceled)

131. The method according to claim 70, wherein the PCSK9 inhibitor comprises alirocumab or evolocumab, or a combination thereof.

132. The method according to claim 70, wherein the PCSK9 inhibitor comprises alirocumab.

133. The method according to claim 70, wherein the PCSK9 inhibitor comprises the monoclonal antibody SAR236553/REGN727 or AMG145, or a combination thereof.

134. The method according to claim 1, wherein the PCSK9 inhibitor comprises alirocumab or evolocumab, or a combination thereof.

135. The method according to claim 1, wherein the PCSK9 inhibitor comprises alirocumab.

136. The method according to claim 1, wherein the PCSK9 inhibitor comprises the monoclonal antibody SAR236553/REGN727 or AMG145, or a combination thereof.


SEQUENCE LISTING (89 sequences)

SEQ ID NO: 1 - DNA, 25305 bp, Homo sapiens
  source 1..25305: mol_type = genomic DNA
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggtgcgggtgtagggatgggaggccggggcgaacccgcagccgggacggtgcggtgctgtttcctctcgggcctcagtttccccccatgtaagagaggaagtggagtgcaggtcgccgagggctcttcgcttggcacgatcttggggactgcaggcaaggcggcgggggaggacgggtagtggggagcacggtggagagcggggacggccggctctttggggacttgctggggcgtgcggctgcgctattcagtgggaaggttcgcggggttgggagacccggaggccgaggaagggcgagcagagcactgccaggatatcctgcccagatttcccagtttctgcctcgccgcggcacaggtgggtgaaggagtgaatgcctggaacgtactgggaactgcaccaggcacagagaaagcgggcttgccattatagtgggttccgatttggtttggaaaacatgggcagcggagggtggagggcctggagagaaggccctacccgagacaggggcggggtgggaaggacggcagatgctgggagcacgaggcaatttctttatgacacagaactcatgctctagtattccatctgtttcagccgaagaaaagaaccagctgaaggggcaggggagaaggggcggaggtattctcgaggcccattggcgtcctttaggactcaggcagggaagggcccttggtgctctggagccggaggtggtgcgcctggtactgggaccccggagctgagcccggcgcctcagcccacctggctgtctgccgaccgtgtgcggggcgagtttgctcaacaactctgccagcttctggccctcaggctgtgggaagcttcttcccggggcgagaccactagctttttctaagtattaccagcccaggacttggctgaggttctgtgtcccccagcttggagtcagatgtggggttgaatcttggcttcctctcactagctgtggtgcttgacaagtcacttatccttgagcctccattgcctaatctttaaaagggaggtgacaatcgtccctacggctcagtggcagcagatggggagatgaagggaaagttctgttgaccatgagtgaacttacaatgcaagccccggggggatcacttgcagttttgtccctgtctgcagtgtgacctgttggtgacattgtctttgctccaaaccacagctcctggggcagaggggaaaattctgccactcacagctgcctgcccacgcttctgtctgagtgtgctgggtggcaggatggcaagtccttactcagctcagtatagccctcttccttgttccctgagcctttgactttctcgagggatgttgtggggttgtggccaggataagaaagggcatttcaagttaccactgctccaaaacaactgttctggaaatagtgagtaccccatcctgagaggtgagtaagcagaggctgtatgaccacctgaaccaagcccttgaggatgtttcttctctggtggaagtttggaacaggagcctcctcaagttcatttattcattcattcaatggttattttgtgggaatcgaatttagaatgaaaatattttttggcaagcagaaaataatttttagaccaatccttttcttttagtcatgagaaactgaggcccagagagaggaggtcaccccaggtgcattagaactgggtttccagaactgacactccactgcacagagtactctcccaattcattcaatttttatttagcggaaggcattttcagatgggtctttgaagcattagtaggagttcagcgatgatggtgtcatgagaattttattctaggattaggaggtaccatgaacaaagatacagagctgggaaaaccagaggtggaagataaggagcacatgtccacagttctttttcttttttttttgagatggagtttcgctcttgttgcccaggctggagtgcaatggtgcagtctcagctcactgcaacatctgtctcccgggttcaagtggttctcctgcctcagcctcccaagaagctgggattacaggtacctgccaccacgcccggctaatttttgtatttttagtagagaaggggtttcaccacgttggccaggctagtcgcaaactcctgacctcctcagtggatccgaggaggtgatcctcccgcctcagcctcccaaagtgctcgaattacaggtgtgagccaccacgcctggcctccacagttctttatccaccgtctgaaatgtaaaatgttacgaaaaccaaaagttttttttgtgatttatttgatggtagcacctgacgtgaactgacatgagattatttttaatttagttgtgtgaatatgcatattcatatattttgctgcatagattacagtatgcagctccagattcttccaagcagactctgattgcccattactgcctttctaaaatccaaacaagttctgaggttcaaaaccgttttggccctaaggctttgggtaaagggggtggactctgttctactctgactggagtccaagatgcatatatacagagatatgggtgatggggctgcaaggtaggttgaggtaggggccaaggaggagcatggagtttggacttgattcatgaggctgtggggagccagtgaaggttcttaagcaggtatgtctgcctgagagcagttggagcagacaagagctaaaaaccaaacaaatcaccatagatagtggctgctataatttgtttgtcccctccaaatctcatgtggaaatttggtcctcagtgttggaagtggggcctaatgggaggtgtttgggtcatgggggaggaacccctgtgaaaggcttggtgccgtccttgtgataatgagtaagttctcccgctatgatttcccttgaaggctgattattaaaaagagcttggcacctccctctcttctctcttgcttcttctcttgccatgtgattgatctctgcacatgtaggctccccttcaccttctgccatcagtgaaagcagcttaaggccctcaccagaagcagatgctggtgccatgcttcctggagagcttgcagaatcatgagctgaataaatcccttttccttgtaaattactcaccttcaggtattcctttatatagcaacacaaaaggactaagacagtggccttgacttttctctctctttaagaagtgttgcctttgctcacttagtcatcccttctgcctgcatttgtagagcatctggatgggagatttatataaccgtcactcttgactttcccagcaggcctatgtcataggtactgtggtctctacaatacagcagaggtatctgaggctccgagaggttgagtgacttgctcatggctgcacaaccagtaaatattggagctggaattcaggtccacggtttcctggctccaaagcccatgattttttccctcaatttattctgactggggcatgggggagggggtggcctttgggcagggccaccaggagcgaccaggcccgtagagagctgggtgcaggtacagaggaaaacctgttgtcgagtgtggcccgtagttcccatttttgcctgaatggcacatttgaaagtgttatataaccatgtgaataataatagttggcctatatgagttctttaatttgctttttggtccgcatttggtaacttctttatcatctactatactctgttgtgtctcttttgttgtaatttgtaagtaggggtgagataaagtacacctagggtttgctgggtttcttccatgtcatcatgttcctccttgcatggggccaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggtgagccaccctttttgggaatggcacttcctgatagggctgggccactgcatatacactggggactgtgcttagtaggcccattgctgaaaatcagaaggggacagcaagtatgtattgagcacttatcgggtaccaagcacagtaactactggctttctgtatagaattccctttaagcctggccatgccccagtggtacgtctatcttcatttgaaagacgaggagactgaagttcagaggggaccacacagacagctaggggtagagcctggatcaaacccattggtctgcctgccagccattcttgtgccaatgcatctgctgcctacggaaacctgtagggacaaggccctgggatgttcagtggagcctgagtcattttataaaaaagcatgactctagggtccaaaattcctttgaagctgttgctatccagagtgaagtcccttctttaggacagggtggccctcctccctcctggatgtcacatcttcggtggaggggcagaaaggggactgggtattctcctcaccctggccctagtgcttcaaatcttaaaaaaacgtttttatttgtgcttctgcaccaccttctagcccacctcgtttcctggcctctaacttgatgagagcgtgtgtcattttcacactgattctccacatggcaggcggtgcttcttagcctcctgcagacagtgaggccccacggtcttgtccaaggtcacacagcgtgtaatgggcagggtcagagtctggagtctggacctgggtctcctagctgcactgcactgctgccccatgggttaatcagctcagcataccgtggctgaacagctacctcataccaaggcctgtggcgccatgacagggattgacagggtccctgccttggaaacccgtagtctaagtagaggagactgacaagtcaatgccttccatcagtctgctcaacacacgtttaccaagtgcctactgtgtgctgcagaggcgaagatgacacagctcaggcctttcccttgagcttacagttcaggaggagagactgaccagtgactgccagtacagttgactatgggacaatgtgctcagccttggggagagacgaagaaggtacccgtatagcaccagatgacaggcacgagccccacaggccagggcagctgctcagaggagagtaggccaagcagaaggcaaacagaaggctgcaggcatttgccatcgagagctggacttcaaactgggcatcataccagcctgggttcgagtcctgcccagccccttattggctgtctaaccctgagcaaatcccttcacctctctgagcctcattcctctatctgtaaaccagttataataattggaacattcatttaaggactaaatgaggtcgtgaagcattcagcagatgctaggtacggaaactcgctgaagtgggggcaggttaagaagcctctggggatacgaaggcatccagggactagttgtggcaggaggctgttaccacttaggtctgaagggtaaggagagggaatagctttccctctgcccagttggagccggtggcatggaggagaggctgcctgtggggaatcacccgagggttcaccgctgccatgcgcagggagtcaggaggtagggagggagtggggcagatgcacaccatttttttttttttttgagactctgttgcccagactggagtgcagtggtgccatatctgcacctctgcctcccgggttcaagctcactgcaacctctgcctcccgggttcaagcgattctcctgcctcagcctcccgagtagctgggactacaggtgtgtgccaccatgcctggctaatttttgtatttttaatagagatggggtttcaccatgttggccaggctggtctcgaactctcgacctcaggtgatcccccacctcggcctcccaaagtgctgggattacaggcgtgagtcaccgctcccagctgctgatgcactcttgtccttctaactcctgctagtgcctcccattggctgagcccaactggaagctttgcaagggagctggtgctgcagtttgcactgagcaggctggagaaggctggagaatagactaggggacaaaccgaattgccagtgctgttatgtcatgatttaggcatggagtccagggcctgagcttcactccatgtccatcctgcccagagccttggcacagcctggctcccagacaagatgtcaagttcagaatccttcctaaaaggaatcctctatgccagaccgtgttgcagggatatgggagtgctgggctcccagcctgatcaaggagcgagaaaactcaggctcctagtctgtcctccggggcactagcagggacaaggtgggaggctgctgggctgggatgtggggacaggtttgatcaggtaaggccaggctgtggctgtgtttgctgctgtccaaatggcttaagcagagtcccccggcctctctggcttctgcaggccttgaagttgccccatgtcgactacatcgaggaggactcctctgtctttgcccagagcatcccgtggaacctggagcggattacccctccacggtaccgggcggatgaataccagccccccggtaagacccccatctgtgccctgccccaccccatctgagctgaatccatttgctctgccctggcctggcctccctgctggtggtttccacttctcggggggctttgggactcagcacctccactgacccctttttttctgtcccatccccatcccctgcagcccccactgcctgccttcctgttgccccacaaatgcaaaagtcttgccttaaatgatcctcttttccttcttttctcttgttttccttttctcaccatttggaatggcccagcaggctgcacttaccttggaaggagggttcatctgatggtgactctacctagggcccccaggcctctataactcccagtgccctgcagactggaccagatcctttaatgggatagacacaaccctgtctgggatgcctctgcctaccttcctgttttgctgctccacctgcctccagctccgtttggcttcctggggctccctgcctgggccactttgtgtcttccctctaggcctttctttccactgttccctctgcctggtgtggcctggctatggaagggagggaggaggagcggccatggaaaacggtctgcattctagcagggacttgcaggtggcaattcagtcggggaagactctagatgcacctggcctgaggagagaatgaagggttctagttggactgtgttaagtttgaggtgcccatggtgtgaggtctggagctcagcgcagagatgatgcaatgtggtgggtccatgcaacatggtgccaggacgcagagcttggggtgaactcagctttcaccccttaccggttctcgtgggatcttgggaagccactttcttctatgagctttgtcgttcttgtctgtaaaatgggcacataaccctgtccctgtccttctcacaggttgctgtgagactccaatgagttgaaggatgtgcagatgcttttggaagtgaaaagttggggggctactgtgtgactttgcatacacccaaactgtgtgaccttgcatatgtctgagttgctgccattgcaacagatcagagctggtgggctgggtgtggagaaagggtttgtgtgggggacatcctctggcaagggtggcagcagcagaagtgaggggcctggtcggtcatgtgtgctgacccggcctgggcagcctgtggccagggagaggacagctcctctgtaggaagagcctgttcctttccaaccaggtgagacctcttcagtggagccctggagccccctgtactccacatcagtgcctcagggacctcccggagcaggctaatatcagagaccaagagggacactggcagaggatcacagagaccccagtccaggcagggactgagaagatcttgccccctaagttagtttcctagcactgctgtgacaaattaccaccccctcggttggaacaagttgattctctgcagtcctggaggccagaagcctgaatcagtgtcggcaggaccactttctcccggggggctccagggagaagcttctcttgcctcttccgtgtcccaacagcggcagcacaccaatcccagcctctgtcttcacacagccttctctgtgtctctctcctcttcattgtctcataaggacacttgtcattggatttagggcccactggatcctccaggatgatctcatgtggggaaccttaaccacatctgcaaggaccctttttccaaataaggtcacagccacaggttgtgggggttaggatgtgagtgtatctctttggcagccactgttccctcctctcccttgggccagaagcagacgtggggccctttcttccccataggatgcccatggattgccccccttcccgcttcccccgagtgtctgtgggaggtggcaggaatggcaggcaggggtgtggaaccccttctggagtcatatcaagggcttggctggaggaagtcctcctggagctgttgggctggcatggggcaggctggctgggcccagcagcagcttcttcattcatggggaggccacaagcatgggccctagagctggctgccgccctcaaacccagaccctgcactcttaactgtgtgaccttgcatacgtcactcaccctctctgatcttcaggttcctctgcaaaagggaggtaatgataaccctcactctggggggctgtttggagggttaaatcagttattgctgtagcatgcatttctctgtcaggtattgagtgaggtgctgtgattttagccctgcatttttcttttcttaccattcaataataacgttttgagcacccactgtgcgccaggcaccatattaggtgctggggatacaaatgtgaatgaaatgaatgtggtctcttcccccaacagtgtatccagaagattaatccattccttaaacaaatgctacttgacacagattagttctggataggctgagagctctgaaggagtgcaggcagctgcgagcctgtgtatccagcagaaggatcaggaaaggattcctggaggaagcgctgttctagccaagacctacgggggcattattaaccaggcaaaggggacggtgtccaagcagtggaatgaacgtggattgaagctgtgaggcaggagggagtgtggcctgtgcagaagggaccgaggctggtgagaccaggagggcctgggtggcctccaggtcagatgtgaaaggaagaacttggccacagtctgagcttctcaggcgtatggcagggctgcctggtgagagggaatgagctccctgctctggaggtatgcaagcaggactgggctctcacctgccagaggccacagagctttccagaggctggaagaggccactccaaggcctctttgcccctgagagtggtggctcttcttgaggccaccttgccacgctgtcacagggaactagcagcccctgcctcacccgggggtttggaagatagagggaggcctaggaagggccctgtgtctcatccgagctgggcccctttccagcctctcactggaaggaagcccaaggatgttcctgtgggggcttttaccaggcccacctgccctctgctggccatgcttgcagcctcctgaccctgtcccagcaggacagtgggctggtgtgagcgggcaggaaccgcctgcacttagaaggtgtggggctgcctccccgagcttccatctgccgctggggccacaccccaggcccagggatgggaccccacagtggtcacatcatcttgcagcagaacccaggtacagctcctggagcagatggtggtcccaagcacgggtgggaccagaaaggactctcacctgggctaactcagctgcagcctcagttccctcctcacacacgacgaggaacatggactggaagcctgcccagcaggccttctgctcgatgtgcgttgtgtggcttacgtccagggagggaagcagcctctgtgctgtcttctagataagcctgtattccccgggctgtctgccaatgtatccagttgtcccgtcagcctggaagctctgagggaaaaccttgggctgcttcctgagcacctgtatcccctgcagccagcccggggcctctgctaggagcagactgagcatggcttatgggcctggcaccatctggcctctgcccaccttgctggccttgtcttgtgtctgccccttcgacattccatagcccagctcaatatctagtggttcctctagggtggcgagcactgtttggtctccagatgtcttcaggtcggagctcacagcgctctcagccaccccttcccagtgtagcaccgggcacatggtagatgcctattgatgagtgaaagctcctaacacactcagagagcaaggactccgcctcatcccacagcctgggaggagaggcagactgccaaggacctgctcagcatgctacagaagaaaccaaagtgcccacgggactgatcagtggagcttcctgccgagactggaggccttagggcagggtagacagtgtgtgtgcaggctggggactcacagttcggactgtgcccagacctactagcatagtgggtgggtgggaggatgcgggactgggggccgaccttgcctgaaattcatgtgggatctcagagcagccactgaattgctctgtagggggctaaatagtggcccccacagatacacacacccagacagagcctgtgagccagaccttatttggagaaaaggtctttgtagatgtaattaagcatctcaagatggcatcatctggattatgcggtgggctgtaagtcctgtgatgtgtctttatgagagaaaggcagagggagatttgacacacacaggaggggccacgtggagacagaggtggagattggagaaatgtggccacaagccagggaacaccagcagccaccagaagccggaagacgtgaggcagggttcttcccagagccttcgctgctgagtctgggaatttgtgaccgaagccataagaagtgggtacacgccctgagcctcccacacttgctcacctgtcctgagatgagaatctctactctgcagcatatttggaggatcactgcgggggccacagaggtgctgttcagatggcacttcagaagactcaggagaccctggggcaggagcagtttgactgacagcccagagggctgccctctgattccacctgaggccctgcttttcctggctgcaggggttccagggccaggccatttccgctggcgcaggactctgctagcagcaacctgcctgaagtcttcctttggcctggctgagagtttctgagacctgcgctggagcggaggtgcttccttccttgcttcctttcttcctctctcccttctccatccagcaggctggacctgcctggcatctgtgagctctccctactttctcctataccctaacctttgtcctgcatgggcgactcccccagtgagtctcttgcagcttttaccccagtgcctgcttcttggagaatccaaactgatccagttagggatgataaagtgtagggtaggcgctcggtgactgttttctctgaggttgtgactcgtgtgaggcagaagcagtccccgtgagccctcctggtatcttgtggagtggagaacgcttggacctggagccaggaggcccagacatacatcctgtccgagctgcagcttcctgtctctaaaatgagccggccagcgcaggtggccagacatcactgttattctcctttgagtctttaaatcttgttgtctttcttgcagactcggtgagctgtgaaaggctataataggggctttattttacactttgatactattttttgaacattcatattattgttagatattgatattcatatgaaggagcaggatgacttgggtccttcttggcagtagcattgccagctgatggccttggacagttacctgccctctctaggcctccctttccttgtctatgaaatacattatagaataggatgtagtgtgtgaggattttttggaggttaaacgagtgaatatatttaaggcgctttcaccagtgcctgggatgtgctctgtagtttctgtgtgttaactataaggttgactttatgctcattccctcctctcccacaaatgtcgccttggaaagacggaggcagcctggtggaggtgtatctcctagacaccagcatacagagtgaccaccgggaaatcgagggcagggtcatggtcaccgacttcgagaatgtgcccgaggaggacgggacccgcttccacagacaggtaagcacggccgtctgatgggagggctgcctctgcccatatccccatcctggaggtgggtggggactgccaccccagagcgttgcagctgtactcctgggttgcaccccccccagctgtcactgtcccctccctgccatcagttgtgggaagggcgttcatccatccagccacctgctgatttgttatagggtggagggggggtctttctcatgtggtccttgtgttcgtcgagcaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggtaagtgatggccccagacgctggtctctctccatctggacctggcctgggaggtggcttgggctgggcccagggagagctaatgtctcctaaccaagaatgctgtggcagcctctgccgcagagccagagaaccagagtgccaaggctggcagggttcccagtggccacgagtgcagatgaagaaacccaggccccaagagggtcatgcaggtagcccagggagttcagccttgaccctgggtcaatgacctttccacagttccacactgctccccttttaaaatccggtgatgtctttatgtcttttgttatgttatcttcaatgtggagggactcgaggtgatctaagcaaactttttctatcttctgcttgcatacctctgagaccaggggactcactcacttgcatgactgggccctgcaggtcacactggccaggcagatgtggtggaggaactggcagaggactttttctagactgtgactacatttagtccacccagcggcccccctatgaagtccagttgagaactaggactctgggggccggtggacagagaagagggagggttctctcccttactgacttccttctgtggccagacattgagcaaggcctctgtacagcatgtcctggggctggccttgccgtagctgctaaatagttgacgaaaccagtccagagaggggaggtgactgccagggtcgcacagctcaagctggggaactcgctgggaaaactgtcagctctgggcagcagcttgacttccactgtaagccccagcccccagggtcaaacactggctctggtgctggcagaggcagcccactagcctgtttcaaaggctgagaaggcccaggagtctgccctgtgctccaccagttctgccctgagactttcctacagagtacaggttttgatgttcagttttaaaggcaagaatcaataaccttctgccccatcaggtgaccccttgtgcctgtcccacccctttattgactgacctcggctcagtcaggtcagttcctgaaggtcagtgtgtggaggggaggctgttctttcccagaaaggccttccccaggcctggtgctctggcctctggaggacttcctggagaagtcccttctttggggtcccagtcagtgtatgggaagcccttattgcatgacctggcacggggcaggggctcaacagtcactattgccttccttgccactgccatttcctcctctgtaagcaggtgattgtgtgtccagtctgagcacagagataagcacacagcaggtgcttaataactagcagctgtaggctgggcgcggtggctcatgcctgtaatcccagcactttgggaggccgaggtgggcagatcacctgaggtcaggagttcgagaccagcctgttcaacatggtgaaaccccgtctctactaaaaatacaaaaattagccaggcatggtggtgggtgtctgtatcccagctacttgggaggctaaggcaggagaatcgcttgaacccaggaggtggaggttgcagtgagctgagatcgtgccactgcaatccagcctgagtgatagagcgagattccatctcaaaaataaataagtaaataactagcagctgtaaatgtggctgttgttcttcacctccacactcagtgccactccactccctccctccgtggtgtgaggggcctcactagctgtctcctaggaggagcatggctgtgagattccagctccatccttggccacggctcctggagacatcttagaggccaggatccagaaggctcccacacctcatttgacaggggagaagctgtcagttccaggtccccttgcacatcagggccagagctgcgttaggcctccagtctccaggccactgggccagagctcacaggctggcagagggttagaactgttactggtggctgggtgcagtggctcacgcctgtaatcttagcactttgggagggcaaggcgggaggatcatgaggtcaggacatcgagaccatccttgctaacacggtgaagccccgtctctactaaaactacaaaaaattagccgggcgtggtggcaggcgcctgtagtcccagctactcaggaggctgaggcaggagaatggcgtgaacccgggaggcggagcttgcagtgagccgagattgcgccactgcactccagcctgggcaatagagcgagactccgtctggaaagaaaaaaaaaaaaaagagctgttactgttgacagtagcatgaggtagaccatggcctgcaccaaaatgggggagtggagtgccactgaggccagaaggaaccacaccctcaagggtggggagttatggtatggggggtcctaggcatggagtcttttaattctttagacaatcctgggagcaactgtccctgtttcacagagggcggggccacacagctggtgagtgggcagccaagactctgttcaagtttgtgtgggtccaacacttgcggccacggtggaggggcatctgagccaggcctcagagagtggcgggggaagttgggtggggaagtgtgcccttctcattcctctgaggctcatcctcttggtgcctctctttcatggaaagggataataaggttattgtgaggatcccctgagttcgtatattcagacgcttagacagagccaggcacagagaagggcccggggttggctagtttgattgctggtgtaattgctaatatcttccagtttgtattggtcaaggttctgcagagaagcagaaccagtaggatgtatatattaagagtttcaagctcatgtgaccgtgcgggctggcaagtctgaaatccgcagggcaggccaggcaggctggcaattcctgcagaatttgatgttgcaatactgagtcctaaggcagtcctggggcagaattccttcttccctgggaggcctcagtctgttctcttaaggccttcaactgattaaatgaggcctgcccaagttatagagagtaacctgccttactccgtcttctgatttaaatgttagtcacatctaaaaaatattttcgcagcagcatttccactggcttttgaccaaacatcaggccacaaagttgatccccaaaattaaccatcactctgtgcctgtaagggaggggctgggaaaggggagcaggtctccccaaggggtgaccttggctttgttcctcccaggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtaggtgctggggctgctgccccaaggcgcgggtagggggcggagggcggagggcggagggagggcgggcgggcaggcgggcttcttgtggcacgtgggcttcttgtggcacgttcctggaggccgaacccttctggctttggaaggagtcgtcagagacccccgccatgcgggaggctggggaggaaggggctcgaaacctccatcatcgcagagtctgaatagcagtggccccgccatgcgcccacgtagcggcgcctacgtagccacgcccccacaccccgtcctggccactctccctcctgaaggtcttctggtacccgccccctccccatctccatccccaggccctgcgtcctctgcccaatactctttgggcctccctgttgtccagctctctccgcggctccatgactgacaacttgagcaaggctaatgtgaatgggagcggttgagggctcagacctctcacccgaggaacatccacagagtgtgccgcatgcccggtgcagtgtggctgcggggacacagacacggagcctcggccctgaggagctggggggcagtgaccgtccctcctctgacccaccactcctccagtgtcaggacactgcgggtatctaggggaaggaatcttgttccacttcaagtctggaacttcaagtctgtgtgtgtgcgtgcgcgcgcgcgcgttgggggtgggggttgcagagcagatgcgtacctgacagcggtaacctaggtcccccctggcctatcaaggcttccctggcggccgaatttaaaggcatcaagcaaacaaagcccaacacatctctgccttgtcctctcagtttccccccgtggcacttagaaccacttgatacaccgaatagtttcctatctcccccactaggatgtaaactccacaggggcattgggaatgctgcctggctatggtagggacagaggggagcaccagggcggggcaggggtgccagagttctgcctgggcagtcagattttccttaggaggggacatttgagtgggacccaaacaggtgtatagcagttgtccagcccagctggcaaggcctgagtctgcctctgcaacccctctcttgggctcctttctctgccacccacctcctcacctttccaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggtaagtcaccaccccactgcctcggccaccgtgatgctaacagcccctttggcagtcagggtctgtgccgggacctccagtgccaggctctgtgcagggggaccagagatgaagtaggcctgatggtgccttcaaggacactcagtctgatgagggaggcgagtgcacagaggaaacacgaggtcagggctgtattagagggagcccagaggaggcacctgcccagcccgagggtcagagaaggcatcttggaggagggacatttgatcgggagcttgatggatgaataggagttcacctggccgataagacagcaactaccaaggcttagaggtgtgagaggaggctgtcttacctcactgagtaaggactgcaggcggcttaccttcgagaagagagcttagtgtctgtgtgcacgtgtgtttgtgtgtatgtgtgtgcgtgtgtgcactggcaggagtcccctgctggggcaggagggccgggccatcaccatctttcaccattcacccctgcaccaggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggtaagcaggatggcagggtgggcaagtccaggctggggcttgggaggtctgtgtgaccttgacagtctctcccttctcccttgtctgtgtaaggaggatgacgccaccttaaataggattaaatgagaatggggctctgaaagggctgtgcaatattttcataacgtgtttttatagagacagttgagtatgttctttaagccctcctctctcctaccatgaactaaagatttctgtggaggtcccctcactcccagcaccccctcctcatcccaggccctttttgcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggtgactgtacccctccttcgtgtgtgtgtgtgtgtgtgtgtgtgtgtgtgtgtgtgtgtgtgtgtgtgtgtcagtgctgggccctcagggacccccagcaagcccctccatcctccagactccagctcttctgtaagcttacagggctggccagaccaggagtggggcactcctcacttcacgcggctgggggctgctggagagagccacagcgggaagggtttcctagaggctgcaggacagtgctggatggattttcaatgctcacctgggtgtgagcgtgcggcagggccgcgtgagggtcagcgatctgctactctggactcagccatctctaggcccctctcactcaggtgctccatggttctgggagctgagaaatctcaaaccagcaaaaaagtggaattgatgttgatgctacaggatagtgcacagatgccatctggttgcagcattttggtggaagggcagtgcccagctaggagagtgaggaggggcaggcatttctggcttgaggagatggggtcttaatgctcgtgtgagaggcagagtgggtggagtggagctggctggatccttgctttggcctcctggatttctctctatctccattttgaaaccactctgtgtttggaagaacttttgagtattcagagctgcccactggcagaacagtcttccttgggcaggagtgagctccttgtccccagaaggctgggtctggctggcccctggcagggacactgatgagggtgcttgagttgatcctgtctagtccctttctgtgttttcaaagcccattctaaagcagattcccatttccgtctttgactctaaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggtaggaggctgggcttgccctggggtgaggaggggtctctttctccttatgcacccactgcccgcgaggcttggtcctcacaagtgtgatccatgagactcaagcctgacttgcagttccatactctggttctgccacttccatgccctttgagcctgggcaggtgaccttacttctcctcatctcagcttcctcctccataagagggaaaaaggtattacctgcctcattgtgttgcaaggagatgggcagcatctagggcactggcctggagtatcgcaggtgctttgcctaaggtggtgcagtccaggagaggcagctccagagagaggcccccggctggggctgaaaggagggcagacctcggtttgaatttcaccctgccgctctatagctgtgtgacttgggcaaattacttaacatctctgtatgaggaaatgatgagtgctaagcacttagcttagtgccgggacaatataaattctagctatcgttactattgttttcatcacccgttgctttaaaatccagcctctggtataggcaactattgacgggctaccctgtgtcgaaaacatgcccaggcaggtagcaggaagtcacagatggggacctcttggggcatcaagggatggtgccctgaggctgagctgttctggttgggtggagcatgagaggtctgggaagacagtgggactccagcctggaataagaggctcagagttgattctcgtctgagcacgtccaggggaaccactgagggtttgggaacaggagagtgagggtgagaacctggttctgggcacagcaggctggcatgtaggatggatgttcaggaaagatgagcatagtcaggtggctggtgcccttgtccaggggagaggctccgtcaggttcaggggtcctggcttggagggaagtccgccatgctctaatcacgctcccctttggaagtgctcagccgatgagctcacaggcacatgtcagtttgaagtcatggaatctgactccatgaagcgcacctcaaagagcaccattttgcagctaagggaactgcaggctggacatgctgagtggctgccccgagcccttgcagctaggacatagagaatgctagtaaccacaaccctaccatgttcagagcacatgccaggctccatgctggggcttcgcacgtgtcatcttcacagtgtccctgtgagtaggtgtggtttctctttccatcttacaaatgagtaaacagagcctcagtgtagctaagtaaccactattttaggtttcttagccaatgggtgtgtctgactcctaagcccatggagggcattctgaggtggttcagacagaccccggcttacccttgaacttctgcctgctggctgcatagggaggggctggggggagtttgagcatctcaggccatagagcccctgcctcactgtctccatctctgggtggaaagatggtgttttccctgagaaactaaggctcagagaggttgaatggctctcccaaggtcacacagctggtcagctgcagagttgagaacacaggagtcctggtgctcaggccagcatctctttttttctttgagttgtttctaggtttcctagctcttgcctcagaccttaaagagagagggtctgatggggatgggcactggagacggagcatcccagcatttcacatctgagctggctttcctctgccccaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaagaggcccgtgaggccgggtgggtggggtgctgcgtgtctctcctgcacagcttttctgtgtcagtttgtgccaccaccataccgccatgcatcagggtggcggtttgccaggtagatgctgtgggcagcttccgccattgtgtggacagcatgtatatgtgtctctgtgtggctgggtctgtttttgcttttgtccagatcagtaaggtttgctacctgggtaccccactccacttggagtagaatgtgcataaatatggcataaagaaatgcaatatgcatgcatttattgattgatctatttttttctgagatggggtcttgctgtgttgcccaggctggtctcaaattcctgggctcaagcaatcctctggtctcagcctccccaagtgttgggattataggcatgagccgctgcacctggcctctctgatctatttaacaaacctgctgggagggtctcagggtcaggagcagcactgggctctgaggacacagagctcactcagccgtgacccagagggggtgcctgagctgcatgctgaaggttgttagcatgaccagcaaggcaagaaaaggccctgccgagattagcaaggcatgtgccaagccctggaatgtgacagccgggccttctagaaacctgagtgtataactctccttaaaagccagtaggagctcctcaaaaggcagccctaaggagtccactcttaaatgaactcagagtcagttttaaaatgcaagtctgtgttgattctggtctggatggtgcattcctcgagagcaaaagacagtcttggtcttggatccacttgccctgggtacactgagggctgctaggttccaggtgctcttcctggcactggggagggatacaggcccaagagacatgctgttctccctcctggagcatctattttagtggaggaagacagaaaacaaaccattaatatagagtactgaaaagatgcgatggagaaaactatagcaaggaagggaatggggtgggagagaggtcaggagaggtctcgctgacaaggtggacgaaacaggccatgaggcagagaacatgttccaggcaaagcaaaggcccccaggtggggatgtgcagggagtaccaggaaaccagagaggtgggaatagttatgagatggggggtgcctcagaggggacagggccaagtcaggtgagacctgagggtcacagtcagcagtgagctggggccatgcaggggtctggcctcagaggagtgtggtctggcctggatctgaacctctcactgtggcctagctgctgagctgagaagagatgacaaggaccttgggcagaagcagggagactggagggaggcggtggagggtccaggcgttggggcggggctcaggctggagtctgaagggagcctgcaggcctggtgggtggatgtgggtgggagagggggaggatggcaccaaggctcgggcccctggacagatggagttgccattaagtgggatggggcaggctatggggccatcagtttcagagggatgagtttggcactggcatggtaggcatctgtctatctccacggccctcaaaccaggcatgaagcaggagctcacgtgtttggtcagccatggtgcagaaccgcctgggtgggaggtgcggggtgggagatacacggttgtgtcccaaatgggctctgagccagcgagggccgtctgcactttggcctcacagaaggatgtcggagggagaaatgaagtgtgggtgggggtcccgggccacgctagacatgtgctttcttttcctcgggctctggcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa

SEQ ID NO: 2 - DNA, 25305 bp, Homo sapiens
  source 1..25305: mol_type = genomic DNA
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgctttccgaggaggacggcctggccgaagcacccgagcacggaaccacagccaccttccaccgctgcgccaaggtgcgggtgtagggatgggaggccggggcgaacccgcagccgggacggtgcggtgctgtttcctctcgggcctcagtttccccccatgtaagagaggaagtggagtgcaggtcgccgagggctcttcgcttggcacgatcttggggactgcaggcaaggcggcgggggaggacgggtagtggggagcacggtggagagcggggacggccggctctttggggacttgctggggcgtgcggctgcgctattcagtgggaaggttcgcggggttgggagacccggaggccgaggaagggcgagcagagcactgccaggatatcctgcccagatttcccagtttctgcctcgccgcggcacaggtgggtgaaggagtgaatgcctggaacgtactgggaactgcaccaggcacagagaaagcgggcttgccattatagtgggttccgatttggtttggaaaacatgggcagcggagggtggagggcctggagagaaggccctacccgagacaggggcggggtgggaaggacggcagatgctgggagcacgaggcaatttctttatgacacagaactcatgctctagtattccatctgtttcagccgaagaaaagaaccagctgaaggggcaggggagaaggggcggaggtattctcgaggcccattggcgtcctttaggactcaggcagggaagggcccttggtgctctggagccggaggtggtgcgcctggtactgggaccccggagctgagcccggcgcctcagcccacctggctgtctgccgaccgtgtgcggggcgagtttgctcaacaactctgccagcttctggccctcaggctgtgggaagcttcttcccggggcgagaccactagctttttctaagtattaccagcccaggacttggctgaggttctgtgtcccccagcttggagtcagatgtggggttgaatcttggcttcctctcactagctgtggtgcttgacaagtcacttatccttgagcctccattgcctaatctttaaaagggaggtgacaatcgtccctacggctcagtggcagcagatggggagatgaagggaaagttctgttgaccatgagtgaacttacaatgcaagccccggggggatcacttgcagttttgtccctgtctgcagtgtgacctgttggtgacattgtctttgctccaaaccacagctcctggggcagaggggaaaattctgccactcacagctgcctgcccacgcttctgtctgagtgtgctgggtggcaggatggcaagtccttactcagctcagtatagccctcttccttgttccctgagcctttgactttctcgagggatgttgtggggttgtggccaggataagaaagggcatttcaagttaccactgctccaaaacaactgttctggaaatagtgagtaccccatcctgagaggtgagtaagcagaggctgtatgaccacctgaaccaagcccttgaggatgtttcttctctggtggaagtttggaacaggagcctcctcaagttcatttattcattcattcaatggttattttgtgggaatcgaatttagaatgaaaatattttttggcaagcagaaaataatttttagaccaatccttttcttttagtcatgagaaactgaggcccagagagaggaggtcaccccaggtgcattagaactgggtttccagaactgacactccactgcacagagtactctcccaattcattcaatttttatttagcggaaggcattttcagatgggtctttgaagcattagtaggagttcagcgatgatggtgtcatgagaattttattctaggattaggaggtaccatgaacaaagatacagagctgggaaaaccagaggtggaagataaggagcacatgtccacagttctttttcttttttttttgagatggagtttcgctcttgttgcccaggctggagtgcaatggtgcagtctcagctcactgcaacatctgtctcccgggttcaagtggttctcctgcctcagcctcccaagaagctgggattacaggtacctgccaccacgcccggctaatttttgtatttttagtagagaaggggtttcaccacgttggccaggctagtcgcaaactcctgacctcctcagtggatccgaggaggtgatcctcccgcctcagcctcccaaagtgctcgaattacaggtgtgagccaccacgcctggcctccacagttctttatccaccgtctgaaatgtaaaatgttacgaaaaccaaaagttttttttgtgatttatttgatggtagcacctgacgtgaactgacatgagattatttttaatttagttgtgtgaatatgcatattcatatattttgctgcatagattacagtatgcagctccagattcttccaagcagactctgattgcccattactgcctttctaaaatccaaacaagttctgaggttcaaaaccgttttggccctaaggctttgggtaaagggggtggactctgttctactctgactggagtccaagatgcatatatacagagatatgggtgatggggctgcaaggtaggttgaggtaggggccaaggaggagcatggagtttggacttgattcatgaggctgtggggagccagtgaaggttcttaagcaggtatgtctgcctgagagcagttggagcagacaagagctaaaaaccaaacaaatcaccatagatagtggctgctataatttgtttgtcccctccaaatctcatgtggaaatttggtcctcagtgttggaagtggggcctaatgggaggtgtttgggtcatgggggaggaacccctgtgaaaggcttggtgccgtccttgtgataatgagtaagttctcccgctatgatttcccttgaaggctgattattaaaaagagcttggcacctccctctcttctctcttgcttcttctcttgccatgtgattgatctctgcacatgtaggctccccttcaccttctgccatcagtgaaagcagcttaaggccctcaccagaagcagatgctggtgccatgcttcctggagagcttgcagaatcatgagctgaataaatcccttttccttgtaaattactcaccttcaggtattcctttatatagcaacacaaaaggactaagacagtggccttgacttttctctctctttaagaagtgttgcctttgctcacttagtcatcccttctgcctgcatttgtagagcatctggatgggagatttatataaccgtcactcttgactttcccagcaggcctatgtcataggtactgtggtctctacaatacagcagaggtatctgaggctccgagaggttgagtgacttgctcatggctgcacaaccagtaaatattggagctggaattcaggtccacggtttcctggctccaaagcccatgattttttccctcaatttattctgactggggcatgggggagggggtggcctttgggcagggccaccaggagcgaccaggcccgtagagagctgggtgcaggtacagaggaaaacctgttgtcgagtgtggcccgtagttcccatttttgcctgaatggcacatttgaaagtgttatataaccatgtgaataataatagttggcctatatgagttctttaatttgctttttggtccgcatttggtaacttctttatcatctactatactctgttgtgtctcttttgttgtaatttgtaagtaggggtgagataaagtacacctagggtttgctgggtttcttccatgtcatcatgttcctccttgcatggggccaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggtgagccaccctttttgggaatggcacttcctgatagggctgggccactgcatatacactggggactgtgcttagtaggcccattgctgaaaatcagaaggggacagcaagtatgtattgagcacttatcgggtaccaagcacagtaactactggctttctgtatagaattccctttaagcctggccatgccccagtggtacgtctatcttcatttgaaagacgaggagactgaagttcagaggggaccacacagacagctaggggtagagcctggatcaaacccattggtctgcctgccagccattcttgtgccaatgcatctgctgcctacggaaacctgtagggacaaggccctgggatgttcagtggagcctgagtcattttataaaaaagcatgactctagggtccaaaattcctttgaagctgttgctatccagagtgaagtcccttctttaggacagggtggccctcctccctcctggatgtcacatcttcggtggaggggcagaaaggggactgggtattctcctcaccctggccctagtgcttcaaatcttaaaaaaacgtttttatttgtgcttctgcaccaccttctagcccacctcgtttcctggcctctaacttgatgagagcgtgtgtcattttcacactgattctccacatggcaggcggtgcttcttagcctcctgcagacagtgaggccccacggtcttgtccaaggtcacacagcgtgtaatgggcagggtcagagtctggagtctggacctgggtctcctagctgcactgcactgctgccccatgggttaatcagctcagcataccgtggctgaacagctacctcataccaaggcctgtggcgccatgacagggattgacagggtccctgccttggaaacccgtagtctaagtagaggagactgacaagtcaatgccttccatcagtctgctcaacacacgtttaccaagtgcctactgtgtgctgcagaggcgaagatgacacagctcaggcctttcccttgagcttacagttcaggaggagagactgaccagtgactgccagtacagttgactatgggacaatgtgctcagccttggggagagacgaagaaggtacccgtatagcaccagatgacaggcacgagccccacaggccagggcagctgctcagaggagagtaggccaagcagaaggcaaacagaaggctgcaggcatttgccatcgagagctggacttcaaactgggcatcataccagcctgggttcgagtcctgcccagccccttattggctgtctaaccctgagcaaatcccttcacctctctgagcctcattcctctatctgtaaaccagttataataattggaacattcatttaaggactaaatgaggtcgtgaagcattcagcagatgctaggtacggaaactcgctgaagtgggggcaggttaagaagcctctggggatacgaaggcatccagggactagttgtggcaggaggctgttaccacttaggtctgaagggtaaggagagggaatagctttccctctgcccagttggagccggtggcatggaggagaggctgcctgtggggaatcacccgagggttcaccgctgccatgcgcagggagtcaggaggtagggagggagtggggcagatgcacaccatttttttttttttttgagactctgttgcccagactggagtgcagtggtgccatatctgcacctctgcctcccgggttcaagctcactgcaacctctgcctcccgggttcaagcgattctcctgcctcagcctcccgagtagctgggactacaggtgtgtgccaccatgcctggctaatttttgtatttttaatagagatggggtttcaccatgttggccaggctggtctcgaactctcgacctcaggtgatcccccacctcggcctcccaaagtgctgggattacaggcgtgagtcaccgctcccagctgctgatgcactcttgtccttctaactcctgctagtgcctcccattggctgagcccaactggaagctttgcaagggagctggtgctgcagtttgcactgagcaggctggagaaggctggagaatagactaggggacaaaccgaattgccagtgctgttatgtcatgatttaggcatggagtccagggcctgagcttcactccatgtccatcctgcccagagccttggcacagcctggctcccagacaagatgtcaagttcagaatccttcctaaaaggaatcctctatgccagaccgtgttgcagggatatgggagtgctgggctcccagcctgatcaaggagcgagaaaactcaggctcctagtctgtcctccggggcactagcagggacaaggtgggaggctgctgggctgggatgtggggacaggtttgatcaggtaaggccaggctgtggctgtgtttgctgctgtccaaatggcttaagcagagtcccccggcctctctggcttctgcaggccttgaagttgccccatgtcgactacatcgaggaggactcctctgtctttgcccagagcatcccgtggaacctggagcggattacccctccacggtaccgggcggatgaataccagccccccggtaagacccccatctgtgccctgccccaccccatctgagctgaatccatttgctctgccctggcctggcctccctgctggtggtttccacttctcggggggctttgggactcagcacctccactgacccctttttttctgtcccatccccatcccctgcagcccccactgcctgccttcctgttgccccacaaatgcaaaagtcttgccttaaatgatcctcttttccttcttttctcttgttttccttttctcaccatttggaatggcccagcaggctgcacttaccttggaaggagggttcatctgatggtgactctacctagggcccccaggcctctataactcccagtgccctgcagactggaccagatcctttaatgggatagacacaaccctgtctgggatgcctctgcctaccttcctgttttgctgctccacctgcctccagctccgtttggcttcctggggctccctgcctgggccactttgtgtcttccctctaggcctttctttccactgttccctctgcctggtgtggcctggctatggaagggagggaggaggagcggccatggaaaacggtctgcattctagcagggacttgcaggtggcaattcagtcggggaagactctagatgcacctggcctgaggagagaatgaagggttctagttggactgtgttaagtttgaggtgcccatggtgtgaggtctggagctcagcgcagagatgatgcaatgtggtgggtccatgcaacatggtgccaggacgcagagcttggggtgaactcagctttcaccccttaccggttctcgtgggatcttgggaagccactttcttctatgagctttgtcgttcttgtctgtaaaatgggcacataaccctgtccctgtccttctcacaggttgctgtgagactccaatgagttgaaggatgtgcagatgcttttggaagtgaaaagttggggggctactgtgtgactttgcatacacccaaactgtgtgaccttgcatatgtctgagttgctgccattgcaacagatcagagctggtgggctgggtgtggagaaagggtttgtgtgggggacatcctctggcaagggtggcagcagcagaagtgaggggcctggtcggtcatgtgtgctgacccggcctgggcagcctgtggccagggagaggacagctcctctgtaggaagagcctgttcctttccaaccaggtgagacctcttcagtggagccctggagccccctgtactccacatcagtgcctcagggacctcccggagcaggctaatatcagagaccaagagggacactggcagaggatcacagagaccccagtccaggcagggactgagaagatcttgccccctaagttagtttcctagcactgctgtgacaaattaccaccccctcggttggaacaagttgattctctgcagtcctggaggccagaagcctgaatcagtgtcggcaggaccactttctcccggggggctccagggagaagcttctcttgcctcttccgtgtcccaacagcggcagcacaccaatcccagcctctgtcttcacacagccttctctgtgtctctctcctcttcattgtctcataaggacacttgtcattggatttagggcccactggatcctccaggatgatctcatgtggggaaccttaaccacatctgcaaggaccctttttccaaataaggtcacagccacaggttgtgggggttaggatgtgagtgtatctctttggcagccactgttccctcctctcccttgggccagaagcagacgtggggccctttcttccccataggatgcccatggattgccccccttcccgcttcccccgagtgtctgtgggaggtggcaggaatggcaggcaggggtgtggaaccccttctggagtcatatcaagggcttggctggaggaagtcctcctggagctgttgggctggcatggggcaggctggctgggcccagcagcagcttcttcattcatggggaggccacaagcatgggccctagagctggctgccgccctcaaacccagaccctgcactcttaactgtgtgaccttgcatacgtcactcaccctctctgatcttcaggttcctctgcaaaagggaggtaatgataaccctcactctggggggctgtttggagggttaaatcagttattgctgtagcatgcatttctctgtcaggtattgagtgaggtgctgtgattttagccctgcatttttcttttcttaccattcaataataacgttttgagcacccactgtgcgccaggcaccatattaggtgctggggatacaaatgtgaatgaaatgaatgtggtctcttcccccaacagtgtatccagaagattaatccattccttaaacaaatgctacttgacacagattagttctggataggctgagagctctgaaggagtgcaggcagctgcgagcctgtgtatccagcagaaggatcaggaaaggattcctggaggaagcgctgttctagccaagacctacgggggcattattaaccaggcaaaggggacggtgtccaagcagtggaatgaacgtggattgaagctgtgaggcaggagggagtgtggcctgtgcagaagggaccgaggctggtgagaccaggagggcctgggtggcctccaggtcagatgtgaaaggaagaacttggccacagtctgagcttctcaggcgtatggcagggctgcctggtgagagggaatgagctccctgctctggaggtatgcaagcaggactgggctctcacctgccagaggccacagagctttccagaggctggaagaggccactccaaggcctctttgcccctgagagtggtggctcttcttgaggccaccttgccacgctgtcacagggaactagcagcccctgcctcacccgggggtttggaagatagagggaggcctaggaagggccctgtgtctcatccgagctgggcccctttccagcctctcactggaaggaagcccaaggatgttcctgtgggggcttttaccaggcccacctgccctctgctggccatgcttgcagcctcctgaccctgtcccagcaggacagtgggctggtgtgagcgggcaggaaccgcctgcacttagaaggtgtggggctgcctccccgagcttccatctgccgctggggccacaccccaggcccagggatgggaccccacagtggtcacatcatcttgcagcagaacccaggtacagctcctggagcagatggtggtcccaagcacgggtgggaccagaaaggactctcacctgggctaactcagctgcagcctcagttccctcctcacacacgacgaggaacatggactggaagcctgcccagcaggccttctgctcgatgtgcgttgtgtggcttacgtccagggagggaagcagcctctgtgctgtcttctagataagcctgtattccccgggctgtctgccaatgtatccagttgtcccgtcagcctggaagctctgagggaaaaccttgggctgcttcctgagcacctgtatcccctgcagccagcccggggcctctgctaggagcagactgagcatggcttatgggcctggcaccatctggcctctgcccaccttgctggccttgtcttgtgtctgccccttcgacattccatagcccagctcaatatctagtggttcctctagggtggcgagcactgtttggtctccagatgtcttcaggtcggagctcacagcgctctcagccaccccttcccagtgtagcaccgggcacatggtagatgcctattgatgagtgaaagctcctaacacactcagagagcaaggactccgcctcatcccacagcctgggaggagaggcagactgccaaggacctgctcagcatgctacagaagaaaccaaagtgcccacgggactgatcagtggagcttcctgccgagactggaggccttagggcagggtagacagtgtgtgtgcaggctggggactcacagttcggactgtgcccagacctactagcatagtgggtgggtgggaggatgcgggactgggggccgaccttgcctgaaattcatgtgggatctcagagcagccactgaattgctctgtagggggctaaatagtggcccccacagatacacacacccagacagagcctgtgagccagaccttatttggagaaaaggtctttgtagatgtaattaagcatctcaagatggcatcatctggattatgcggtgggctgtaagtcctgtgatgtgtctttatgagagaaaggcagagggagatttgacacacacaggaggggccacgtggagacagaggtggagattggagaaatgtggccacaagccagggaacaccagcagccaccagaagccggaagacgtgaggcagggttcttcccagagccttcgctgctgagtctgggaatttgtgaccgaagccataagaagtgggtacacgccctgagcctcccacacttgctcacctgtcctgagatgagaatctctactctgcagcatatttggaggatcactgcgggggccacagaggtgctgttcagatggcacttcagaagactcaggagaccctggggcaggagcagtttgactgacagcccagagggctgccctctgattccacctgaggccctgcttttcctggctgcaggggttccagggccaggccatttccgctggcgcaggactctgctagcagcaacctgcctgaagtcttcctttggcctggctgagagtttctgagacctgcgctggagcggaggtgcttccttccttgcttcctttcttcctctctcccttctccatccagcaggctggacctgcctggcatctgtgagctctccctactttctcctataccctaacctttgtcctgcatgggcgactcccccagtgagtctcttgcagcttttaccccagtgcctgcttcttggagaatccaaactgatccagttagggatgataaagtgtagggtaggcgctcggtgactgttttctctgaggttgtgactcgtgtgaggcagaagcagtccccgtgagccctcctggtatcttgtggagtggagaacgcttggacctggagccaggaggcccagacatacatcctgtccgagctgcagcttcctgtctctaaaatgagccggccagcgcaggtggccagacatcactgttattctcctttgagtctttaaatcttgttgtctttcttgcagactcggtgagctgtgaaaggctataataggggctttattttacactttgatactattttttgaacattcatattattgttagatattgatattcatatgaaggagcaggatgacttgggtccttcttggcagtagcattgccagctgatggccttggacagttacctgccctctctaggcctccctttccttgtctatgaaatacattatagaataggatgtagtgtgtgaggattttttggaggttaaacgagtgaatatatttaaggcgctttcaccagtgcctgggatgtgctctgtagtttctgtgtgttaactataaggttgactttatgctcattccctcctctcccacaaatgtcgccttggaaagacggaggcagcctggtggaggtgtatctcctagacaccagcatacagagtgaccaccgggaaatcgagggcagggtcatggtcaccgacttcgagaatgtgcccgaggaggacgggacccgcttccacagacaggtaagcacggccgtctgatgggagggctgcctctgcccatatccccatcctggaggtgggtggggactgccaccccagagcgttgcagctgtactcctgggttgcaccccccccagctgtcactgtcccctccctgccatcagttgtgggaagggcgttcatccatccagccacctgctgatttgttatagggtggagggggggtctttctcatgtggtccttgtgttcgtcgagcaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggtaagtgatggccccagacgctggtctctctccatctggacctggcctgggaggtggcttgggctgggcccagggagagctaatgtctcctaaccaagaatgctgtggcagcctctgccgcagagccagagaaccagagtgccaaggctggcagggttcccagtggccacgagtgcagatgaagaaacccaggccccaagagggtcatgcaggtagcccagggagttcagccttgaccctgggtcaatgacctttccacagttccacactgctccccttttaaaatccggtgatgtctttatgtcttttgttatgttatcttcaatgtggagggactcgaggtgatctaagcaaactttttctatcttctgcttgcatacctctgagaccaggggactcactcacttgcatgactgggccctgcaggtcacactggccaggcagatgtggtggaggaactggcagaggactttttctagactgtgactacatttagtccacccagcggcccccctatgaagtccagttgagaactaggactctgggggccggtggacagagaagagggagggttctctcccttactgacttccttctgtggccagacattgagcaaggcctctgtacagcatgtcctggggctggccttgccgtagctgctaaatagttgacgaaaccagtccagagaggggaggtgactgccagggtcgcacagctcaagctggggaactcgctgggaaaactgtcagctctgggcagcagcttgacttccactgtaagccccagcccccagggtcaaacactggctctggtgctggcagaggcagcccactagcctgtttcaaaggctgagaaggcccaggagtctgccctgtgctccaccagttctgccctgagactttcctacagagtacaggttttgatgttcagttttaaaggcaagaatcaataaccttctgccccatcaggtgaccccttgtgcctgtcccacccctttattgactgacctcggctcagtcaggtcagttcctgaaggtcagtgtgtggaggggaggctgttctttcccagaaaggccttccccaggcctggtgctctggcctctggaggacttcctggagaagtcccttctttggggtcccagtcagtgtatgggaagcccttattgcatgacctggcacggggcaggggctcaacagtcactattgccttccttgccactgccatttcctcctctgtaagcaggtgattgtgtgtccagtctgagcacagagataagcacacagcaggtgcttaataactagcagctgtaggctgggcgcggtggctcatgcctgtaatcccagcactttgggaggccgaggtgggcagatcacctgaggtcaggagttcgagaccagcctgttcaacatggtgaaaccccgtctctactaaaaatacaaaaattagccaggcatggtggtgggtgtctgtatcccagctacttgggaggctaaggcaggagaatcgcttgaacccaggaggtggaggttgcagtgagctgagatcgtgccactgcaatccagcctgagtgatagagcgagattccatctcaaaaataaataagtaaataactagcagctgtaaatgtggctgttgttcttcacctccacactcagtgccactccactccctccctccgtggtgtgaggggcctcactagctgtctcctaggaggagcatggctgtgagattccagctccatccttggccacggctcctggagacatcttagaggccaggatccagaaggctcccacacctcatttgacaggggagaagctgtcagttccaggtccccttgcacatcagggccagagctgcgttaggcctccagtctccaggccactgggccagagctcacaggctggcagagggttagaactgttactggtggctgggtgcagtggctcacgcctgtaatcttagcactttgggagggcaaggcgggaggatcatgaggtcaggacatcgagaccatccttgctaacacggtgaagccccgtctctactaaaactacaaaaaattagccgggcgtggtggcaggcgcctgtagtcccagctactcaggaggctgaggcaggagaatggcgtgaacccgggaggcggagcttgcagtgagccgagattgcgccactgcactccagcctgggcaatagagcgagactccgtctggaaagaaaaaaaaaaaaaagagctgttactgttgacagtagcatgaggtagaccatggcctgcaccaaaatgggggagtggagtgccactgaggccagaaggaaccacaccctcaagggtggggagttatggtatggggggtcctaggcatggagtcttttaattctttagacaatcctgggagcaactgtccctgtttcacagagggcggggccacacagctggtgagtgggcagccaagactctgttcaagtttgtgtgggtccaacacttgcggccacggtggaggggcatctgagccaggcctcagagagtggcgggggaagttgggtggggaagtgtgcccttctcattcctctgaggctcatcctcttggtgcctctctttcatggaaagggataataaggttattgtgaggatcccctgagttcgtatattcagacgcttagacagagccaggcacagagaagggcccggggttggctagtttgattgctggtgtaattgctaatatcttccagtttgtattggtcaaggttctgcagagaagcagaaccagtaggatgtatatattaagagtttcaagctcatgtgaccgtgcgggctggcaagtctgaaatccgcagggcaggccaggcaggctggcaattcctgcagaatttgatgttgcaatactgagtcctaaggcagtcctggggcagaattccttcttccctgggaggcctcagtctgttctcttaaggccttcaactgattaaatgaggcctgcccaagttatagagagtaacctgccttactccgtcttctgatttaaatgttagtcacatctaaaaaatattttcgcagcagcatttccactggcttttgaccaaacatcaggccacaaagttgatccccaaaattaaccatcactctgtgcctgtaagggaggggctgggaaaggggagcaggtctccccaaggggtgaccttggctttgttcctcccaggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtaggtgctggggctgctgccccaaggcgcgggtagggggcggagggcggagggcggagggagggcgggcgggcaggcgggcttcttgtggcacgtgggcttcttgtggcacgttcctggaggccgaacccttctggctttggaaggagtcgtcagagacccccgccatgcgggaggctggggaggaaggggctcgaaacctccatcatcgcagagtctgaatagcagtggccccgccatgcgcccacgtagcggcgcctacgtagccacgcccccacaccccgtcctggccactctccctcctgaaggtcttctggtacccgccccctccccatctccatccccaggccctgcgtcctctgcccaatactctttgggcctccctgttgtccagctctctccgcggctccatgactgacaacttgagcaaggctaatgtgaatgggagcggttgagggctcagacctctcacccgaggaacatccacagagtgtgccgcatgcccggtgcagtgtggctgcggggacacagacacggagcctcggccctgaggagctggggggcagtgaccgtccctcctctgacccaccactcctccagtgtcaggacactgcgggtatctaggggaaggaatcttgttccacttcaagtctggaacttcaagtctgtgtgtgtgcgtgcgcgcgcgcgcgttgggggtgggggttgcagagcagatgcgtacctgacagcggtaacctaggtcccccctggcctatcaaggcttccctggcggccgaatttaaaggcatcaagcaaacaaagcccaacacatctctgccttgtcctctcagtttccccccgtggcacttagaaccacttgatacaccgaatagtttcctatctcccccactaggatgtaaactccacaggggcattgggaatgctgcctggctatggtagggacagaggggagcaccagggcggggcaggggtgccagagttctgcctgggcagtcagattttccttaggaggggacatttgagtgggacccaaacaggtgtatagcagttgtccagcccagctggcaaggcctgagtctgcctctgcaacccctctcttgggctcctttctctgccacccacctcctcacctttccaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggtaagtcaccaccccactgcctcggccaccgtgatgctaacagcccctttggcagtcagggtctgtgccgggacctccagtgccaggctctgtgcagggggaccagagatgaagtaggcctgatggtgccttcaaggacactcagtctgatgagggaggcgagtgcacagaggaaacacgaggtcagggctgtattagagggagcccagaggaggcacctgcccagcccgagggtcagagaaggcatcttggaggagggacatttgatcgggagcttgatggatgaataggagttcacctggccgataagacagcaactaccaaggcttagaggtgtgagaggaggctgtcttacctcactgagtaaggactgcaggcggcttaccttcgagaagagagcttagtgtctgtgtgcacgtgtgtttgtgtgtatgtgtgtgcgtgtgtgcactggcaggagtcccctgctggggcaggagggccgggccatcaccatctttcaccattcacccctgcaccaggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggtaagcaggatggcagggtgggcaagtccaggctggggcttgggaggtctgtgtgaccttgacagtctctcccttctcccttgtctgtgtaaggaggatgacgccaccttaaataggattaaatgagaatggggctctgaaagggctgtgcaatattttcataacgtgtttttatagagacagttgagtatgttctttaagccctcctctctcctaccatgaactaaagatttctgtggaggtcccctcactcccagcaccccctcctcatcccaggccctttttgcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggtgactgtacccctccttcgtgtgtgtgtgtgtgtgtgtgtgtgtgtgtgtgtgtgtgtgtgtgtgtgtgtcagtgctgggccctcagggacccccagcaagcccctccatcctccagactccagctcttctgtaagcttacagggctggccagaccaggagtggggcactcctcacttcacgcggctgggggctgctggagagagccacagcgggaagggtttcctagaggctgcaggacagtgctggatggattttcaatgctcacctgggtgtgagcgtgcggcagggccgcgtgagggtcagcgatctgctactctggactcagccatctctaggcccctctcactcaggtgctccatggttctgggagctgagaaatctcaaaccagcaaaaaagtggaattgatgttgatgctacaggatagtgcacagatgccatctggttgcagcattttggtggaagggcagtgcccagctaggagagtgaggaggggcaggcatttctggcttgaggagatggggtcttaatgctcgtgtgagaggcagagtgggtggagtggagctggctggatccttgctttggcctcctggatttctctctatctccattttgaaaccactctgtgtttggaagaacttttgagtattcagagctgcccactggcagaacagtcttccttgggcaggagtgagctccttgtccccagaaggctgggtctggctggcccctggcagggacactgatgagggtgcttgagttgatcctgtctagtccctttctgtgttttcaaagcccattctaaagcagattcccatttccgtctttgactctaaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggtaggaggctgggcttgccctggggtgaggaggggtctctttctccttatgcacccactgcccgcgaggcttggtcctcacaagtgtgatccatgagactcaagcctgacttgcagttccatactctggttctgccacttccatgccctttgagcctgggcaggtgaccttacttctcctcatctcagcttcctcctccataagagggaaaaaggtattacctgcctcattgtgttgcaaggagatgggcagcatctagggcactggcctggagtatcgcaggtgctttgcctaaggtggtgcagtccaggagaggcagctccagagagaggcccccggctggggctgaaaggagggcagacctcggtttgaatttcaccctgccgctctatagctgtgtgacttgggcaaattacttaacatctctgtatgaggaaatgatgagtgctaagcacttagcttagtgccgggacaatataaattctagctatcgttactattgttttcatcacccgttgctttaaaatccagcctctggtataggcaactattgacgggctaccctgtgtcgaaaacatgcccaggcaggtagcaggaagtcacagatggggacctcttggggcatcaagggatggtgccctgaggctgagctgttctggttgggtggagcatgagaggtctgggaagacagtgggactccagcctggaataagaggctcagagttgattctcgtctgagcacgtccaggggaaccactgagggtttgggaacaggagagtgagggtgagaacctggttctgggcacagcaggctggcatgtaggatggatgttcaggaaagatgagcatagtcaggtggctggtgcccttgtccaggggagaggctccgtcaggttcaggggtcctggcttggagggaagtccgccatgctctaatcacgctcccctttggaagtgctcagccgatgagctcacaggcacatgtcagtttgaagtcatggaatctgactccatgaagcgcacctcaaagagcaccattttgcagctaagggaactgcaggctggacatgctgagtggctgccccgagcccttgcagctaggacatagagaatgctagtaaccacaaccctaccatgttcagagcacatgccaggctccatgctggggcttcgcacgtgtcatcttcacagtgtccctgtgagtaggtgtggtttctctttccatcttacaaatgagtaaacagagcctcagtgtagctaagtaaccactattttaggtttcttagccaatgggtgtgtctgactcctaagcccatggagggcattctgaggtggttcagacagaccccggcttacccttgaacttctgcctgctggctgcatagggaggggctggggggagtttgagcatctcaggccatagagcccctgcctcactgtctccatctctgggtggaaagatggtgttttccctgagaaactaaggctcagagaggttgaatggctctcccaaggtcacacagctggtcagctgcagagttgagaacacaggagtcctggtgctcaggccagcatctctttttttctttgagttgtttctaggtttcctagctcttgcctcagaccttaaagagagagggtctgatggggatgggcactggagacggagcatcccagcatttcacatctgagctggctttcctctgccccaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaagaggcccgtgaggccgggtgggtggggtgctgcgtgtctctcctgcacagcttttctgtgtcagtttgtgccaccaccataccgccatgcatcagggtggcggtttgccaggtagatgctgtgggcagcttccgccattgtgtggacagcatgtatatgtgtctctgtgtggctgggtctgtttttgcttttgtccagatcagtaaggtttgctacctgggtaccccactccacttggagtagaatgtgcataaatatggcataaagaaatgcaatatgcatgcatttattgattgatctatttttttctgagatggggtcttgctgtgttgcccaggctggtctcaaattcctgggctcaagcaatcctctggtctcagcctccccaagtgttgggattataggcatgagccgctgcacctggcctctctgatctatttaacaaacctgctgggagggtctcagggtcaggagcagcactgggctctgaggacacagagctcactcagccgtgacccagagggggtgcctgagctgcatgctgaaggttgttagcatgaccagcaaggcaagaaaaggccctgccgagattagcaaggcatgtgccaagccctggaatgtgacagccgggccttctagaaacctgagtgtataactctccttaaaagccagtaggagctcctcaaaaggcagccctaaggagtccactcttaaatgaactcagagtcagttttaaaatgcaagtctgtgttgattctggtctggatggtgcattcctcgagagcaaaagacagtcttggtcttggatccacttgccctgggtacactgagggctgctaggttccaggtgctcttcctggcactggggagggatacaggcccaagagacatgctgttctccctcctggagcatctattttagtggaggaagacagaaaacaaaccattaatatagagtactgaaaagatgcgatggagaaaactatagcaaggaagggaatggggtgggagagaggtcaggagaggtctcgctgacaaggtggacgaaacaggccatgaggcagagaacatgttccaggcaaagcaaaggcccccaggtggggatgtgcagggagtaccaggaaaccagagaggtgggaatagttatgagatggggggtgcctcagaggggacagggccaagtcaggtgagacctgagggtcacagtcagcagtgagctggggccatgcaggggtctggcctcagaggagtgtggtctggcctggatctgaacctctcactgtggcctagctgctgagctgagaagagatgacaaggaccttgggcagaagcagggagactggagggaggcggtggagggtccaggcgttggggcggggctcaggctggagtctgaagggagcctgcaggcctggtgggtggatgtgggtgggagagggggaggatggcaccaaggctcgggcccctggacagatggagttgccattaagtgggatggggcaggctatggggccatcagtttcagagggatgagtttggcactggcatggtaggcatctgtctatctccacggccctcaaaccaggcatgaagcaggagctcacgtgtttggtcagccatggtgcagaaccgcctgggtgggaggtgcggggtgggagatacacggttgtgtcccaaatgggctctgagccagcgagggccgtctgcactttggcctcacagaaggatgtcggagggagaaatgaagtgtgggtgggggtcccgggccacgctagacatgtgctttcttttcctcgggctctggcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa

SEQ ID NO: 3 - DNA, 42802 bp, Homo sapiens
  source 1..42802: mol_type = genomic DNA
ctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcaggtaaggcttgctccaggcgccagaataggttgagagggagcccccggggggcccttgggaatttatttttttgggtacaaataatcactccatccctgggagacttgtggggtaatggcacggggtccttcccaaacggctggagggggcgctggaggggggcgctgaggggagcgcgagggtcgggaggagtctgagggatttaagggaaacggggcaccgctgtcccccaagtctccacagggtgagggaccgcatcttctttgagacggagtctagctctgtcgcccaggatggagtgcagtggcacgatctcagctcactgcaacctccgcctcccgggtttaagcgagtctcctctctcagcctcccgaatagctgggattacaggcgcccaaccaccacgcccgcctaatttttgtatttttagtagagacgggttttcaccattttggccaggctggtctcgaaccccgacctcaggtgatctgcccaaaagtgctgggattacaggcgtcagccaccgcgcccggccgggaccctctcttctaactcggagctgggtgtggggacctccagtcctaaaacaagggatcactcccacccccgccttaagtccttctgggggcgagggcgactggagacccggatgtccagcctggaggtcaccgcgggctcaggggtcccgatccgctttgcgcgaccccagggcgccactgccatcctgagttgggtgcagtcccgggattccgccgcgtgctccgggacgggggccaccccctcccgcccctgcccccgcccctttggcccgccccccgaattccattgggtgtagtccaacaggccaccctcgagccactccccttgtccaatgtgaggcggtggaggcggaggcgggcgtcgggaggacggggcttgtgtacgagcggggcggggctggcgcggaagtctgagcctcaccttgtccggggcgaggcggatgcaggggaggcctggcgttcctccgcggttcctgtcacaaaggcgacgacaagtcccgggtccccggagccgcctccgcgacatacacgagtcgccctccgttatcctgggccctcctggcgaagtccccggtttccgctgtgctctgtggcgacacctccgtccccaccttgtcctggggggcgccctcgccccaccagccccgatcaagttcacagaggggcccccggccaccctcaaggcctcggttccttacgaggttgaaacgttgcctcagaatctccccgcccctccttggtctgcagccgagatcttcagccacggtggggcagctatcccccgggaccgaccccctggggtggcctcgcttcttcagaggctgtgaatggcttcggttcagctgtccaagcggcgatttttcctctgggtgaaatggattagattttagatttccacaagaggctggttagtgcatgatcctgagttagagctttttaggtggctttaaattagttgcagagagacagcctcgccctagacaacagctacatggccctttccctcctgagaaccagcctagcctagaaaaggattgggattgcctgatgaacacaaggattgcaggaaactttttttttaattggcaagggggttggctttgactggatggagagctttgaactgccttgaaattcacgctgtaactaacacaccagtttcctctgggaggccagagagggagggagggtgtaatgaaatacggatgattgttcttttatttttatttacttatttattttttaactttttgtagagatgaggtctcgcttggttgctcaggctggtcttgaactcctggcctcaagcgatcctcctacctcagcctcccaaagtgttgggattacaggagtgagccaccgcgccccaccggggatgatgatgattgcaaacattctgccactcagttttacaaaagaaagagaggcactggattaatgtgtatctcactcaccaatcaacctcttccttaagagaaaatgttaaggaagtcttaggcaaggccttgtttgttcatcactttagtttctctctcccgggatggctgagaatgtgatgtttcctctgttgtcaaggagactacacccctgatgttttcctccagacttctgagagctggtgtgtgtttctagcactttctagctgcaccacctcacgctgtagctggcttcaaggcatatccaggggggagtttcttgtccatttcctttacaaagggaagttgttggaatctgaaccgcaagccttcacttagaccaaaatcaggcaacagcggtgagcgcagctccaaacgtgtcaatgactcacccaaatttgagtaagggagttggctgctttaacgagccgcagggtgattcccttgtcatttccggaaatacctatcttccagggaacactgggaaaaaacagggagacctttgttgagacagaaaacctgtaggggaattctgttcctcattcctgctcttatctgtagacttcctccctgataagatccaattctagatgggtcggttgctccttgctttgatgggtgctttgatgggctttattattattattattattattattattattttgatgggctttttgatgtcccttttccttccacactctgtcccaactgtcaagcaaatagccttttgttgctaagagactgcagatgtaaccgaccagcagcaaacagtgagtcaggctctctcttccggaagcaaaatcaattgctgagatcactctggggaaaatacccaccttatttggaaagaagcactgatcaattgatgtctattttttttttttttgagttggagtctcgccctgtcacccaggctggagtgcaatggcataatctcgcctcactgcaatccccgcctcccgggttccagcaattctcctgcctcagcctcctgagtagctggaattataggcgcctgccacaacacccggctaatttttgtatttgtagtagagatggggtttcaccacgttggccaggctggtctcgaactcctgacctcgtgatccacccgcctcagcctcccaaagtccaaggattgcaggcgtgacccactgtgccagccaatcaattgatttctcattcattttcagctggctctgttcccttaagccaggggattttcgtttgtttgtttccccttcaaggaaatgattctagctacagttttgatttccttgtacaactgttttcagtagcacagggaaagaaaacatcgaaagcattcaccacctcatttgtgtgctgggggaaaaagcagaaatgtgtattctctttttttgtttcgatgaccttgttcctgacttgttactcgtgacttgagagatcagagggctagaggactagaatttatagaggtgttttttttgtttgtttatttttgttcgagttgcccaggctggagtgcagtggcgcaatctcggctcactgcaacctctgcctcccaggttcaagcgattcttcggcctcagcctcctgagtagctggaactacaggcgcccgccaccacacccagctaatttttgtatttttcagtagagatgggatttcaccatattggtcaagctggcctcgaactcctgacctcgtgatccacccgcctcagtttcccaaagtgctgggagtacaggcgtgagccgccgtgcccggcctttttgtgtttttgtgtttttgagaggagctcattgctttttaggcttccctagcgtgagaaaatctggggatccatgctctagtttacttcctttttttttttttttttgagatggagtctcgcttagattgcctaatctcagctcattgcaacttctgcctccggggttcaagggattctcgtgtctcagcctcctgggtagctaggatacgggcacccgctaccatgcctggctaattttgtacttttagtagagacagggtttcgccacgttggccaggctggtctcgaactcctgacctcaggtgagccgcctgccttggcctcccaaagtgctgagattacaggcgtgagccaccgcgcttggcctaatttgcttttcctgaaattcaaatggtctaatatgaaaaacgccaaccttgcttgaaagaataagaaagaggtgcggtttcgttgggccgttgatgtttggaacaggactggttttgtccccttgctcggaaagggcagcaactgtgaggacagctccctgacgtgctctcactcagcactgttccgttcctgagcactgtccccactagctaggccaagggagctcatttggcaggcaactgctgtctggctgcgcctgtggcagtaaaatctgcctttattttttggaggcagggtcttgccctgtcgctcaggctgaagtgtgcagttatagctcactgcagcctccagcttctgtactcaactgatcctcctctctcagcctcctgagtagctgggactatacgcacgtgttaccactcccacctcagtttgtttgtttatttatttatttatttatttattgagatggagttttgctcttgctgcccaggctggagtgcaatggcgcgatctcggctcaccgcaacctccacctcctggttcaagcgattctcctgcctcagcctcctgagtagctgggattacaggcatgcaccaccacgcccggctaattttgtatttttcgtagagatggggtttctccacattggttcaggctgttctcgaactcccaacctcaggtgatccacccgcctcagcctcccaaagtgctgggattataggcgtgagcccccgaacccggccactcccagctaagtttaaattttttgtttgtttgttcgtttgtttttattttttgagacagagtctcccgcccaggctggagcgcagatcactgcatccttgacctcccaggcttaagccatcctccccactcagcctcccaagtagctgggattacaggtgtgtgccactatgcttggctaagttgtgtattttttgtagagatggggttcaagggattctcgctttgttgcctcggttggtctcaaactcctgggctcaagcagtcctccctcctcagcctcccaaggtgctggggaaatccacttttgaaacattgtctggagagttgcccaggtggtagatcacagaaataggtcatcgtggggtccttcccatgggtgcagtcttgagccacctgtggccagcaaatatttggagaataatagtcaggggagagcttgaggtccagggaaaggttttgtttttcttcagggaaaggtttttattgttctttatccctccttaaaggaccttcaggtgttactgacattcccggtctacccagtggcacatttagtttgtaagctgggccctcgtacagaggtagggaggtgagagcattggattagtggtcaccaaagctgcggtcacctagtggggtgatcagaggctcctcccttaagatcttgattgccaacgcctctggcccaactttcctttttatttatcgcaagcctcctggaatctcaattgctttttgcccacccggtgtgtcagcacaagaaatgagtcatttcctcctttaagcacagttgaaattgagctgtgagtcagtgaggtgtgtacgatattgtcaaagcggggtgtgtacagtattgacagatctgtagttgggcaagagaattatcagagtttgtgaccacagcagattccaaagctcgactcattttcttctctcttccttcccttttttcttttctttttttttttttttttgacagagtctcgctctgttgcccaggctggagtgcagtggcacaatctgggctcactgcagcccctgcctcctgggttcaaatgattctcatgtttcagcctcccgagtagctgcaattacaggcattcgggttcaagtgattctcctgcctcagccacctgagcagctgggattacaggcgcccgccaccacgcccggctaatttttgtatttttagtagagacggggtttcaccatgttggccaggctggtctcgaactcctgaactcaggtgatccgcccacttcggcctcccaaagtgctgagattacagacgtgagtcaccgcgcccagcctgttctgttctttaattctcaaaacaccctctaggaagtagagactgccattctcccccattttacagatcaggaaactgagtcccagaaggatttagtcagttacccaagttgttctagttaaatggcctggaaagccagtgaagcccaggattgtctatctaacccccttactactctaactttcagggaatccacatgaatgtgctgggtcaaccatcaaagttgaaatggataaagggggctggatgcggtggctgatgcctgtaatcctagcactttgggaggccgagatgggtgggtggattgcttgagcccaagagtttgagaccagcctgggcaacatagtgagacacctgtctctgcaaaaaataaataaaaagttagctgagtgtgatggtgcacccctctagtcacagctgttgagttaggcttaggcaggaggatcgcatgaacctgggaggtggaggcggccgtgagcctcagtcatgccactgcactccaacctgggcaacagagtgaaagccggtgtccgaaagagaaagaaaaaaagacatagatacatcttttaaagttaggttgtatgttaattacctacaactcagtttcaactgtgcttaaaggaggaaatgactcatttcttgctacatatcaaattagcccaaaatgtagtggcttaaaacaacacatttatgatttctcagtttttgcgtgtcaggaatttggaagcagcacagctagacggttccagctcagggtctctcatgaagttgcaatcaaaatattggcaggagagaaaaacatattttcagaagctgcaggcataggaagacttggctggggttgaaggatccacttccaagatggcgcactcagtggctcttggctggaggcctcagttccctgctgcgtggagctctccctccagctgcttgagtggactcatgacatgcagctggcctcccctggagcagtcgatccaacaatgagcatggccatgaactaggctcagaagccactccctgtcgtctctacattttcctatcagaagcaagtcattaaaagtccagtgccactccaggggagacgaattaggctctgccttctgaaaggattatcacagaagatgcggtcctatattctttttttaaaattattcttttttttattttgtagagatggggtcttggtatgttgcctaggccagtctggaattcctgggctcaaacaatcctgtctctgcctcccaaagtgttgggattacaggcatgagccactgcacctggtcatgtggtcatattttctttttcttttttttttttttttgagacagagtctctgtcgcccaggctggagtatggtggcgtgatctcagttcactgcagcctccgcctcccgggttcaagcgattctcctgcctcagcctcctgagtagctgggattacaggcgcccgccaacatgcccagctaatttttttagtagagatggggtttcaccatgttagccaggatggtctcgatctcctgatttggtgatccgcccaccttggcctcccaaagtttcaaccatcgatcagaacttattgatgtacttatgtagctaggcacggtggcgcgtgcctgtaatcccagctacttggaagggttaaggcaggagaatcgcttgaacctgggaggcagaggttacagtgagtcaagatcataccattgcactccagtctgggcaacagaatgagactctgtctcaaaaacaaaaaacaaacccttgtatgtgattttcctggatagcatctgttacatcttcacaaagataaaaagtcagacttggctgggcatggtggctcacacctgtaatcccagcactgagaggctgaggcaggcagatcacttgaggtcaggaatttgagaccaggctgggcagcatggtgaaaccccgtctctacaaaaaatacaaaaattagccgggtgtggtgtcacgcacctgtattcccaagctactcaggaagctaaggcaggagaatcacttgaacccagaggtggaggtttgcagtgagttgagattgtgccattgcactccagcctgggcgacagagtgagactctgtgtcaaaaataaaataaaataaaattttaaaaaaggcagatttttttttcttcttggtattgttaccttattatagtaataataagtgcatagtgcatgctgagataagcaatcataatttgttattgcggccgggcatggtggctccagcctataatcccagcactttggtcaggagttcaaggccagcctggccaatatagtgaaactccatctctactaaaatacaagaaattacctgggcatggtggcagttgctggtgatccccagctacttgggaggctgaggcaggagaatcgcttgaacctgggaagcagaggttgcagtgagccaagattgcaccactgcactccagcctgggtgacagagtgagactctgtctgaaaataataataataataatttgttattgcttttattgccttagtttacatagggaatcaaagtttatactttgatttataaaagttgctttgattctagttcacagaaccagaatctttcatataaaggtattagagggcccagtgtggtggctcatgcctgtaatcccagcatattgggaggctgaggagggaggatcactttaggagtttgaggccagcctaggcaacatagtgagaccttgtctctacaaaaaattccaacattagctgggcatggtggcatgtgcctgtagtcccatttatttggggggctgaggcaggaggatcacttgagcccacgaggttcaatccaggttgcagtaagccatgatcctgccactgcactccagtttgggtaacagagcgaagctatgtctcaaaaaaagaaaaaaaaagtattctaaatccaaatttaatatataaaactaaatgcaggccaagtgtggtggcatatacctataatcacaacactttgggaggctgaggtgggaggattgcttgagcccaagagttcaagaccagcctaggtaacacagtaagaccccatctctacaaaaagtagaaaaattagcctggcatggtggtgagtgcttttaatcccaactacttagggggctgagatgggaagattgcttgagcctcagagtttgaggctgcagtgggccgtgatcgctccactgatcgctctaaagtgagaccctgtctcaaaaaaaaagaaaatagaagaaaactaaatacattcaataagactttgatctcttttccaaggtgtaaatatattttgggaaattttccagttactttgttctcattttaatgtaataatctaagtcttggttttctaaggaaaagttttctcttattatatcttttgttaatgtttctctcccatttcttttgatctgatcttcagatacatgattatcttcactgctaaatttgtgttctctggcctctacatttataatttctcataattctttatctaagtatttcttccctacctactgaagaaaactcaagttttcttccaccttaatgattatgctgtgtctgtgagttttcttcatgactctttacagtacaagttttttgtttttgtttttttaatggtcagatggatagaacaacacaggttttgtttgttttgttttaacttttaaaaaaattataatagataaagggtctcactacgttgtccaggctgatctcatactcctgggctcaagcaatccacccacctctgcctcccaaagtgctgggattacagtcatgagccaacatgcctgggcagtacaggttttttttgagacggagttttgttcttgttgccgaggctggagtgcaatggcacaatcttggctcaccacaaagtctgcctcccaggttcaagtgattctcctgcctcagcctcctgagtagctgggattacaggcatgtgccaccacgcccagctaattttgtatttttagtagagacggggtttcaccatgttggccaggctggtttcgaactgctgacctcaggtgatctgcccacctcggcctcccaaagtgctgggattacaggcatgagccaccatgcccagctgtagtacaggttttaatatgctaaatactcttcctttctttattaatgtgcatggaagttctaatatttttttcccataccccagagagtccatattttggaatcaacaacactagcctttgttgacaagtgtctctcttgggttccttctttgtgtcctccactgaattttggggttcataaaatttcatttgttgtgcttgcttaattccctgggaatcagactgttcctgatcggatgacatttctggttaattctttagttggcaggaaatagacacaggaaacgtggtcagtttctgattctggcgttgagagaccctttctccttttcctctctctcagtgggcgacagatgcgaaagaaacgagttccagtgccaagacgggaaatgcatctcctacaagtgggtctgcgatggcagcgctgagtgccaggatggctctgatgagtcccaggagacgtgctgtgagtcccctttgggcatgatatgcatttatttttgtaatagagacagggtctcgccatgttggccaggctggtcttgaatttctggtctcaagtgatccgctggcctcggcctcccaaagtgctgggattacaggcaccacgcctggcctgtgacacgattcttaacccctttttgatgatggcggctggaaaagtggccagtggattttgatgtattcaatcatgaattaggaggtggggagagaatgaattattggagctttccttaaagccattaaatggctctattgttttttcaattgatgtgaatttcacataacatgaaattaaccagctcagtggcattaatacatctgcaatgctgtgtggccaccacctctatcttgttccaaaactttgcataacctaatgtctttttttttttttttttttgagacggagtctcgttccatcacccaggctggagtgcagtggtgtgatctcagctcactgcaacctccgcctcccaggttcacgccatcctcctgcctcagcctcccgagtagctgggactacaggcaccctccaccacatccggctaattttttgtatctttagtagagatggggtttcaccatgttagccgggatggtctcgatctcctgacctcgtgatccacctgcctccgcctcccaaagtgctggcattacaggcgtgagccaccatgcccggcctatttttttttttaagagatggagtctaattctgttgcccaggctggagtccagtggtaccatcatacttcactgcagccttgacctcttgggctcaagtgattctcttgcctcgaactcccaaagtattgggattacaggtgtgagccaccgcactcagcctaatgtccagtttttaacaagctccatttaaatgccctccgttttgacccataaaggggtaggcttggccgggcacaatggcttgtgtctgtagtcccagctacttgggaggctgaggcagaaaggcagaaagattgctttataaagcccaggagtttgagggccacctgggtggcatagctagacctcatctctaaaaaataagtaataaataaatatttgtttttgtttttttctttttcttttcttttttttttttttttgagacggagtcttgctctgttgcccaggctggagtgcagtggcgcgatctcagctcactgcaagctgtgcctcctgggttcatgccattctcctgcctcagcctcccgagtagctgggactacaggcgcccactaccacgcccagctaattttttgtatttttagtagagatggggtttcaccacgttagccaggatggtctcaatctcctgacctcgtgatccgccagctttggcctcccaaagtgttgggattacaggcgtgagccactgagcccgccccatatgtatgtatatatatatttttttaaaatgggagaccaggcatggtggctcatgcctagaatcccagcactttgggaagctgaggtaggcggatcacttgaggccatgagtttgagaccagcctgctcaacatgatgaaacttctatctctactaaaaaaaaaagtgggattaggtcaggcacggtggctcacacctgtaatcccagcactttcagaggccgaggcaggaggatcatgaggtcaggagatcgagaccatcctggctaacacggtgaaaccccgtctctactaaaaaaatacaaaaaattagccaggcgtggtggcgggtgcctgtagtcccagctactcaggaggctgaggcaggagaatggcgtgaacccgggaggcggagcttgcagtgagccaagatcgtgccactgtactccagcctgggcgacagagcaagactctgtctcaaaaaaaaaaaaaaaagtgggattgacattctcttcaaagttctggggttttcctttgcaaagacaggattggcaaggccagtgggtcttttttgtgtgtgtgtgtgtgacggagtctcactctgccacccaggctggagtgcaatggcaggatctcggctcaccgcaacctcctcctcccaggttaaagtgattctcctgcctcagcctcccgagtagctgggactacaggtgcccgccaccacacccaactaatttttgtatttttagtagagacagggtttcactatattggccaggctggtcttgaacccctgacctcacgtgatccacccgccttggcctcccaaagtgctgggattacaggcgtgagccactgtgctcggcctcagtgggtctttcctttgagtgacagttcaatcctgtctcttctgtagtgtctgtcacctgcaaatccggggacttcagctgtgggggccgtgtcaaccgctgcattcctcagttctggaggtgcgatggccaagtggactgcgacaacggctcagacgagcaaggctgtcgtaagtgtggccctgcctttgctattgagcctatctgagtcctggggagtggtctgactttgtctctacggggtcctgctcgagctgcaaggcagctgccccgaactgggctccatctcttgggggctcataccaagcctcttccgcccttcaaatccccccttgaccaggaggcattacaaagtggggatggtgctacctcttcgggtttgtcacgcacagtcagggaggctgtccctgccgagggctagccacctggcacacacactggcaagccgctgtgattcccgctggtcgtgatccccgtgatcctgtgatccccgccccgtgaggctgaacacatagtgacgcttgctagccaagcctcaatgacccacgtaacatgaagggggaaaagccagaaagttctgccaaggagcaaggccaagaatcccgaagggaaatggactttgaagctgggcgtcttcttggctgtcttaatacaagtggcacatccaaatccaaaaccccgaaattcaaagtcttgagcacccgaaattctgaaacgtcttgagcactgacctttagaaggaaatgcttattggagcattttggatttcggatttttaccactgagtgtggagtcctaattaggaaaaaaaccaggctgaccgaaccaaaggaaagcaataaaagaaggcagatagggtcaggcacggtggctcacccctgtaatcccagccttttgagaggctgaggcgggtggatcacttgaggtcaggagttcgagagcagcctggccaacacggtgaaaccccatctctactgaaaatacaaaaactagccaggtatggtggcgtctgcctgtaatcccagctactcgggaggctgagacaggagaatcacttgaacctgggaggcagaggttgcagtgagccaatatcacgccattgcactccagcctgggggacaagagcgaaattctgtctcaaaaaaaaagaagaagaaggccgacaaactatgtaactctgcctttctccatggtccagaacacacagccctcctgcgtaaataactccttatcttcctgctcccagctatcatcagacacctcggctgatagaaaattgcaagttagctcactgcaacctcggcattataagtactgcacaaagccctcttcagcgcacagcacaagcaccattctataaaatctccagcaagcggccaggtgcagtggctcatacctgtaatcccagcattttgggagactgaggcgggcggatcacctgaggtcaggagtttgagaccagcctggccaacatggtgaaaccccgtctctattaaaaatacaaaaaaattagccaggcgtggtggcaggtgcctgtaatcccagctacttggaaggctgaggcaggagaatcgcttgaacccgggaggtggaagttgcagtgagccgagatcttgccatcgcactccagcctgggggacaagagtgagacttcgtctcaaaaaaaaaaaaaaaaattcccagcaagcctttgtcttctggcagtcagctcctctcttgctgacctgctcattgctttcttgcaaggtattttcctacctactttctggaataaatctgtctttctgtacttacaactaccttttttaaaatttctttcttttttgagatggagtctcactctgtttgcccaggctggagttcagtggtgcaatctcagctcactgcaacctctacctactgggttcaagcgattctcctgcctcagcttcccgagtagctgggattacaggcgtgcaccagcacgcaggctaatttttgtatttttagtagagacggggtttcaccatgttggccaaggtggtcttgaactcctgacctcaagtgatcctcccacctcagcctcccaaagcgctaggattacggccatgagccactgaggccggctgcacctacaactgtcttgataaattcttacccccacaccactggtccagatagtcagtgctcacccacaacattaaggatattccaaatttgaaacattccaaaatcagaaaaatattccaactctgaaaatattccaaaatccaaaaaaattcaaaatccaaaacacttctggtcccaagcattttagagaagggatactcaacccaaaataaggacagcaattctataaattgtgctaccatcttgcaggtctcagtttaacagctttacacctattagcgcaccagtgctcatagcagtgctgggaaatgtgtacagatgaggaaactgaggcaccgagagggcagtggttcagagtccatggcccctgactgctccccagcccgcctttccaggggcctggcctcactgcggcagcgtccccggctatagaatgggctggtgttgggagacttcacacggtgatggtggtctcggcccatccatccctgcagcccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcggtatgggcggggccagggtgggggcggggcgtcctatcacctgtccctgggctcccccaggtgtgggacatgcagtgatttaggtgccgaagtggatttccaacaacatgccaagaaagtattcccatttcatgtttgtttcttttttttcttttctttctttattttgtttttgagatggagtctcactctgtgatttttttcatctctaaatttcctacatccatatggccaccatgaggccccaggctggccgatggttgctgttagcttattgggaaatcactgtttggaaggtgctggttgttttttgttgtttgttgtttttgtttttgtttttgttttgagacggagtctcgctctgtcgccagggtggagtgcagtggcgcgatcagctcactgcaacctccgcttcctgggttcaagccattctcctgcctcagcctcccaagtagcgcggattacaggcatgtgccaccacctccggctatttttttttctatttagtagagatggggtttcaccatgttagtcaggctggtcatgaactcttgacctcaggtgatccacccgcctcggcctcccaaagtgctgggattacaggcgtgcactgctgcacccagcctttttttgtttttttgagacagggtcttgctgtcacccaggttgaagtaaggtggcacgattatggctcactgcggccttgatctccttggctcaagcgatcctctcacttcagcctctcaagcagttggaaccacaggctgtaccaccaagcctggccaatttttttgtacagacacaggctggtcttgaactcctgggctcaagcaatcctcctgccttggcctcccaaagtgctgggattccaggcatgagccgctgcacccggcaaaaggccctgcttctttttctctggttgtctcttcttgagaaaatcaacacactctgtcctgttttccagctgtggccacctgtcgccctgacgaattccagtgctctgatggaaactgcatccatggcagccggcagtgtgaccgggaatatgactgcaaggacatgagcgatgaagttggctgcgttaatggtgagcgctggccatctggttttccatcccccattctctgtgccttgctgcttgcaaatgatttgtgaagccagagggcgcttccctggtcagctctgcaccagctgtgcgtctgtgggcaagtgacttgacttctcagagcctcacttccttttgttttgagacggagtctcgctctgacacccaggctggagtgctgtggcacaatcacagctcacggcagcctctgcctctgatgtccagtgattctcctgcctcagcctcccgagtagctgagattaaaggcgtataccaccacgcccggctaattttttgtatttttattagagacagggtttctccatgttggccaggctggtcttgaactcctggtctcaggtgatccacccgcctcggcctcccaaagtgctaggattacaggtgtgagccactgcgccaggcctaatttttttgtatttttagtagagatgcggttttgccatattgcccaggctggtctcgaactcctgggctcaagcgatctgcctgccttggcctcccaaagtgctgggattacaggcacaaaccaccgtgcccgacgcgttttcttaatgaatccatttgcatgcgttcttatgtgaataaactattatatgaatgagtgccaagcaaactgaggctcagacacacctgaccttcctccttcctctctctggctctcacagtgacactctgcgagggacccaacaagttcaagtgtcacagcggcgaatgcatcaccctggacaaagtctgcaacatggctagagactgccgggactggtcagatgaacccatcaaagagtgcggtgagtctcggtgcaggcggcttgcagagtttgtggggagccaggaaagggactgagacatgagtgctgtagggttttgggaactccactctgcccaccctgtgcaaagggctccttttttcattttgagacagtctcgcacggtcgcccaggctggagcgcaatggcgcgatctcggctcactgcaacctctgcctcccaggttcaagtgattctcctgcctcagcctcctgagtagctgggattacaggcgcccaccaccaagcccgggtaattttttgtatgtttagtagagatggggtttcactatgttggccaggctggtgttgaactcctgacctcatgatccgcccacctcggcctcccaaagtgctgggattacaggcgtgacccaccccatgaaaaaaaattaaaaaatgaagcgatgctgggcgcggtggatcacgcctgtaatcccagcactttgggaagctgaggcaggcagatcacgagggcaggagattgagaccatcctggctaatacggtgaaaccccatctctactaaaactacaaaaaattagccgggtgtggtggcaggcacctgtgatcccagctactcaggaggctgaggcaggagaatcgcttgaacccaggaggtggaggttgcagtgagccgggatcacaccattgcactccagcctgggtgacagagtgagactctgtctcaaaaaaaaaaaaaaaaaaaaaagcgaattctgaaatacatgaattcttttccttagatgcctgcttctgtcttgaggtttgttgttgttatttcgaaacagagtcttgctctgtcgctcaggctggagtgcagtggcatgatcttggctcaccacaacctccggctcccaggttcaagcgattcttctgcctcagcctcctgagtagctgggattacagctgaatgccaccttgctgggctaatttttgtatttttagtagagatggggtttcaccatgttggccaggctggcctcgaactcctgacctcgagtgatctgcccgcctcctgaagtgctgggattacaggcgtgagccacctcgtcctggtgagggtttttttttttccccaaccctctgtggtggatactgaaagaccatattaggataactgtacagtatagagaaggcagtggcaagttttctctgtcatataccagagtgggcttgggcatggtggcatactcctgtagtctcagctaatcaggaggctgaggaaggaggatcgcttgggcccaggagttggagactgtagtgagctgtgatcacaccaccacacttcaatctgggcaacagagcaagagaccctatctctaaaaaaaagtaagtatttcggacactgtgggccatacggtctctggtgcagtttctcaacatggctgttgggtgaacacaaccacgcacagaacgcaaaccaatacacgtggctgtgggcccagaaaatgttatttatggacacaaaaattggaatttcatataactgttttgtgtcatgaaaatgatttccctttttatttttatttttcttctcaagtatttaaatatgtaaaagccatttttaggcctggcaggatggttcacagctgtaatcccagcactttgggaggtcgaggcgggaggatcacgaggtcaggagatcgagaccatcctggccaacacagtgaaaccccgtctctactaaaaatacaaaaaattaaccaggcttggtggcgcgcgtctgtagtcccagctgctcaggaggctgaggcaggagaatcgcttgaatgcaggaggcggaggttgtagtgagccgaggttgcaccactgcactccagcctgagcgacagagtgagagtccgcctcaaacaaaaaaatgtttgcccatgctggtcttgaactcctgggctcaagctatctgcctgccttggtctcccaaagttctgggattacaggcatgagctacagcgcccggacttttgttgttttatatctatatatctatatataacttgttttatgtatatatataacttgttttatatatatacataaactgcagtaaaaaacatgtaacataaaatttaccttctcaaaccttattaagtgcacagttctgtgccattagcaaattcacactgttgtacaacatcacaaccaccatctccagaacttttttttttttttttattctttttgagacagagtctcactcgtcgcacgggctggagtgcagtggtgcgatctcggttcactgcaacctccacctaccaggttcaagcaattctcctgcctcagccccctcagtagctgggattacaggtgcccgtcctaccacgcccagctaatttttgtattttcagtagagactgactgggtttcaccatgttggccaggctggtctcgaactcctgacctcaagtgatcctcccacctcagcctcccaaagtgctgggaatacaggcatgagccactgcgcccggccccagaactcttttatcttcccaaactgaagctctgtccccatgaaacactcactctccatcccctccccaactcctggcacccaccattctactttctgtccctatgaatgtgatggctctagggacctcctctgagtggaatcagacagcattttccttttttgactggcttatttcactgagccaagtgcggtggcacacgcctgtaatcccaaaactttgggagaccgaggcgggcgcatcacctgaggtcaggagttcgagaccagcccggccaacatggtgaaaccccatctctagtaaaaatacaaaaaattagcctgtcatggtcgtgggtgcctgtaatcccagctaagtgggaggctgaggcaggagaatcgcttgtacccaggaggcggaggtcgcagtgagccgagatcgtgccattacactccagcctgggcaacaagagtgaaactccgtctctcctaaaaatacaaaaaaattagctgggcatggtggcacatgcctgtagtcccagctacttgggaggctgaggcaggagaatcacttgaacccgggaggtggaggttgtaatgagccaaggttggcggcgaagggatgggtaggggcccgagagtgaccagtctgcatcccctggccctgcgcagggaccaacgaatgcttggacaacaacggcggctgttcccacgtctgcaatgaccttaagatcggctacgagtgcctgtgccccgacggcttccagctggtggcccagcgaagatgcgaaggtgattcccgggtgggactgagccctgggccccctctgcgcttcctgacatggcaaccaaacccctcatgcctcagtttccccatctgttaagtgtgcttgaaagcagttaggagggtttcatgagattccacctgcatggaaaactatcattggctggccagagtttcttgcctctggggattagtaattaagaaatttcaggccgggtgcgtaatccctgtaatcccaacaccttgggacgccgaggcgggcagatcacctgaggtcgggagttccagaccagcctgaccaacatggagaaaccccgtctctactaaaaatacaaaattagccgggcttggtggtgcatgcctataatcccagctactcaggaggctgaggcaggagaatcacttgaacctgggaggtggaggttgtggtgagccaagatcgtgccattgcactccagcctgggcaacaagagtgaaactccatccaaaaaaaaaagaaaagaaaagaaaaaaaagaaaagaaatttcagctgacacagcttcacactcttggttgggttcccgtggtgaatgatgaggtcaggtgatgactggggatgacacctggctgtttccttgattacatctcccgagaggctgggctgtctcctggctgccttcgaaggtgtgggttttggcctgggccccatcgctccgtctctagccattggggaagagcctccccaccaagcctctttctctctcttccagatatcgatgagtgtcaggatcccgacacctgcagccagctctgcgtgaacctggagggtggctacaagtgccagtgtgaggaaggcttccagctggacccccacacgaaggcctgcaaggctgtgggtgagcacgggaaggcggcgggtgggggcggcctcaccccttgcaggcagcagtggtgggggagtttcatcctctgaactttgcacagactcatatcccctgaccgggaggctgtttgctcctgagggctctggcaggggagtctgccgccctgttaggacttgggcttgccagggggatgcctgcatatgtcctagtttttgggaatatccagttaacggaaccctcagccctactggtggaacaggaaccggctttcctttcagggacaacctggggagtgacttcaaggggttaaagaaaaaaaattagctgggcatggtgccacacacctgtggtcccagctactcagaaggctgaggcgggaggattgcttgagggcaggaggattggttgatcctcccacctcagcctccggagtagctgggacctcaggtgcatgccactatgcctggctaattttcttttttctttttttttttttttcgagacggagtctcgctctgttgcccaggctggagtgcagtggcaggatctcggctcactgcaagctccgcctcccgggttcacgccattctcctgcctcagcctccccagtagctgggactacaggagcccgccactgcaccaggccaatttttttgtatttttagtagagacggggtttcactgtgttagccaggatggtctcgatctcctgacttcgtgatccgcccacctcggccttccaaagtgctcggattacaggcgtgagccactgcgcccagccgctaattttcatatttttagtaaaaacagggtttcaccatgttggccaggctagtcttgaactcctgaacccaagtgatcctcctgccttggcctcccaaagtgctgggattacagacaccacacctggctattattattttttagagacagggtgctgctctatcttccagcctgtagtgcagtgcagcctccatcatagctcgctgcagccttgacctcctgggttcacgtgatcgtcccgcctaagcctctggaggagctgggagtactggcatgtgccaccatgcctggttaatttttttttttttttttttgagacagagtctcattctgtcacccaggctggagtgcggtggtgcgatcttggcttactgaaacctccacctcccaggttccagcaattctcctgcctcacccttctgagtagctgggattacaggttccggctaccaaacctggctagtttttgtatgtttagtagagacagggtttcaccatgttggtgaggctggtctcgattctcccgcctcagcctcccaaagtgctgggattacaggcttgagccaccgtgcctggcttttttttttttttttttttttgtggcaataaggtctcattgtcttgcccaggctagccttatgctcctagcctcaagtgatcctcctccctcagcctcccaaagtgctgggattacaggtgggcgccactgtgcctgttcccgttgggaggtcttttccaccctctttttctgggtgcctcctctggctcagccgcaccctgcaggatgacacaaggggatggggaggcactcttggttccatcgacgggtcccctctgaccccctgacctcgctccccggacccccaggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcaggtgagcgtcgcccctgcctgcagccttggcccgcaggtgagatgagggctcctggcgctgatgcccttctctcctcctgcctcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatgggtgcgtatccacgacgctgagggctgcagagggaatggagggagcaggaaggagcttcaggaactggttagtgggctgggcatggtggctcaaagcacctgtaatcccagcactttgggaggccaaggtgggtggatcatcaagaccagcctgaccaacatggtgaaacctcgtctctactaaaaatacaaaaattagccgggtgtggtggtgggcacctgtaatcccagctgctcgggaggctgaggcaggagaatcacttgaacctgggagatggaggttgcagtgagccaagacagccccactgcactccagcctgggtgacagagtgagactccgtctcaaaaaaaaaaaaaaaaactaaacaaaaaactggttagtggctagacaacaggatggtatcttccaagcccatggctgactcagcagctcctgggtcaagacactgtgacctgtgtcccctggcaggaagcatcgcccctgccacctgcccggtgtactctgtacctgtcaggtgacatctgctacctaagcacgtgagaggtggcatttcacagtttcagtgtggtgctgacaacccgggacgcacactgtccttgcagctacaatcaggaggtgaatgttgggtttccagcagagaacactggagaaggcacacttggtgtctggaagggaaaagcagggaagagagcatcatcagatgcctgcgggtgaaggtgggcccgctatggccagcgtccctttttatttttatttatttatttatttgagatggaatctcgctctgtcgcccagactgtagtgcagtggtgcgatcacggctcactgcaagctccgcctcacaggttcacgccattctcctgcctcagcctcccgagtagctgggactacaggcacccgccaccacgcccggttaattttttgcatttttattagagacggggtttcaccgcgttagccaggatggtctaaatctcctgaccctgtgatccacccgcctcggcctccctaagtgcttggattacaagcgtgagccaccacgcccggccccctttttattttttattttttgagacggagtctcgctctgtcgcccaggctagattgcagtggcgtgatctcggctcactgcagcctccgcctcccaggttcaagtgattctcctgcctcaacctcccaactaattaggattacaagcatgtaccaccatgcctgactaattttttgtatttttagtagagactgggtttcaccatgttggctaggctggtctcgaacccttagcctcaagtaatctgcctgcctcagcctcccaaacagcggggattacaggcatgagccactgtgcccaacccaaccctggatctcttttaaacaagacaatgctcgctgttgccacagaacaatgggtggggtacatgtggcccagtgtgtttggccacataactgccaggccagagggaaagagactctcagactgtctccactcagatacaaatgtgtgtgttgtgtgcgtgtgttctggtctcatatttgtttgttttgagacagggtgtcgctctgtcactgagtctggagtgcagtggcgcaatcagagttcactgcagcctcaaactcttgggctcagttgattctcccacttcagcctcccaagtagctggaactacaggtgaacaccactgtgcccagctaatttattttatttttagtagagatgaggtctcactatgttgcccaggctggtcttgacctcctagcctcaagcaatcctcctgccttggtctcccaaagtgctgggattacacgtgcgagccattgcgcatggcttgtgttcttgtgtttcttcctttttctttcgagatggcgtctcagtctgccacccaggctggagtgcagtggtgtgatcatagctcactgtagcctcaacttcctgggctcaagcaatcctcttgatttcagcctcccgggcctggccagcatggtgaaaccccgtctctactaaaaatacaaaaatgtagccaggcgtggtggtgggcgcctgtaatcccagctacaccagaggctgaggcaggagaatcgcttgagcctggaaggtggaggttgcagcaagccaagatcgtgccactgcactccagcctgggcaacagagacagactctgtctcaaaaaaaaaaaaaaaaaacccaaacaagccacatttggagtttggggttcccagcaggactatttcccaagcctgagcctggctgtttcttccagaattcgttgcacgcattggctgggatcctcccccgccctccagcctcacagctattctctgtcctcccaccagcttcatgtactggactgactggggaactcccgccaagatcaagaaagggggcctgaatggtgtggacatctactcgctggtgactgaaaacattcagtggcccaatggcatcaccctaggtatgttcgcaggacagccgtcccagccagggccgggcacaggctggaggacagacgggggttgccaggtggctctgggacaagcccaagctgctccctgaaggtttccctctttcttttctttgttttttctttttttgagatgaggtcttggtctgtcacccaggctggagtgcactggcgcaatcgtagctcactgcagcctccacctcccaggctcaagtgatcctcctgcctcaccctcctgagtagctgagattacagacacgtgccaccacggcagactaattttattttatttttgggaagagacaaagtcttgttatgttggcctggctggtctcaaactcagggtgcaagcgatcctcccgcctcagccttccaaactgctgggattacaggcgtgggccaccgtacccagcctccttgaagtttttctgacctgcaactcccctacctgcccattggagagggcgtcacaggggaggggttcaggctcacatgtggttggagctgcctctccaggtgcttttctgctaggtccctggcagggggtcttcctgcccggagcagcgtggccaggccctcaggaccctctgggactggcatcagcacgtgacctctccttatccacttgtgtgtctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggtgtggcttacgtacgagatgcaagcacttaggtggcggatagacacagactatagatcactcaagccaagatgaacgcagaaaactggttgtgactaggaggaggtcttagacctgagttatttctattttcttctttctttttttttttttttttgagacagagttttgctctcgtttcccaggctggagggcaatggcatgatctcggctcaccgcaacctccacctcccaggttcaagtgattctcctgtctcaggctccccagtagctgggattacaggcatgcaccaccaccatgcccggctaattttgtatttttagtagagacggagtttctccatgttggtcaggctggtctcgaactcccgacctcaggtgatctgcctgcctcggcctcccaaagtgctgggattacagacttgagccaccgcgcccagctatttctgttttctttctttcttcttcttctttttttttttctaagagacaggatctcactctgtccccaggcaggagtgcagtgctgtgatcatagctcactgcagccttaacctcctgggctcaagtgatcttcccacctcagcctcccaagtagctggaactacaggtgcacaccaccatgcccagctcatttttgtattttttttttttttgagacagtctcgttctgtcaccccggctggagtgcagtggtacaatcttggctcactgcaacctctgcctcccaggttcaagcgattctcctgcctcagcctcctgagtagttgagattacaggcatgtgtgccatcatacctggctgatttttgtatttttttttagagatggggtctcagtatgttgaccaggcttgtcttaaactcccggcctcaagtgatcctcccacttcagtctcccaaagtgctgggattacaggcatgagccactgcggccggtttgttttcttttttttttcgttttttggagacggaatttcacctttgttgcccaggatggagtgcaatggcacgatatcgcctcaccacaacctctgcctcctgggttcaaaccattttcctgcctcagccttcttagtagctgggattacaagcatgtgccaccacgcccggctgattttgtatttttagtagagatggggtttctccatgttggccaggctggtctcgaactcctgacctcaggtcattcgcccacctctgcctcccaaagtgctgggattacaggcgtgagccaccgtgcccggtggtttgtattctttttactgagagtcgtgaaaggcagtgatcctctgtcacatgtgatcttggctctcaggggacatttggcaatttctagagattttttggttgtcacaagtcaatggggaagactgttggcatttagtgggtagaggctggtgacgctgctgaacacccagaacagggaagtagcaggccctagatagagccatcgtggggaaaccctgctctaaggaaatggcgctattttataaccccacgttcctggcatgattaccaacagccaaaagtggagtccccccaagtgtgttcgtccatttgcattgcagtaaaggaatagctgaggccgggtaatttataaagaaaagagatttaaactgggtatggcagtttatgcctataatcccagaactttgggaggctgaggcaggaggatcgcttgagtccaggagtgtgagaccgagaccagcctggccaacatgacgaaactctgtctctacaaaaaatacaaaaagtaggccaggcacggtggttcacgcctgtaatcccagcactttgggaggccgaggcgggcggatcacgaggtcaggagatcgagaccatcctggctaacacggtgaaaccccgtctctactaaaaatacaaaaacaaaattagccgggtgtggtggcaggcgcctgtagtcccagctactcgggaggctgaggcgggagaatggcgtgaacccgggaggcggagcttgcagtgagccaagatcgcgccactgcactccagcctgggtgaccgagttgagactccgtctcaaaaaaaaaaaaaaaaaaaaaaatacaaaaagtagccaggtgtggtggcaggcacctgtaatcctgggttctcgagaccgaggcatgagaattgcctgaccccaggaggtggaggctgcagtgagccaagatcatgccactgcactccagcctgggcgacagagtgggactctgtctcaaaaaacaacaaaaaaaaagttctggaaatggatggtggtgatggtgatacttccacaacagcgtgaatctgcttaaggccaccgaactgtgcactcacaaatagtcgagatggtacattttatgttatgtgtatttcaccacaattaaaaactagttgtgggccaggtgtggtggttcatgcctgtaatcccagcactttgggaggtcagagggaggtggatcatgaggtcagcagttcgagaccagccaggccaacatggtgaaaccccatctctactaaaaatacaaaaattagccaggcgtggtggcacatgcctgtagtcccagctacttgagaggctgaagcaggagaatcgcttgaacctgggaggctaagattgcagtgagccgagatcgtgccactgcactccagcctggacgacagagtgagacttcgtctcaaaaaaaaaaccaaaaaaaaaattagctgtgggtcaggcactgtggctcacgcctgtaatcccagcactttgggagaccgaggtaggtggatggcctgaggtcaggagttcgaatccagcctggccaacatggtgaaagcccgtctctactaaaaatacaaaaaattagtcaggtatgttggcacacctgtaatcccagctactcgggaggctgaagcaagagaatcgtttgaacccaggaggtggacgttgcagtgagccgagattgggccactgtactccagcctgggcaacaaaagtgaaactctgtctgaaacaaacaaacaaacaaacaaacagacaaacaaaaaaactagttgtggagagagggtggcctgtgtctcatcccagtgtttaacgggatttgtcatcttccttgctgcctgtttaggacaaagtattttggacagatatcatcaacgaagccattttcagtgccaaccgcctcacaggttccgatgtcaacttgttggctgaaaacctactgtccccagaggatatggttctcttccacaacctcacccagccaagaggtaagggtgggtcagccccacccccccaaccttgaaacctccttgtggaaactctggaatgttctggaaatttctggaatcttctggtatagctgatgatctcgttcctgccctgactccgcttcttctgccccaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacaggtgtggcacacgccttgtttctgcgtcctgtgtcctccaactgccccctcctgagcctctctctgctcatctgtcaaatgggtacctcaaggtcgttgtaaggactcatgagtcgggataaccatacttttcttggatggacacatcagcaccgggcttgacatttacccagttcccctttgatgcctggtttcctctttcccggccccctgaagaggtgatctgatttctgacaggagccctgagggaggaaatggtcccctttgttgacttttctttttctttatttttttcttttgagatttgctgtcacccagcctggaatgcagtggtgccatcttggctcactgctacctctcccactgggttcaagcaattctcctgcctcagcctcccaagtagctgggattacaagcatgcgccaccatgcctggctaagttttgtatttttagtacagacagggtttctccatggtggccaggctggtcttgaactcctgacctcaggtgatcctcccacctctgcctcccgaagtgctacgattacaggcatgagccaccgcgcccatccccctttgttgacttttctcatcctctgagaaagtctcagttgaggccagcacctccctcaagtgaattgaatctcccttttgaacaacaacaaataacaatatgacccagacgtggtggctcacacctgtggtcccagctactcgggaggctgaggtgtgaggattgcttgagcccaggaggtcaaggctacagagagctataatcacaccacttcactccagcctgggggacaaagtgaaaccctgtctgaaaaaaacaaaaaaagaaaaaggaaaaagaaacaatacgatcacaaagtagatattcatagtgtttattttcagtactcttttttttttttttttttttttttgagacggagtcttgctctgttgcccaggctggagtgcagtggcacgatcttggctcactgcagcctctgcctcccaggttcaagcgcttggctcactgcaacctccgcctcctgggttcaagcgcttcttctgcctcagcctccccagtagctgggactataggcacgtcccactacgcccagctaattttttgtattttttagtagagatggggtttcactatgttagccaggatggtctcgatctcctgacctcgtgatctgcctgccttgggctcccaaagtgttgggattatgggcatgagccactgcacctggccttttttttttttttttttgagatggagtttcgctcttgttgcccaggctggagtgcaatggtgtgatctcggctcactgcaacctctgcctcctgggttcaagcaattctcctgcctcagcctcccgagtagctgggattacaggcacctgccaccacgcctggctaatttttgtacttttagtagagacggggtttctccatgttggtcaggctggtctcaaactcctgacctcaggtgatccacccacctcggcctcccaaagttctgggattacagacatgagccaccgcgcctggccgtgtctggccttttttagttatttcttttttttttttttttttttttgagacagagtcttactccgtcgcccaggctggagtgcagcggtgcgatgtctgcgcactgcaagctccgccccctgggttcatgccattctcctgcctcagccttctgagtagctgggactgcaggcgcctgccactacgcccggctacttttttgtatatttagtagagatggagtttcactgtgttagccaggatggtctcgatctcctgactttgtgatccgcccgcctcggcctcccaaagtgctgggattacaggcgtgagccaccatgccaggcttttttttttttttttttttttgagacggagtcttgctctgtcgcccaggctggagtgcagtgccatgatctcagctcactgcaagctccacttcccaggctcacgccattctccagcctcagcctcccaagtagctgagactacaggggcccgccaccacactcggctaatttttttgtatttttagtagagacggggtttcaccatgttagccaggctggtcttgaactcctaacctcaggcgattcacctgcctcggcctcccaaagtgctgggattaaaggtatgagccacctcgcctggtgtgagccacctcgcccagcctgagccacctcacccagcctaagccactgtgcctggcctgattttggactttttaaaaattttattaataattatttttgggtttcttttttttgagacagggtcttactctgtcatccaggccatcctgtctgtctgtcatcccagtgatgggatcataccttgctgcagcctctacctcctgggctcaagcgatcctcccccctcagcctcctgagtagctgggagtacaggtgtgcaccaccacacctggctaatttttttttttttttttgtatatagagatggtattttgccatgttgaccaggctagtcttaaactcctggactcactcaagagatcctcctgccttggcctcccaaggtcatttgagactttcgtcattaggcgcacacctatgagaagggcctgcaggcacgtggcactcagaagacgtttatttattctttcagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaaggtaaagactgggccctccctaggcccctcttcacccagagacgggtcccttcagtggccacgaacattttggtcacgagatggagtccaggtgtcgtcctcactcccttgctgaccttctctcacttgggccgtgtgtctctgggccctcagtttccctatctgtaaagtgggtctaataacagttcttgccctctttgcaaggattaaatgggccaaatcatatgaggggccaggtccttcaggctcctggttcccaaagtcagccacgcaccgtgtgggtcccaaaattttatcaaggcacattcgttgcctcagcttcaggcatctgcccaaaaaggccaggactaaggcaaggagagggagggattcctcagtactcagcttttcacagaggctccaaaaggctaaggaatccagtaacgttttaacacaattttacaatttttttttttgagacggagttttgctcttgttgcccaggctggagtgcagtggcacgatctcggctcactgcaacctctggctcccgggttcaagcgattctcctgcctcagtctcccgagtagctgggattacaggcatgcgccaccacgctcggctaattttgtatttttagtacagaaggggcttctctgttggtcaggctggtcgtgaactctcaacctcaggtgagccacccgcctgagcctcccaaagtgctgggattacaggtgtgagccaccacgcctggccttttttttgagacagagtctcgctctcgcccatgctgtactgcagtgacgcagtctgggctcactgtaacctccgcttcccaggttcaagtgattcttctgccgcagcctcccatgtagagtagctgggattacaggcacccgccaccatgcctggctaattcttgcatttttagtagagatggggtttcacagtgttggccaggctggtctcaaacttctgacctcaagtcatctgcctgccttggccctgccaaagtgctgggattatagatgtgagccaccgcgcctggcctacagtttattctttggtggctcacacctgtaatctcagcactttgggaggccaaggtgggagaatggcttgagcccaggagttcaagtccagcctgggcaacatagcaagaccctatctctactacaaaataaataataaataaactaattttttttcttttaaaacccaactattcaacatggcaatgcaatatattaaaaaaattttttttttctttgaaacggagtctctcactgtcacccgggctggagtgcagtgtcgccatcttggctcactgcaacctccgcctcccaggtccaagtgattctcctgcttcagcctcccgagtagctgggattacaggcacccaccaccatacccagctaatatttttgtatttttagtagagatggggtttcactatgttgggcaggctggtctggaactcctgacctcgtgatctgcccgaggatcggcggcctcccaaagtgctggggattgcaggcatgagccaccgtgcccagccaaaacttttttatttttatttttttgggacacggtctcactgtgtaccccagactggagtgatagagtgctgtcatggctcactgcagcctcaacctccctgggctcaggtgatcttcctgcttcagtctcccaggtagctgggactacaggcatgagccaccacacccagctaatttttgaatttttttgtagagacagggtttcaccttgtggcccagacttgtctctaactccagggctcaagcgatctgcccaccttggcctcccaaagtgctgagattaatgcaatttaaaaaattttttggccaggcctggtggctcatgcctgtattcacaacaccttgggaggcaaaggtgggcagatcacttgaggtcaggagttcgagactagcctggccaacatggtgaaaccccctgtctactaaaaaaatacaaaaattacctgggcacagtggtgggtgcctgtaatcccagctacttgggatgctgagggtggagaattgcttgaacctgggaggcagaagttgcagtaagccaagatcatgccactggactccagcctcagtgacagagcaaaactctgtctccaaaaaaattgttttttttttttttttttcaaatcatcacactacagccaaggcctggccacttacttttgtaaataaagttttattggagccagtggaccagtgaggccgaatcttgcaggtgtaagatcacagtctatccttgaaaattttgatattttgttcattgggtggtttttcattaatttaaattttaaaaaataacatattaaaggctggtgtggaggtgcacgcctgcagtcctagctactcccagaggctgaggcgggagacttgcttgagcccaagagttgaagtccagcctgggcaacatagcgagacccccatctctaaaaataaaaataatgcattagaatattattggattcctgggcagggcacagtggctcacacctgtaatcccagcactttgggaggctgaggtgggtggatcacctgaggtcaggagtttgagaccagcctggccaacatggtgaaaccccgtctctactaaaaatacaaaaattagccaggcgtggtggcaggtgcctgtaatcccagctactcgggaggctgaagcacgagaatcgcttgaatccaggaggcggaggttgcagtgagctgagattgcgccattgcactccagcctggaggacaagagtgaaactccattcccctctgcaaagaaaaggaatattatcagattcctaagctttttggctccccctttagtttgggggctggggtggtgagtgtctgacctggcctcactgtcctccctggatgtgatgagacccaggtgtgggtcaggatgtcattcgtttgtccaccagagggcgcccaaactgctttgagctgctgggaaatggtgctcctagacttttagcaaacaaacaaaaaaaaatggcacatcggcaaatttcagaccattctttttttttttttttttggttccagagtagctgaaatctttgttcagttacaagcaggataaaatggaaactgcctgggagaggctgagaaaccttcttgcttgggggaggtggggcactgctagaattaatcgcttcacagaccagcccatccaggactcctcaaatttggcaaaaaagccattcattcattcattcatttatgtagagacgagggggatctggctatattgcctagattggtctcaaattcctggcctcaagtgatcctcctgccttggtctactaatgtgctgcgattacaggcatgagccaccgtgcctagctctagtggacttgaaatgttgccttgcccagggcccttatgttgaatggcccaggtccacttgtatggttctgtaccaaggttaaccccatcccataatgcctgggacagttgatgcaggacaatcagcttctgtgccattcaacctcaggactgagcatgctgggcattgtggggtccgaaggtggctcccctgtccccttcaaaataccctctttttcttttcttcttttttttttttttttttttttgagacgaagtcttgctctgttgccccagctagagtgcagtggtgcgatctcagctccccgcaacctctgcttcccgggttcaggcgattctcctgcctcagcctcctgagtagctgggattacaggtgcccaccgccacagctggctaatttttgtatttttagtagagacagggtttcaccgtgttggccaggctggtcttgaactcctgacctcaggcaacctgcccacctcagcctcccaaagtgctgggattacaggtttgagccactgggcctggccttttttttttttttttgagagggagtctcactctgttgcccaggctggagtgcaatggcgcgatcttgactcactgcaactccatttcccgggttcaagtgattctcctccctcagcctcccaagtagctgggattacaggtgcatgccaccacggccagctaattttgtatttttagtagagacagggtttcactatgttgatcatgctggtctcaaactcctgaccttaggtgatctgcccgccttagcctcccaaagtgttgggattacaggtgtgagccaccgcgcccagaccaaaatatgctcattttaataaaatgcacaagtaggttgacaagaatttcacctgcaaccttgtcaaccacctagaataaaagcctctgcagccctcccctaaagactcatcaatgtgaggctcaagaaccttcttaggctgggctcggtggctcatttctgtaatccctgcactttggaaggctgaggcaggaggatctcttgaggccaggagttcaagacaagcctgggcaacatagccagacctctgtttctatcccccacaaaaagaaccttcttaaaccggaattgagtcctacaacctcgataactcacaaataagcccgtgtggcctctcacagacttgggaagttctccaagtgtccagggagatgtgccaggcgctttcctgccgtgaccaccgtcctctgcctgctccatttcttggtggccttcctttagacctgggcctcactcttgcttctctcctgcagctctgggcgacgttgctggcagaggaaatgagaagaagcccagtagcgtgagggctctgtccattgtcctccccatcggtaagcgcgggccggtcccccagcgtcccccaggtcacagcctcccgctatgtgacctcgtgcctggctggttgggcctgttcactttttctcctggacagggaacagccccactggtgtcctttatcacccccacggcctctcctggcttggggctgacagtgacaagatcagacagctaaggggtcagatggaggatgtggagctgggtcccgtgctgtggaatagcctcaccgagatttgagtgccttctggggaactggttcccttgcagggggctgtgtggagaggcgcgctctccctgcctcacccatgctcatcctaactcggttaccatcacatctcttttttctttttttcttaaattttaagaaaaaagaaatttaatttttttgagagacagagtcttgctctgtcacccaggctggagtgcagtggcaccatcatgcctcgctgcagcctcaatgtctgggctcaagcgatcctcccacctcagcctcctgagtagctggtgcaagccactataccccacttcctatttcttaaaaagtcacagccctgtgtgtggctaatcctggacagaaatctagaagaagtcagctacttctggggcgtggctcacccagtgggcttcaggttagatatttcttatacttatgaggctgggtgtggtggcttatgcctgtaatcccagcactttgggaggctgaagtgggtggattgcttgggctcaggagttcgagaccaacctgggcaacatggcgaaaccctgtttctagaaaaggtacaaaaattagctgggcaggtggcacgtgcctgtggtaccagctacttgagggcctgaggcaggaggatcgcttgaacctgggaggtcgaggttgcagtgaactgagatcatgtcactgcactccagcctggtgacagagcaagaccccgtctcaaaaaaaaaaaaagaaagaaaaaaattcttatgcatagatttgcctcttttctgtttgtttgttttgagatggagtctcgctctgtcgcccaggctggagtacagtggctcaacctcggctcactgcaacctctgcctcccgggttcaagcaattctcctgcctcagcctcctgagtagctgggactacaggcgcccgccaccatgcccagctaatttttgtatttttagtagagactgactgggtttcatcatgttggccaggctggtctcgaactcttgacctcatgatccgcccgcctcagcctcccaaaatgctgggattacaggcgtgagccaccaggcccaggccgcaaggcgatctctaaacaaacataaaagaccaggagtcaaggttatggtacgatgcccgtgttttcactccagccacggagctgggtctctggtctcgggggcagctgtgtgacagagcgtgcctctccctacagtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcggtgagtgaccctctctagaaagccagagcccatggcggccccctcccagctggaggcatatgatcctcaagggaccaggccgaggcttccccagccctccagatcgaggacagcattaggtgaatgcttctgtgcgctcattcagaatgtcagcggacaatggccttggtggtgtagaggaatgttggataagcaaatagagagctccatcagatggtgacagggcaaagaaagtcaaaaggagttcagaggccgggcgcggtggctcatgcctgtaatcccaggactttgggaggccgaggctggcggatcacctgaagtcaggagtttgagaccagcttggccatcatgacaaaaccccgtctctattaaaaatacaaaaaattagccaggcgtgggagtgggcgcctgtaatcccagctactcgggaggccgaggtagaaaaatcgcttgaacctaggaggcagaggttgcagtgagccgagatcgcgccactgcattccagcccgggaggcaagagcaaaactccatctcaaaaaaaaaaaaaaaaggagttcagaggcccggcatggtggttcacacatgtgatcccagaacttggggaggttgaggcaggagaatcacctgagctcagagttcaagaccagcctgggcagcacagcaagaccccatctctgcaaaaaataaaaatttagcccagtgtggtgatgagcgcctagttccagctactagggaggctaaggcaggaggattgcttgaggctaaggtaggagattgagactgcagtgacttgtgattgcgtcactgcgctccagcctgggtgacagagcaagcccttgtctcttaaaaaaaaaaaaaaattcaaagaagggtttccagagggccaggagggaggaagggagaggaggtgttttatttttttgcttttattttttattttgagacagagtctctctctgtcacccaggttggagtgcagtgctgtgatcttggctcactgcaacttctgcctcctgggttcaagcaattcttatgcctcagcctcagcctcctgagtagctgggattacaacactatgcccgggtaatttttgtatttttagtagagacgaggtttcgccatgttgcccagactggtctcgaactcctgacctcaagtgatccacccgccttggcctccccacgtgctgggattgcaggcgtgagccactgcgcccgccttgatctttacacaaggggtttagggtaggtagccttctctgaaccaggagaacagcctgtgcgaaggccctgaggctggaccgtgcctgttgggtttgaggccgttgtagctggagcaaacagagagaggggtaaaaaggcaggaggctaccaggcaggttgtgcagagccttgtgggccactggggaggactttggcttttgccctgagagcggtgggaagtgactgaatccggtactcaccgtctccctctggcggctcctgggggaacatgcttggggatcaggctgggggaggctgccaggcccaggaggtgagaagtaggtggcctccagccgtgtttcctgaatgctggactgatagtttccgctgtttaccatttgttggcagagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgta

SEQ ID NO: 4 - DNA, 42802 bp, Homo sapiens
  source 1..42802: mol_type = genomic DNA
ctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcaggtaaggcttgctccaggcgccagaataggttgagagggagcccccggggggcccttgggaatttatttttttgggtacaaataatcactccatccctgggagacttgtggggtaatggcacggggtccttcccaaacggctggagggggcgctggaggggggcgctgaggggagcgcgagggtcgggaggagtctgagggatttaagggaaacggggcaccgctgtcccccaagtctccacagggtgagggaccgcatcttctttgagacggagtctagctctgtcgcccaggatggagtgcagtggcacgatctcagctcactgcaacctccgcctcccgggtttaagcgagtctcctctctcagcctcccgaatagctgggattacaggcgcccaaccaccacgcccgcctaatttttgtatttttagtagagacgggttttcaccattttggccaggctggtctcgaaccccgacctcaggtgatctgcccaaaagtgctgggattacaggcgtcagccaccgcgcccggccgggaccctctcttctaactcggagctgggtgtggggacctccagtcctaaaacaagggatcactcccacccccgccttaagtccttctgggggcgagggcgactggagacccggatgtccagcctggaggtcaccgcgggctcaggggtcccgatccgctttgcgcgaccccagggcgccactgccatcctgagttgggtgcagtcccgggattccgccgcgtgctccgggacgggggccaccccctcccgcccctgcccccgcccctttggcccgccccccgaattccattgggtgtagtccaacaggccaccctcgagccactccccttgtccaatgtgaggcggtggaggcggaggcgggcgtcgggaggacggggcttgtgtacgagcggggcggggctggcgcggaagtctgagcctcaccttgtccggggcgaggcggatgcaggggaggcctggcgttcctccgcggttcctgtcacaaaggcgacgacaagtcccgggtccccggagccgcctccgcgacatacacgagtcgccctccgttatcctgggccctcctggcgaagtccccggtttccgctgtgctctgtggcgacacctccgtccccaccttgtcctggggggcgccctcgccccaccagccccgatcaagttcacagaggggcccccggccaccctcaaggcctcggttccttacgaggttgaaacgttgcctcagaatctccccgcccctccttggtctgcagccgagatcttcagccacggtggggcagctatcccccgggaccgaccccctggggtggcctcgcttcttcagaggctgtgaatggcttcggttcagctgtccaagcggcgatttttcctctgggtgaaatggattagattttagatttccacaagaggctggttagtgcatgatcctgagttagagctttttaggtggctttaaattagttgcagagagacagcctcgccctagacaacagctacatggccctttccctcctgagaaccagcctagcctagaaaaggattgggattgcctgatgaacacaaggattgcaggaaactttttttttaattggcaagggggttggctttgactggatggagagctttgaactgccttgaaattcacgctgtaactaacacaccagtttcctctgggaggccagagagggagggagggtgtaatgaaatacggatgattgttcttttatttttatttacttatttattttttaactttttgtagagatgaggtctcgcttggttgctcaggctggtcttgaactcctggcctcaagcgatcctcctacctcagcctcccaaagtgttgggattacaggagtgagccaccgcgccccaccggggatgatgatgattgcaaacattctgccactcagttttacaaaagaaagagaggcactggattaatgtgtatctcactcaccaatcaacctcttccttaagataaaatgttaaggaagtcttaggcaaggccttgtttgttcatcactttagtttctctctcccgggatggctgagaatgtgatgtttcctctgttgtcaaggagactacacccctgatgttttcctccagacttctgagagctggtgtgtgtttctagcactttctagctgcaccacctcacgctgtagctggcttcaaggcatatccaggggggagtttcttgtccatttcctttacaaagggaagttgttggaatctgaaccgcaagccttcacttagaccaaaatcaggcaacagcggtgagcgcagctccaaacgtgtcaatgactcacccaaatttgagtaagggagttggctgctttaacgagccgcagggtgattcccttgtcatttccggaaatacctatcttccagggaacactgggaaaaaacagggagacctttgttgagacagaaaacctgtaggggaattctgttcctcattcctgctcttatctgtagacttcctccctgataagatccaattctagatgggtcggttgctccttgctttgatgggtgctttgatgggctttattattattattattattattattattattttgatgggctttttgatgtcccttttccttccacactctgtcccaactgtcaagcaaatagccttttgttgctaagagactgcagatgtaaccgaccagcagcaaacagtgagtcaggctctctcttccggaagcaaaatcaattgctgagatcactctggggaaaatacccaccttatttggaaagaagcactgatcaattgatgtctattttttttttttttgagttggagtctcgccctgtcacccaggctggagtgcaatggcataatctcgcctcactgcaatccccgcctcccgggttccagcaattctcctgcctcagcctcctgagtagctggaattataggcgcctgccacaacacccggctaatttttgtatttgtagtagagatggggtttcaccacgttggccaggctggtctcgaactcctgacctcgtgatccacccgcctcagcctcccaaagtccaaggattgcaggcgtgacccactgtgccagccaatcaattgatttctcattcattttcagctggctctgttcccttaagccaggggattttcgtttgtttgtttccccttcaaggaaatgattctagctacagttttgatttccttgtacaactgttttcagtagcacagggaaagaaaacatcgaaagcattcaccacctcatttgtgtgctgggggaaaaagcagaaatgtgtattctctttttttgtttcgatgaccttgttcctgacttgttactcgtgacttgagagatcagagggctagaggactagaatttatagaggtgttttttttgtttgtttatttttgttcgagttgcccaggctggagtgcagtggcgcaatctcggctcactgcaacctctgcctcccaggttcaagcgattcttcggcctcagcctcctgagtagctggaactacaggcgcccgccaccacacccagctaatttttgtatttttcagtagagatgggatttcaccatattggtcaagctggcctcgaactcctgacctcgtgatccacccgcctcagtttcccaaagtgctgggagtacaggcgtgagccgccgtgcccggcctttttgtgtttttgtgtttttgagaggagctcattgctttttaggcttccctagcgtgagaaaatctggggatccatgctctagtttacttcctttttttttttttttttgagatggagtctcgcttagattgcctaatctcagctcattgcaacttctgcctccggggttcaagggattctcgtgtctcagcctcctgggtagctaggatacgggcacccgctaccatgcctggctaattttgtacttttagtagagacagggtttcgccacgttggccaggctggtctcgaactcctgacctcaggtgagccgcctgccttggcctcccaaagtgctgagattacaggcgtgagccaccgcgcttggcctaatttgcttttcctgaaattcaaatggtctaatatgaaaaacgccaaccttgcttgaaagaataagaaagaggtgcggtttcgttgggccgttgatgtttggaacaggactggttttgtccccttgctcggaaagggcagcaactgtgaggacagctccctgacgtgctctcactcagcactgttccgttcctgagcactgtccccactagctaggccaagggagctcatttggcaggcaactgctgtctggctgcgcctgtggcagtaaaatctgcctttattttttggaggcagggtcttgccctgtcgctcaggctgaagtgtgcagttatagctcactgcagcctccagcttctgtactcaactgatcctcctctctcagcctcctgagtagctgggactatacgcacgtgttaccactcccacctcagtttgtttgtttatttatttatttatttatttattgagatggagttttgctcttgctgcccaggctggagtgcaatggcgcgatctcggctcaccgcaacctccacctcctggttcaagcgattctcctgcctcagcctcctgagtagctgggattacaggcatgcaccaccacgcccggctaattttgtatttttcgtagagatggggtttctccacattggttcaggctgttctcgaactcccaacctcaggtgatccacccgcctcagcctcccaaagtgctgggattataggcgtgagcccccgaacccggccactcccagctaagtttaaattttttgtttgtttgttcgtttgtttttattttttgagacagagtctcccgcccaggctggagcgcagatcactgcatccttgacctcccaggcttaagccatcctccccactcagcctcccaagtagctgggattacaggtgtgtgccactatgcttggctaagttgtgtattttttgtagagatggggttcaagggattctcgctttgttgcctcggttggtctcaaactcctgggctcaagcagtcctccctcctcagcctcccaaggtgctggggaaatccacttttgaaacattgtctggagagttgcccaggtggtagatcacagaaataggtcatcgtggggtccttcccatgggtgcagtcttgagccacctgtggccagcaaatatttggagaataatagtcaggggagagcttgaggtccagggaaaggttttgtttttcttcagggaaaggtttttattgttctttatccctccttaaaggaccttcaggtgttactgacattcccggtctacccagtggcacatttagtttgtaagctgggccctcgtacagaggtagggaggtgagagcattggattagtggtcaccaaagctgcggtcacctagtggggtgatcagaggctcctcccttaagatcttgattgccaacgcctctggcccaactttcctttttatttatcgcaagcctcctggaatctcaattgctttttgcccacccggtgtgtcagcacaagaaatgagtcatttcctcctttaagcacagttgaaattgagctgtgagtcagtgaggtgtgtacgatattgtcaaagcggggtgtgtacagtattgacagatctgtagttgggcaagagaattatcagagtttgtgaccacagcagattccaaagctcgactcattttcttctctcttccttcccttttttcttttctttttttttttttttttgacagagtctcgctctgttgcccaggctggagtgcagtggcacaatctgggctcactgcagcccctgcctcctgggttcaaatgattctcatgtttcagcctcccgagtagctgcaattacaggcattcgggttcaagtgattctcctgcctcagccacctgagcagctgggattacaggcgcccgccaccacgcccggctaatttttgtatttttagtagagacggggtttcaccatgttggccaggctggtctcgaactcctgaactcaggtgatccgcccacttcggcctcccaaagtgctgagattacagacgtgagtcaccgcgcccagcctgttctgttctttaattctcaaaacaccctctaggaagtagagactgccattctcccccattttacagatcaggaaactgagtcccagaaggatttagtcagttacccaagttgttctagttaaatggcctggaaagccagtgaagcccaggattgtctatctaacccccttactactctaactttcagggaatccacatgaatgtgctgggtcaaccatcaaagttgaaatggataaagggggctggatgcggtggctgatgcctgtaatcctagcactttgggaggccgagatgggtgggtggattgcttgagcccaagagtttgagaccagcctgggcaacatagtgagacacctgtctctgcaaaaaataaataaaaagttagctgagtgtgatggtgcacccctctagtcacagctgttgagttaggcttaggcaggaggatcgcatgaacctgggaggtggaggcggccgtgagcctcagtcatgccactgcactccaacctgggcaacagagtgaaagccggtgtccgaaagagaaagaaaaaaagacatagatacatcttttaaagttaggttgtatgttaattacctacaactcagtttcaactgtgcttaaaggaggaaatgactcatttcttgctacatatcaaattagcccaaaatgtagtggcttaaaacaacacatttatgatttctcagtttttgcgtgtcaggaatttggaagcagcacagctagacggttccagctcagggtctctcatgaagttgcaatcaaaatattggcaggagagaaaaacatattttcagaagctgcaggcataggaagacttggctggggttgaaggatccacttccaagatggcgcactcagtggctcttggctggaggcctcagttccctgctgcgtggagctctccctccagctgcttgagtggactcatgacatgcagctggcctcccctggagcagtcgatccaacaatgagcatggccatgaactaggctcagaagccactccctgtcgtctctacattttcctatcagaagcaagtcattaaaagtccagtgccactccaggggagacgaattaggctctgccttctgaaaggattatcacagaagatgcggtcctatattctttttttaaaattattcttttttttattttgtagagatggggtcttggtatgttgcctaggccagtctggaattcctgggctcaaacaatcctgtctctgcctcccaaagtgttgggattacaggcatgagccactgcacctggtcatgtggtcatattttctttttcttttttttttttttttgagacagagtctctgtcgcccaggctggagtatggtggcgtgatctcagttcactgcagcctccgcctcccgggttcaagcgattctcctgcctcagcctcctgagtagctgggattacaggcgcccgccaacatgcccagctaatttttttagtagagatggggtttcaccatgttagccaggatggtctcgatctcctgatttggtgatccgcccaccttggcctcccaaagtttcaaccatcgatcagaacttattgatgtacttatgtagctaggcacggtggcgcgtgcctgtaatcccagctacttggaagggttaaggcaggagaatcgcttgaacctgggaggcagaggttacagtgagtcaagatcataccattgcactccagtctgggcaacagaatgagactctgtctcaaaaacaaaaaacaaacccttgtatgtgattttcctggatagcatctgttacatcttcacaaagataaaaagtcagacttggctgggcatggtggctcacacctgtaatcccagcactgagaggctgaggcaggcagatcacttgaggtcaggaatttgagaccaggctgggcagcatggtgaaaccccgtctctacaaaaaatacaaaaattagccgggtgtggtgtcacgcacctgtattcccaagctactcaggaagctaaggcaggagaatcacttgaacccagaggtggaggtttgcagtgagttgagattgtgccattgcactccagcctgggcgacagagtgagactctgtgtcaaaaataaaataaaataaaattttaaaaaaggcagatttttttttcttcttggtattgttaccttattatagtaataataagtgcatagtgcatgctgagataagcaatcataatttgttattgcggccgggcatggtggctccagcctataatcccagcactttggtcaggagttcaaggccagcctggccaatatagtgaaactccatctctactaaaatacaagaaattacctgggcatggtggcagttgctggtgatccccagctacttgggaggctgaggcaggagaatcgcttgaacctgggaagcagaggttgcagtgagccaagattgcaccactgcactccagcctgggtgacagagtgagactctgtctgaaaataataataataataatttgttattgcttttattgccttagtttacatagggaatcaaagtttatactttgatttataaaagttgctttgattctagttcacagaaccagaatctttcatataaaggtattagagggcccagtgtggtggctcatgcctgtaatcccagcatattgggaggctgaggagggaggatcactttaggagtttgaggccagcctaggcaacatagtgagaccttgtctctacaaaaaattccaacattagctgggcatggtggcatgtgcctgtagtcccatttatttggggggctgaggcaggaggatcacttgagcccacgaggttcaatccaggttgcagtaagccatgatcctgccactgcactccagtttgggtaacagagcgaagctatgtctcaaaaaaagaaaaaaaaagtattctaaatccaaatttaatatataaaactaaatgcaggccaagtgtggtggcatatacctataatcacaacactttgggaggctgaggtgggaggattgcttgagcccaagagttcaagaccagcctaggtaacacagtaagaccccatctctacaaaaagtagaaaaattagcctggcatggtggtgagtgcttttaatcccaactacttagggggctgagatgggaagattgcttgagcctcagagtttgaggctgcagtgggccgtgatcgctccactgatcgctctaaagtgagaccctgtctcaaaaaaaaagaaaatagaagaaaactaaatacattcaataagactttgatctcttttccaaggtgtaaatatattttgggaaattttccagttactttgttctcattttaatgtaataatctaagtcttggttttctaaggaaaagttttctcttattatatcttttgttaatgtttctctcccatttcttttgatctgatcttcagatacatgattatcttcactgctaaatttgtgttctctggcctctacatttataatttctcataattctttatctaagtatttcttccctacctactgaagaaaactcaagttttcttccaccttaatgattatgctgtgtctgtgagttttcttcatgactctttacagtacaagttttttgtttttgtttttttaatggtcagatggatagaacaacacaggttttgtttgttttgttttaacttttaaaaaaattataatagataaagggtctcactacgttgtccaggctgatctcatactcctgggctcaagcaatccacccacctctgcctcccaaagtgctgggattacagtcatgagccaacatgcctgggcagtacaggttttttttgagacggagttttgttcttgttgccgaggctggagtgcaatggcacaatcttggctcaccacaaagtctgcctcccaggttcaagtgattctcctgcctcagcctcctgagtagctgggattacaggcatgtgccaccacgcccagctaattttgtatttttagtagagacggggtttcaccatgttggccaggctggtttcgaactgctgacctcaggtgatctgcccacctcggcctcccaaagtgctgggattacaggcatgagccaccatgcccagctgtagtacaggttttaatatgctaaatactcttcctttctttattaatgtgcatggaagttctaatatttttttcccataccccagagagtccatattttggaatcaacaacactagcctttgttgacaagtgtctctcttgggttccttctttgtgtcctccactgaattttggggttcataaaatttcatttgttgtgcttgcttaattccctgggaatcagactgttcctgatcggatgacatttctggttaattctttagttggcaggaaatagacacaggaaacgtggtcagtttctgattctggcgttgagagaccctttctccttttcctctctctcagtgggcgacagatgcgaaagaaacgagttccagtgccaagacgggaaatgcatctcctacaagtgggtctgcgatggcagcgctgagtgccaggatggctctgatgagtcccaggagacgtgctgtgagtcccctttgggcatgatatgcatttatttttgtaatagagacagggtctcgccatgttggccaggctggtcttgaatttctggtctcaagtgatccgctggcctcggcctcccaaagtgctgggattacaggcaccacgcctggcctgtgacacgattcttaacccctttttgatgatggcggctggaaaagtggccagtggattttgatgtattcaatcatgaattaggaggtggggagagaatgaattattggagctttccttaaagccattaaatggctctattgttttttcaattgatgtgaatttcacataacatgaaattaaccagctcagtggcattaatacatctgcaatgctgtgtggccaccacctctatcttgttccaaaactttgcataacctaatgtctttttttttttttttttttgagacggagtctcgttccatcacccaggctggagtgcagtggtgtgatctcagctcactgcaacctccgcctcccaggttcacgccatcctcctgcctcagcctcccgagtagctgggactacaggcaccctccaccacatccggctaattttttgtatctttagtagagatggggtttcaccatgttagccgggatggtctcgatctcctgacctcgtgatccacctgcctccgcctcccaaagtgctggcattacaggcgtgagccaccatgcccggcctatttttttttttaagagatggagtctaattctgttgcccaggctggagtccagtggtaccatcatacttcactgcagccttgacctcttgggctcaagtgattctcttgcctcgaactcccaaagtattgggattacaggtgtgagccaccgcactcagcctaatgtccagtttttaacaagctccatttaaatgccctccgttttgacccataaaggggtaggcttggccgggcacaatggcttgtgtctgtagtcccagctacttgggaggctgaggcagaaaggcagaaagattgctttataaagcccaggagtttgagggccacctgggtggcatagctagacctcatctctaaaaaataagtaataaataaatatttgtttttgtttttttctttttcttttcttttttttttttttttgagacggagtcttgctctgttgcccaggctggagtgcagtggcgcgatctcagctcactgcaagctgtgcctcctgggttcatgccattctcctgcctcagcctcccgagtagctgggactacaggcgcccactaccacgcccagctaattttttgtatttttagtagagatggggtttcaccacgttagccaggatggtctcaatctcctgacctcgtgatccgccagctttggcctcccaaagtgttgggattacaggcgtgagccactgagcccgccccatatgtatgtatatatatatttttttaaaatgggagaccaggcatggtggctcatgcctagaatcccagcactttgggaagctgaggtaggcggatcacttgaggccatgagtttgagaccagcctgctcaacatgatgaaacttctatctctactaaaaaaaaaagtgggattaggtcaggcacggtggctcacacctgtaatcccagcactttcagaggccgaggcaggaggatcatgaggtcaggagatcgagaccatcctggctaacacggtgaaaccccgtctctactaaaaaaatacaaaaaattagccaggcgtggtggcgggtgcctgtagtcccagctactcaggaggctgaggcaggagaatggcgtgaacccgggaggcggagcttgcagtgagccaagatcgtgccactgtactccagcctgggcgacagagcaagactctgtctcaaaaaaaaaaaaaaaagtgggattgacattctcttcaaagttctggggttttcctttgcaaagacaggattggcaaggccagtgggtcttttttgtgtgtgtgtgtgtgacggagtctcactctgccacccaggctggagtgcaatggcaggatctcggctcaccgcaacctcctcctcccaggttaaagtgattctcctgcctcagcctcccgagtagctgggactacaggtgcccgccaccacacccaactaatttttgtatttttagtagagacagggtttcactatattggccaggctggtcttgaacccctgacctcacgtgatccacccgccttggcctcccaaagtgctgggattacaggcgtgagccactgtgctcggcctcagtgggtctttcctttgagtgacagttcaatcctgtctcttctgtagtgtctgtcacctgcaaatccggggacttcagctgtgggggccgtgtcaaccgctgcattcctcagttctggaggtgcgatggccaagtggactgcgacaacggctcagacgagcaaggctgtcgtaagtgtggccctgcctttgctattgagcctatctgagtcctggggagtggtctgactttgtctctacggggtcctgctcgagctgcaaggcagctgccccgaactgggctccatctcttgggggctcataccaagcctcttccgcccttcaaatccccccttgaccaggaggcattacaaagtggggatggtgctacctcttcgggtttgtcacgcacagtcagggaggctgtccctgccgagggctagccacctggcacacacactggcaagccgctgtgattcccgctggtcgtgatccccgtgatcctgtgatccccgccccgtgaggctgaacacatagtgacgcttgctagccaagcctcaatgacccacgtaacatgaagggggaaaagccagaaagttctgccaaggagcaaggccaagaatcccgaagggaaatggactttgaagctgggcgtcttcttggctgtcttaatacaagtggcacatccaaatccaaaaccccgaaattcaaagtcttgagcacccgaaattctgaaacgtcttgagcactgacctttagaaggaaatgcttattggagcattttggatttcggatttttaccactgagtgtggagtcctaattaggaaaaaaaccaggctgaccgaaccaaaggaaagcaataaaagaaggcagatagggtcaggcacggtggctcacccctgtaatcccagccttttgagaggctgaggcgggtggatcacttgaggtcaggagttcgagagcagcctggccaacacggtgaaaccccatctctactgaaaatacaaaaactagccaggtatggtggcgtctgcctgtaatcccagctactcgggaggctgagacaggagaatcacttgaacctgggaggcagaggttgcagtgagccaatatcacgccattgcactccagcctgggggacaagagcgaaattctgtctcaaaaaaaaagaagaagaaggccgacaaactatgtaactctgcctttctccatggtccagaacacacagccctcctgcgtaaataactccttatcttcctgctcccagctatcatcagacacctcggctgatagaaaattgcaagttagctcactgcaacctcggcattataagtactgcacaaagccctcttcagcgcacagcacaagcaccattctataaaatctccagcaagcggccaggtgcagtggctcatacctgtaatcccagcattttgggagactgaggcgggcggatcacctgaggtcaggagtttgagaccagcctggccaacatggtgaaaccccgtctctattaaaaatacaaaaaaattagccaggcgtggtggcaggtgcctgtaatcccagctacttggaaggctgaggcaggagaatcgcttgaacccgggaggtggaagttgcagtgagccgagatcttgccatcgcactccagcctgggggacaagagtgagacttcgtctcaaaaaaaaaaaaaaaaattcccagcaagcctttgtcttctggcagtcagctcctctcttgctgacctgctcattgctttcttgcaaggtattttcctacctactttctggaataaatctgtctttctgtacttacaactaccttttttaaaatttctttcttttttgagatggagtctcactctgtttgcccaggctggagttcagtggtgcaatctcagctcactgcaacctctacctactgggttcaagcgattctcctgcctcagcttcccgagtagctgggattacaggcgtgcaccagcacgcaggctaatttttgtatttttagtagagacggggtttcaccatgttggccaaggtggtcttgaactcctgacctcaagtgatcctcccacctcagcctcccaaagcgctaggattacggccatgagccactgaggccggctgcacctacaactgtcttgataaattcttacccccacaccactggtccagatagtcagtgctcacccacaacattaaggatattccaaatttgaaacattccaaaatcagaaaaatattccaactctgaaaatattccaaaatccaaaaaaattcaaaatccaaaacacttctggtcccaagcattttagagaagggatactcaacccaaaataaggacagcaattctataaattgtgctaccatcttgcaggtctcagtttaacagctttacacctattagcgcaccagtgctcatagcagtgctgggaaatgtgtacagatgaggaaactgaggcaccgagagggcagtggttcagagtccatggcccctgactgctccccagcccgcctttccaggggcctggcctcactgcggcagcgtccccggctatagaatgggctggtgttgggagacttcacacggtgatggtggtctcggcccatccatccctgcagcccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcggtatgggcggggccagggtgggggcggggcgtcctatcacctgtccctgggctcccccaggtgtgggacatgcagtgatttaggtgccgaagtggatttccaacaacatgccaagaaagtattcccatttcatgtttgtttcttttttttcttttctttctttattttgtttttgagatggagtctcactctgtgatttttttcatctctaaatttcctacatccatatggccaccatgaggccccaggctggccgatggttgctgttagcttattgggaaatcactgtttggaaggtgctggttgttttttgttgtttgttgtttttgtttttgtttttgttttgagacggagtctcgctctgtcgccagggtggagtgcagtggcgcgatcagctcactgcaacctccgcttcctgggttcaagccattctcctgcctcagcctcccaagtagcgcggattacaggcatgtgccaccacctccggctatttttttttctatttagtagagatggggtttcaccatgttagtcaggctggtcatgaactcttgacctcaggtgatccacccgcctcggcctcccaaagtgctgggattacaggcgtgcactgctgcacccagcctttttttgtttttttgagacagggtcttgctgtcacccaggttgaagtaaggtggcacgattatggctcactgcggccttgatctccttggctcaagcgatcctctcacttcagcctctcaagcagttggaaccacaggctgtaccaccaagcctggccaatttttttgtacagacacaggctggtcttgaactcctgggctcaagcaatcctcctgccttggcctcccaaagtgctgggattccaggcatgagccgctgcacccggcaaaaggccctgcttctttttctctggttgtctcttcttgagaaaatcaacacactctgtcctgttttccagctgtggccacctgtcgccctgacgaattccagtgctctgatggaaactgcatccatggcagccggcagtgtgaccgggaatatgactgcaaggacatgagcgatgaagttggctgcgttaatggtgagcgctggccatctggttttccatcccccattctctgtgccttgctgcttgcaaatgatttgtgaagccagagggcgcttccctggtcagctctgcaccagctgtgcgtctgtgggcaagtgacttgacttctcagagcctcacttccttttgttttgagacggagtctcgctctgacacccaggctggagtgctgtggcacaatcacagctcacggcagcctctgcctctgatgtccagtgattctcctgcctcagcctcccgagtagctgagattaaaggcgtataccaccacgcccggctaattttttgtatttttattagagacagggtttctccatgttggccaggctggtcttgaactcctggtctcaggtgatccacccgcctcggcctcccaaagtgctaggattacaggtgtgagccactgcgccaggcctaatttttttgtatttttagtagagatgcggttttgccatattgcccaggctggtctcgaactcctgggctcaagcgatctgcctgccttggcctcccaaagtgctgggattacaggcacaaaccaccgtgcccgacgcgttttcttaatgaatccatttgcatgcgttcttatgtgaataaactattatatgaatgagtgccaagcaaactgaggctcagacacacctgaccttcctccttcctctctctggctctcacagtgacactctgcgagggacccaacaagttcaagtgtcacagcggcgaatgcatcaccctggacaaagtctgcaacatggctagagactgccgggactggtcagatgaacccatcaaagagtgcggtgagtctcggtgcaggcggcttgcagagtttgtggggagccaggaaagggactgagacatgagtgctgtagggttttgggaactccactctgcccaccctgtgcaaagggctccttttttcattttgagacagtctcgcacggtcgcccaggctggagcgcaatggcgcgatctcggctcactgcaacctctgcctcccaggttcaagtgattctcctgcctcagcctcctgagtagctgggattacaggcgcccaccaccaagcccgggtaattttttgtatgtttagtagagatggggtttcactatgttggccaggctggtgttgaactcctgacctcatgatccgcccacctcggcctcccaaagtgctgggattacaggcgtgacccaccccatgaaaaaaaattaaaaaatgaagcgatgctgggcgcggtggatcacgcctgtaatcccagcactttgggaagctgaggcaggcagatcacgagggcaggagattgagaccatcctggctaatacggtgaaaccccatctctactaaaactacaaaaaattagccgggtgtggtggcaggcacctgtgatcccagctactcaggaggctgaggcaggagaatcgcttgaacccaggaggtggaggttgcagtgagccgggatcacaccattgcactccagcctgggtgacagagtgagactctgtctcaaaaaaaaaaaaaaaaaaaaaagcgaattctgaaatacatgaattcttttccttagatgcctgcttctgtcttgaggtttgttgttgttatttcgaaacagagtcttgctctgtcgctcaggctggagtgcagtggcatgatcttggctcaccacaacctccggctcccaggttcaagcgattcttctgcctcagcctcctgagtagctgggattacagctgaatgccaccttgctgggctaatttttgtatttttagtagagatggggtttcaccatgttggccaggctggcctcgaactcctgacctcgagtgatctgcccgcctcctgaagtgctgggattacaggcgtgagccacctcgtcctggtgagggtttttttttttccccaaccctctgtggtggatactgaaagaccatattaggataactgtacagtatagagaaggcagtggcaagttttctctgtcatataccagagtgggcttgggcatggtggcatactcctgtagtctcagctaatcaggaggctgaggaaggaggatcgcttgggcccaggagttggagactgtagtgagctgtgatcacaccaccacacttcaatctgggcaacagagcaagagaccctatctctaaaaaaaagtaagtatttcggacactgtgggccatacggtctctggtgcagtttctcaacatggctgttgggtgaacacaaccacgcacagaacgcaaaccaatacacgtggctgtgggcccagaaaatgttatttatggacacaaaaattggaatttcatataactgttttgtgtcatgaaaatgatttccctttttatttttatttttcttctcaagtatttaaatatgtaaaagccatttttaggcctggcaggatggttcacagctgtaatcccagcactttgggaggtcgaggcgggaggatcacgaggtcaggagatcgagaccatcctggccaacacagtgaaaccccgtctctactaaaaatacaaaaaattaaccaggcttggtggcgcgcgtctgtagtcccagctgctcaggaggctgaggcaggagaatcgcttgaatgcaggaggcggaggttgtagtgagccgaggttgcaccactgcactccagcctgagcgacagagtgagagtccgcctcaaacaaaaaaatgtttgcccatgctggtcttgaactcctgggctcaagctatctgcctgccttggtctcccaaagttctgggattacaggcatgagctacagcgcccggacttttgttgttttatatctatatatctatatataacttgttttatgtatatatataacttgttttatatatatacataaactgcagtaaaaaacatgtaacataaaatttaccttctcaaaccttattaagtgcacagttctgtgccattagcaaattcacactgttgtacaacatcacaaccaccatctccagaacttttttttttttttttattctttttgagacagagtctcactcgtcgcacgggctggagtgcagtggtgcgatctcggttcactgcaacctccacctaccaggttcaagcaattctcctgcctcagccccctcagtagctgggattacaggtgcccgtcctaccacgcccagctaatttttgtattttcagtagagactgactgggtttcaccatgttggccaggctggtctcgaactcctgacctcaagtgatcctcccacctcagcctcccaaagtgctgggaatacaggcatgagccactgcgcccggccccagaactcttttatcttcccaaactgaagctctgtccccatgaaacactcactctccatcccctccccaactcctggcacccaccattctactttctgtccctatgaatgtgatggctctagggacctcctctgagtggaatcagacagcattttccttttttgactggcttatttcactgagccaagtgcggtggcacacgcctgtaatcccaaaactttgggagaccgaggcgggcgcatcacctgaggtcaggagttcgagaccagcccggccaacatggtgaaaccccatctctagtaaaaatacaaaaaattagcctgtcatggtcgtgggtgcctgtaatcccagctaagtgggaggctgaggcaggagaatcgcttgtacccaggaggcggaggtcgcagtgagccgagatcgtgccattacactccagcctgggcaacaagagtgaaactccgtctctcctaaaaatacaaaaaaattagctgggcatggtggcacatgcctgtagtcccagctacttgggaggctgaggcaggagaatcacttgaacccgggaggtggaggttgtaatgagccaaggttggcggcgaagggatgggtaggggcccgagagtgaccagtctgcatcccctggccctgcgcagggaccaacgaatgcttggacaacaacggcggctgttcccacgtctgcaatgaccttaagatcggctacgagtgcctgtgccccgacggcttccagctggtggcccagcgaagatgcgaaggtgattcccgggtgggactgagccctgggccccctctgcgcttcctgacatggcaaccaaacccctcatgcctcagtttccccatctgttaagtgtgcttgaaagcagttaggagggtttcatgagattccacctgcatggaaaactatcattggctggccagagtttcttgcctctggggattagtaattaagaaatttcaggccgggtgcgtaatccctgtaatcccaacaccttgggacgccgaggcgggcagatcacctgaggtcgggagttccagaccagcctgaccaacatggagaaaccccgtctctactaaaaatacaaaattagccgggcttggtggtgcatgcctataatcccagctactcaggaggctgaggcaggagaatcacttgaacctgggaggtggaggttgtggtgagccaagatcgtgccattgcactccagcctgggcaacaagagtgaaactccatccaaaaaaaaaagaaaagaaaagaaaaaaaagaaaagaaatttcagctgacacagcttcacactcttggttgggttcccgtggtgaatgatgaggtcaggtgatgactggggatgacacctggctgtttccttgattacatctcccgagaggctgggctgtctcctggctgccttcgaaggtgtgggttttggcctgggccccatcgctccgtctctagccattggggaagagcctccccaccaagcctctttctctctcttccagatatcgatgagtgtcaggatcccgacacctgcagccagctctgcgtgaacctggagggtggctacaagtgccagtgtgaggaaggcttccagctggacccccacacgaaggcctgcaaggctgtgggtgagcacgggaaggcggcgggtgggggcggcctcaccccttgcaggcagcagtggtgggggagtttcatcctctgaactttgcacagactcatatcccctgaccgggaggctgtttgctcctgagggctctggcaggggagtctgccgccctgttaggacttgggcttgccagggggatgcctgcatatgtcctagtttttgggaatatccagttaacggaaccctcagccctactggtggaacaggaaccggctttcctttcagggacaacctggggagtgacttcaaggggttaaagaaaaaaaattagctgggcatggtgccacacacctgtggtcccagctactcagaaggctgaggcgggaggattgcttgagggcaggaggattggttgatcctcccacctcagcctccggagtagctgggacctcaggtgcatgccactatgcctggctaattttcttttttctttttttttttttttcgagacggagtctcgctctgttgcccaggctggagtgcagtggcaggatctcggctcactgcaagctccgcctcccgggttcacgccattctcctgcctcagcctccccagtagctgggactacaggagcccgccactgcaccaggccaatttttttgtatttttagtagagacggggtttcactgtgttagccaggatggtctcgatctcctgacttcgtgatccgcccacctcggccttccaaagtgctcggattacaggcgtgagccactgcgcccagccgctaattttcatatttttagtaaaaacagggtttcaccatgttggccaggctagtcttgaactcctgaacccaagtgatcctcctgccttggcctcccaaagtgctgggattacagacaccacacctggctattattattttttagagacagggtgctgctctatcttccagcctgtagtgcagtgcagcctccatcatagctcgctgcagccttgacctcctgggttcacgtgatcgtcccgcctaagcctctggaggagctgggagtactggcatgtgccaccatgcctggttaatttttttttttttttttttgagacagagtctcattctgtcacccaggctggagtgcggtggtgcgatcttggcttactgaaacctccacctcccaggttccagcaattctcctgcctcacccttctgagtagctgggattacaggttccggctaccaaacctggctagtttttgtatgtttagtagagacagggtttcaccatgttggtgaggctggtctcgattctcccgcctcagcctcccaaagtgctgggattacaggcttgagccaccgtgcctggcttttttttttttttttttttttgtggcaataaggtctcattgtcttgcccaggctagccttatgctcctagcctcaagtgatcctcctccctcagcctcccaaagtgctgggattacaggtgggcgccactgtgcctgttcccgttgggaggtcttttccaccctctttttctgggtgcctcctctggctcagccgcaccctgcaggatgacacaaggggatggggaggcactcttggttccatcgacgggtcccctctgaccccctgacctcgctccccggacccccaggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcaggtgagcgtcgcccctgcctgcagccttggcccgcaggtgagatgagggctcctggcgctgatgcccttctctcctcctgcctcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatgggtgcgtatccacgacgctgagggctgcagagggaatggagggagcaggaaggagcttcaggaactggttagtgggctgggcatggtggctcaaagcacctgtaatcccagcactttgggaggccaaggtgggtggatcatcaagaccagcctgaccaacatggtgaaacctcgtctctactaaaaatacaaaaattagccgggtgtggtggtgggcacctgtaatcccagctgctcgggaggctgaggcaggagaatcacttgaacctgggagatggaggttgcagtgagccaagacagccccactgcactccagcctgggtgacagagtgagactccgtctcaaaaaaaaaaaaaaaaactaaacaaaaaactggttagtggctagacaacaggatggtatcttccaagcccatggctgactcagcagctcctgggtcaagacactgtgacctgtgtcccctggcaggaagcatcgcccctgccacctgcccggtgtactctgtacctgtcaggtgacatctgctacctaagcacgtgagaggtggcatttcacagtttcagtgtggtgctgacaacccgggacgcacactgtccttgcagctacaatcaggaggtgaatgttgggtttccagcagagaacactggagaaggcacacttggtgtctggaagggaaaagcagggaagagagcatcatcagatgcctgcgggtgaaggtgggcccgctatggccagcgtccctttttatttttatttatttatttatttgagatggaatctcgctctgtcgcccagactgtagtgcagtggtgcgatcacggctcactgcaagctccgcctcacaggttcacgccattctcctgcctcagcctcccgagtagctgggactacaggcacccgccaccacgcccggttaattttttgcatttttattagagacggggtttcaccgcgttagccaggatggtctaaatctcctgaccctgtgatccacccgcctcggcctccctaagtgcttggattacaagcgtgagccaccacgcccggccccctttttattttttattttttgagacggagtctcgctctgtcgcccaggctagattgcagtggcgtgatctcggctcactgcagcctccgcctcccaggttcaagtgattctcctgcctcaacctcccaactaattaggattacaagcatgtaccaccatgcctgactaattttttgtatttttagtagagactgggtttcaccatgttggctaggctggtctcgaacccttagcctcaagtaatctgcctgcctcagcctcccaaacagcggggattacaggcatgagccactgtgcccaacccaaccctggatctcttttaaacaagacaatgctcgctgttgccacagaacaatgggtggggtacatgtggcccagtgtgtttggccacataactgccaggccagagggaaagagactctcagactgtctccactcagatacaaatgtgtgtgttgtgtgcgtgtgttctggtctcatatttgtttgttttgagacagggtgtcgctctgtcactgagtctggagtgcagtggcgcaatcagagttcactgcagcctcaaactcttgggctcagttgattctcccacttcagcctcccaagtagctggaactacaggtgaacaccactgtgcccagctaatttattttatttttagtagagatgaggtctcactatgttgcccaggctggtcttgacctcctagcctcaagcaatcctcctgccttggtctcccaaagtgctgggattacacgtgcgagccattgcgcatggcttgtgttcttgtgtttcttcctttttctttcgagatggcgtctcagtctgccacccaggctggagtgcagtggtgtgatcatagctcactgtagcctcaacttcctgggctcaagcaatcctcttgatttcagcctcccgggcctggccagcatggtgaaaccccgtctctactaaaaatacaaaaatgtagccaggcgtggtggtgggcgcctgtaatcccagctacaccagaggctgaggcaggagaatcgcttgagcctggaaggtggaggttgcagcaagccaagatcgtgccactgcactccagcctgggcaacagagacagactctgtctcaaaaaaaaaaaaaaaaaacccaaacaagccacatttggagtttggggttcccagcaggactatttcccaagcctgagcctggctgtttcttccagaattcgttgcacgcattggctgggatcctcccccgccctccagcctcacagctattctctgtcctcccaccagcttcatgtactggactgactggggaactcccgccaagatcaagaaagggggcctgaatggtgtggacatctactcgctggtgactgaaaacattcagtggcccaatggcatcaccctaggtatgttcgcaggacagccgtcccagccagggccgggcacaggctggaggacagacgggggttgccaggtggctctgggacaagcccaagctgctccctgaaggtttccctctttcttttctttgttttttctttttttgagatgaggtcttggtctgtcacccaggctggagtgcactggcgcaatcgtagctcactgcagcctccacctcccaggctcaagtgatcctcctgcctcaccctcctgagtagctgagattacagacacgtgccaccacggcagactaattttattttatttttgggaagagacaaagtcttgttatgttggcctggctggtctcaaactcagggtgcaagcgatcctcccgcctcagccttccaaactgctgggattacaggcgtgggccaccgtacccagcctccttgaagtttttctgacctgcaactcccctacctgcccattggagagggcgtcacaggggaggggttcaggctcacatgtggttggagctgcctctccaggtgcttttctgctaggtccctggcagggggtcttcctgcccggagcagcgtggccaggccctcaggaccctctgggactggcatcagcacgtgacctctccttatccacttgtgtgtctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggtgtggcttacgtacgagatgcaagcacttaggtggcggatagacacagactatagatcactcaagccaagatgaacgcagaaaactggttgtgactaggaggaggtcttagacctgagttatttctattttcttctttctttttttttttttttttgagacagagttttgctctcgtttcccaggctggagggcaatggcatgatctcggctcaccgcaacctccacctcccaggttcaagtgattctcctgtctcaggctccccagtagctgggattacaggcatgcaccaccaccatgcccggctaattttgtatttttagtagagacggagtttctccatgttggtcaggctggtctcgaactcccgacctcaggtgatctgcctgcctcggcctcccaaagtgctgggattacagacttgagccaccgcgcccagctatttctgttttctttctttcttcttcttctttttttttttctaagagacaggatctcactctgtccccaggcaggagtgcagtgctgtgatcatagctcactgcagccttaacctcctgggctcaagtgatcttcccacctcagcctcccaagtagctggaactacaggtgcacaccaccatgcccagctcatttttgtattttttttttttttgagacagtctcgttctgtcaccccggctggagtgcagtggtacaatcttggctcactgcaacctctgcctcccaggttcaagcgattctcctgcctcagcctcctgagtagttgagattacaggcatgtgtgccatcatacctggctgatttttgtatttttttttagagatggggtctcagtatgttgaccaggcttgtcttaaactcccggcctcaagtgatcctcccacttcagtctcccaaagtgctgggattacaggcatgagccactgcggccggtttgttttcttttttttttcgttttttggagacggaatttcacctttgttgcccaggatggagtgcaatggcacgatatcgcctcaccacaacctctgcctcctgggttcaaaccattttcctgcctcagccttcttagtagctgggattacaagcatgtgccaccacgcccggctgattttgtatttttagtagagatggggtttctccatgttggccaggctggtctcgaactcctgacctcaggtcattcgcccacctctgcctcccaaagtgctgggattacaggcgtgagccaccgtgcccggtggtttgtattctttttactgagagtcgtgaaaggcagtgatcctctgtcacatgtgatcttggctctcaggggacatttggcaatttctagagattttttggttgtcacaagtcaatggggaagactgttggcatttagtgggtagaggctggtgacgctgctgaacacccagaacagggaagtagcaggccctagatagagccatcgtggggaaaccctgctctaaggaaatggcgctattttataaccccacgttcctggcatgattaccaacagccaaaagtggagtccccccaagtgtgttcgtccatttgcattgcagtaaaggaatagctgaggccgggtaatttataaagaaaagagatttaaactgggtatggcagtttatgcctataatcccagaactttgggaggctgaggcaggaggatcgcttgagtccaggagtgtgagaccgagaccagcctggccaacatgacgaaactctgtctctacaaaaaatacaaaaagtaggccaggcacggtggttcacgcctgtaatcccagcactttgggaggccgaggcgggcggatcacgaggtcaggagatcgagaccatcctggctaacacggtgaaaccccgtctctactaaaaatacaaaaacaaaattagccgggtgtggtggcaggcgcctgtagtcccagctactcgggaggctgaggcgggagaatggcgtgaacccgggaggcggagcttgcagtgagccaagatcgcgccactgcactccagcctgggtgaccgagttgagactccgtctcaaaaaaaaaaaaaaaaaaaaaaatacaaaaagtagccaggtgtggtggcaggcacctgtaatcctgggttctcgagaccgaggcatgagaattgcctgaccccaggaggtggaggctgcagtgagccaagatcatgccactgcactccagcctgggcgacagagtgggactctgtctcaaaaaacaacaaaaaaaaagttctggaaatggatggtggtgatggtgatacttccacaacagcgtgaatctgcttaaggccaccgaactgtgcactcacaaatagtcgagatggtacattttatgttatgtgtatttcaccacaattaaaaactagttgtgggccaggtgtggtggttcatgcctgtaatcccagcactttgggaggtcagagggaggtggatcatgaggtcagcagttcgagaccagccaggccaacatggtgaaaccccatctctactaaaaatacaaaaattagccaggcgtggtggcacatgcctgtagtcccagctacttgagaggctgaagcaggagaatcgcttgaacctgggaggctaagattgcagtgagccgagatcgtgccactgcactccagcctggacgacagagtgagacttcgtctcaaaaaaaaaaccaaaaaaaaaattagctgtgggtcaggcactgtggctcacgcctgtaatcccagcactttgggagaccgaggtaggtggatggcctgaggtcaggagttcgaatccagcctggccaacatggtgaaagcccgtctctactaaaaatacaaaaaattagtcaggtatgttggcacacctgtaatcccagctactcgggaggctgaagcaagagaatcgtttgaacccaggaggtggacgttgcagtgagccgagattgggccactgtactccagcctgggcaacaaaagtgaaactctgtctgaaacaaacaaacaaacaaacaaacagacaaacaaaaaaactagttgtggagagagggtggcctgtgtctcatcccagtgtttaacgggatttgtcatcttccttgctgcctgtttaggacaaagtattttggacagatatcatcaacgaagccattttcagtgccaaccgcctcacaggttccgatgtcaacttgttggctgaaaacctactgtccccagaggatatggttctcttccacaacctcacccagccaagaggtaagggtgggtcagccccacccccccaaccttgaaacctccttgtggaaactctggaatgttctggaaatttctggaatcttctggtatagctgatgatctcgttcctgccctgactccgcttcttctgccccaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacaggtgtggcacacgccttgtttctgcgtcctgtgtcctccaactgccccctcctgagcctctctctgctcatctgtcaaatgggtacctcaaggtcgttgtaaggactcatgagtcgggataaccatacttttcttggatggacacatcagcaccgggcttgacatttacccagttcccctttgatgcctggtttcctctttcccggccccctgaagaggtgatctgatttctgacaggagccctgagggaggaaatggtcccctttgttgacttttctttttctttatttttttcttttgagatttgctgtcacccagcctggaatgcagtggtgccatcttggctcactgctacctctcccactgggttcaagcaattctcctgcctcagcctcccaagtagctgggattacaagcatgcgccaccatgcctggctaagttttgtatttttagtacagacagggtttctccatggtggccaggctggtcttgaactcctgacctcaggtgatcctcccacctctgcctcccgaagtgctacgattacaggcatgagccaccgcgcccatccccctttgttgacttttctcatcctctgagaaagtctcagttgaggccagcacctccctcaagtgaattgaatctcccttttgaacaacaacaaataacaatatgacccagacgtggtggctcacacctgtggtcccagctactcgggaggctgaggtgtgaggattgcttgagcccaggaggtcaaggctacagagagctataatcacaccacttcactccagcctgggggacaaagtgaaaccctgtctgaaaaaaacaaaaaaagaaaaaggaaaaagaaacaatacgatcacaaagtagatattcatagtgtttattttcagtactcttttttttttttttttttttttttgagacggagtcttgctctgttgcccaggctggagtgcagtggcacgatcttggctcactgcagcctctgcctcccaggttcaagcgcttggctcactgcaacctccgcctcctgggttcaagcgcttcttctgcctcagcctccccagtagctgggactataggcacgtcccactacgcccagctaattttttgtattttttagtagagatggggtttcactatgttagccaggatggtctcgatctcctgacctcgtgatctgcctgccttgggctcccaaagtgttgggattatgggcatgagccactgcacctggccttttttttttttttttttgagatggagtttcgctcttgttgcccaggctggagtgcaatggtgtgatctcggctcactgcaacctctgcctcctgggttcaagcaattctcctgcctcagcctcccgagtagctgggattacaggcacctgccaccacgcctggctaatttttgtacttttagtagagacggggtttctccatgttggtcaggctggtctcaaactcctgacctcaggtgatccacccacctcggcctcccaaagttctgggattacagacatgagccaccgcgcctggccgtgtctggccttttttagttatttcttttttttttttttttttttttgagacagagtcttactccgtcgcccaggctggagtgcagcggtgcgatgtctgcgcactgcaagctccgccccctgggttcatgccattctcctgcctcagccttctgagtagctgggactgcaggcgcctgccactacgcccggctacttttttgtatatttagtagagatggagtttcactgtgttagccaggatggtctcgatctcctgactttgtgatccgcccgcctcggcctcccaaagtgctgggattacaggcgtgagccaccatgccaggcttttttttttttttttttttttgagacggagtcttgctctgtcgcccaggctggagtgcagtgccatgatctcagctcactgcaagctccacttcccaggctcacgccattctccagcctcagcctcccaagtagctgagactacaggggcccgccaccacactcggctaatttttttgtatttttagtagagacggggtttcaccatgttagccaggctggtcttgaactcctaacctcaggcgattcacctgcctcggcctcccaaagtgctgggattaaaggtatgagccacctcgcctggtgtgagccacctcgcccagcctgagccacctcacccagcctaagccactgtgcctggcctgattttggactttttaaaaattttattaataattatttttgggtttcttttttttgagacagggtcttactctgtcatccaggccatcctgtctgtctgtcatcccagtgatgggatcataccttgctgcagcctctacctcctgggctcaagcgatcctcccccctcagcctcctgagtagctgggagtacaggtgtgcaccaccacacctggctaatttttttttttttttttgtatatagagatggtattttgccatgttgaccaggctagtcttaaactcctggactcactcaagagatcctcctgccttggcctcccaaggtcatttgagactttcgtcattaggcgcacacctatgagaagggcctgcaggcacgtggcactcagaagacgtttatttattctttcagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaaggtaaagactgggccctccctaggcccctcttcacccagagacgggtcccttcagtggccacgaacattttggtcacgagatggagtccaggtgtcgtcctcactcccttgctgaccttctctcacttgggccgtgtgtctctgggccctcagtttccctatctgtaaagtgggtctaataacagttcttgccctctttgcaaggattaaatgggccaaatcatatgaggggccaggtccttcaggctcctggttcccaaagtcagccacgcaccgtgtgggtcccaaaattttatcaaggcacattcgttgcctcagcttcaggcatctgcccaaaaaggccaggactaaggcaaggagagggagggattcctcagtactcagcttttcacagaggctccaaaaggctaaggaatccagtaacgttttaacacaattttacaatttttttttttgagacggagttttgctcttgttgcccaggctggagtgcagtggcacgatctcggctcactgcaacctctggctcccgggttcaagcgattctcctgcctcagtctcccgagtagctgggattacaggcatgcgccaccacgctcggctaattttgtatttttagtacagaaggggcttctctgttggtcaggctggtcgtgaactctcaacctcaggtgagccacccgcctgagcctcccaaagtgctgggattacaggtgtgagccaccacgcctggccttttttttgagacagagtctcgctctcgcccatgctgtactgcagtgacgcagtctgggctcactgtaacctccgcttcccaggttcaagtgattcttctgccgcagcctcccatgtagagtagctgggattacaggcacccgccaccatgcctggctaattcttgcatttttagtagagatggggtttcacagtgttggccaggctggtctcaaacttctgacctcaagtcatctgcctgccttggccctgccaaagtgctgggattatagatgtgagccaccgcgcctggcctacagtttattctttggtggctcacacctgtaatctcagcactttgggaggccaaggtgggagaatggcttgagcccaggagttcaagtccagcctgggcaacatagcaagaccctatctctactacaaaataaataataaataaactaattttttttcttttaaaacccaactattcaacatggcaatgcaatatattaaaaaaattttttttttctttgaaacggagtctctcactgtcacccgggctggagtgcagtgtcgccatcttggctcactgcaacctccgcctcccaggtccaagtgattctcctgcttcagcctcccgagtagctgggattacaggcacccaccaccatacccagctaatatttttgtatttttagtagagatggggtttcactatgttgggcaggctggtctggaactcctgacctcgtgatctgcccgaggatcggcggcctcccaaagtgctggggattgcaggcatgagccaccgtgcccagccaaaacttttttatttttatttttttgggacacggtctcactgtgtaccccagactggagtgatagagtgctgtcatggctcactgcagcctcaacctccctgggctcaggtgatcttcctgcttcagtctcccaggtagctgggactacaggcatgagccaccacacccagctaatttttgaatttttttgtagagacagggtttcaccttgtggcccagacttgtctctaactccagggctcaagcgatctgcccaccttggcctcccaaagtgctgagattaatgcaatttaaaaaattttttggccaggcctggtggctcatgcctgtattcacaacaccttgggaggcaaaggtgggcagatcacttgaggtcaggagttcgagactagcctggccaacatggtgaaaccccctgtctactaaaaaaatacaaaaattacctgggcacagtggtgggtgcctgtaatcccagctacttgggatgctgagggtggagaattgcttgaacctgggaggcagaagttgcagtaagccaagatcatgccactggactccagcctcagtgacagagcaaaactctgtctccaaaaaaattgttttttttttttttttttcaaatcatcacactacagccaaggcctggccacttacttttgtaaataaagttttattggagccagtggaccagtgaggccgaatcttgcaggtgtaagatcacagtctatccttgaaaattttgatattttgttcattgggtggtttttcattaatttaaattttaaaaaataacatattaaaggctggtgtggaggtgcacgcctgcagtcctagctactcccagaggctgaggcgggagacttgcttgagcccaagagttgaagtccagcctgggcaacatagcgagacccccatctctaaaaataaaaataatgcattagaatattattggattcctgggcagggcacagtggctcacacctgtaatcccagcactttgggaggctgaggtgggtggatcacctgaggtcaggagtttgagaccagcctggccaacatggtgaaaccccgtctctactaaaaatacaaaaattagccaggcgtggtggcaggtgcctgtaatcccagctactcgggaggctgaagcacgagaatcgcttgaatccaggaggcggaggttgcagtgagctgagattgcgccattgcactccagcctggaggacaagagtgaaactccattcccctctgcaaagaaaaggaatattatcagattcctaagctttttggctccccctttagtttgggggctggggtggtgagtgtctgacctggcctcactgtcctccctggatgtgatgagacccaggtgtgggtcaggatgtcattcgtttgtccaccagagggcgcccaaactgctttgagctgctgggaaatggtgctcctagacttttagcaaacaaacaaaaaaaaatggcacatcggcaaatttcagaccattctttttttttttttttttggttccagagtagctgaaatctttgttcagttacaagcaggataaaatggaaactgcctgggagaggctgagaaaccttcttgcttgggggaggtggggcactgctagaattaatcgcttcacagaccagcccatccaggactcctcaaatttggcaaaaaagccattcattcattcattcatttatgtagagacgagggggatctggctatattgcctagattggtctcaaattcctggcctcaagtgatcctcctgccttggtctactaatgtgctgcgattacaggcatgagccaccgtgcctagctctagtggacttgaaatgttgccttgcccagggcccttatgttgaatggcccaggtccacttgtatggttctgtaccaaggttaaccccatcccataatgcctgggacagttgatgcaggacaatcagcttctgtgccattcaacctcaggactgagcatgctgggcattgtggggtccgaaggtggctcccctgtccccttcaaaataccctctttttcttttcttcttttttttttttttttttttttgagacgaagtcttgctctgttgccccagctagagtgcagtggtgcgatctcagctccccgcaacctctgcttcccgggttcaggcgattctcctgcctcagcctcctgagtagctgggattacaggtgcccaccgccacagctggctaatttttgtatttttagtagagacagggtttcaccgtgttggccaggctggtcttgaactcctgacctcaggcaacctgcccacctcagcctcccaaagtgctgggattacaggtttgagccactgggcctggccttttttttttttttttgagagggagtctcactctgttgcccaggctggagtgcaatggcgcgatcttgactcactgcaactccatttcccgggttcaagtgattctcctccctcagcctcccaagtagctgggattacaggtgcatgccaccacggccagctaattttgtatttttagtagagacagggtttcactatgttgatcatgctggtctcaaactcctgaccttaggtgatctgcccgccttagcctcccaaagtgttgggattacaggtgtgagccaccgcgcccagaccaaaatatgctcattttaataaaatgcacaagtaggttgacaagaatttcacctgcaaccttgtcaaccacctagaataaaagcctctgcagccctcccctaaagactcatcaatgtgaggctcaagaaccttcttaggctgggctcggtggctcatttctgtaatccctgcactttggaaggctgaggcaggaggatctcttgaggccaggagttcaagacaagcctgggcaacatagccagacctctgtttctatcccccacaaaaagaaccttcttaaaccggaattgagtcctacaacctcgataactcacaaataagcccgtgtggcctctcacagacttgggaagttctccaagtgtccagggagatgtgccaggcgctttcctgccgtgaccaccgtcctctgcctgctccatttcttggtggccttcctttagacctgggcctcactcttgcttctctcctgcagctctgggcgacgttgctggcagaggaaatgagaagaagcccagtagcgtgagggctctgtccattgtcctccccatcggtaagcgcgggccggtcccccagcgtcccccaggtcacagcctcccgctatgtgacctcgtgcctggctggttgggcctgttcactttttctcctggacagggaacagccccactggtgtcctttatcacccccacggcctctcctggcttggggctgacagtgacaagatcagacagctaaggggtcagatggaggatgtggagctgggtcccgtgctgtggaatagcctcaccgagatttgagtgccttctggggaactggttcccttgcagggggctgtgtggagaggcgcgctctccctgcctcacccatgctcatcctaactcggttaccatcacatctcttttttctttttttcttaaattttaagaaaaaagaaatttaatttttttgagagacagagtcttgctctgtcacccaggctggagtgcagtggcaccatcatgcctcgctgcagcctcaatgtctgggctcaagcgatcctcccacctcagcctcctgagtagctggtgcaagccactataccccacttcctatttcttaaaaagtcacagccctgtgtgtggctaatcctggacagaaatctagaagaagtcagctacttctggggcgtggctcacccagtgggcttcaggttagatatttcttatacttatgaggctgggtgtggtggcttatgcctgtaatcccagcactttgggaggctgaagtgggtggattgcttgggctcaggagttcgagaccaacctgggcaacatggcgaaaccctgtttctagaaaaggtacaaaaattagctgggcaggtggcacgtgcctgtggtaccagctacttgagggcctgaggcaggaggatcgcttgaacctgggaggtcgaggttgcagtgaactgagatcatgtcactgcactccagcctggtgacagagcaagaccccgtctcaaaaaaaaaaaaagaaagaaaaaaattcttatgcatagatttgcctcttttctgtttgtttgttttgagatggagtctcgctctgtcgcccaggctggagtacagtggctcaacctcggctcactgcaacctctgcctcccgggttcaagcaattctcctgcctcagcctcctgagtagctgggactacaggcgcccgccaccatgcccagctaatttttgtatttttagtagagactgactgggtttcatcatgttggccaggctggtctcgaactcttgacctcatgatccgcccgcctcagcctcccaaaatgctgggattacaggcgtgagccaccaggcccaggccgcaaggcgatctctaaacaaacataaaagaccaggagtcaaggttatggtacgatgcccgtgttttcactccagccacggagctgggtctctggtctcgggggcagctgtgtgacagagcgtgcctctccctacagtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcggtgagtgaccctctctagaaagccagagcccatggcggccccctcccagctggaggcatatgatcctcaagggaccaggccgaggcttccccagccctccagatcgaggacagcattaggtgaatgcttctgtgcgctcattcagaatgtcagcggacaatggccttggtggtgtagaggaatgttggataagcaaatagagagctccatcagatggtgacagggcaaagaaagtcaaaaggagttcagaggccgggcgcggtggctcatgcctgtaatcccaggactttgggaggccgaggctggcggatcacctgaagtcaggagtttgagaccagcttggccatcatgacaaaaccccgtctctattaaaaatacaaaaaattagccaggcgtgggagtgggcgcctgtaatcccagctactcgggaggccgaggtagaaaaatcgcttgaacctaggaggcagaggttgcagtgagccgagatcgcgccactgcattccagcccgggaggcaagagcaaaactccatctcaaaaaaaaaaaaaaaaggagttcagaggcccggcatggtggttcacacatgtgatcccagaacttggggaggttgaggcaggagaatcacctgagctcagagttcaagaccagcctgggcagcacagcaagaccccatctctgcaaaaaataaaaatttagcccagtgtggtgatgagcgcctagttccagctactagggaggctaaggcaggaggattgcttgaggctaaggtaggagattgagactgcagtgacttgtgattgcgtcactgcgctccagcctgggtgacagagcaagcccttgtctcttaaaaaaaaaaaaaaattcaaagaagggtttccagagggccaggagggaggaagggagaggaggtgttttatttttttgcttttattttttattttgagacagagtctctctctgtcacccaggttggagtgcagtgctgtgatcttggctcactgcaacttctgcctcctgggttcaagcaattcttatgcctcagcctcagcctcctgagtagctgggattacaacactatgcccgggtaatttttgtatttttagtagagacgaggtttcgccatgttgcccagactggtctcgaactcctgacctcaagtgatccacccgccttggcctccccacgtgctgggattgcaggcgtgagccactgcgcccgccttgatctttacacaaggggtttagggtaggtagccttctctgaaccaggagaacagcctgtgcgaaggccctgaggctggaccgtgcctgttgggtttgaggccgttgtagctggagcaaacagagagaggggtaaaaaggcaggaggctaccaggcaggttgtgcagagccttgtgggccactggggaggactttggcttttgccctgagagcggtgggaagtgactgaatccggtactcaccgtctccctctggcggctcctgggggaacatgcttggggatcaggctgggggaggctgccaggcccaggaggtgagaagtaggtggcctccagccgtgtttcctgaatgctggactgatagtttccgctgtttaccatttgttggcagagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgta

SEQ ID NO: 5 - RNA, 3635 nt, Homo sapiens
  source 1..3635: mol_type = mRNA
ttgcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgccatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgccttcccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgggccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 6 - RNA, 1101 nt, Homo sapiens
  source 1..1101: mol_type = mRNA
aggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgcatatttggaggatcactgcgggggccacagaggtgctgttcagatggcacttcagaagactcaggagaccctggggcaggagcagtttgactgacagcccagagggctgccctctgattccacctgaggccctgcttttcctggctgcaggggttccagggccaggccatttccgctggcgcaggactctgctagcagcaacctgcctgaagtcttcctttggcctggctgagagtttctgagacctgcgctggagcggagacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctc

SEQ ID NO: 7 - RNA, 2079 nt, Homo sapiens
  source 1..2079: mol_type = mRNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtga

SEQ ID NO: 8 - RNA, 2333 nt, Homo sapiens
  source 1..2333: mol_type = mRNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtctgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaatttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggctt

SEQ ID NO: 9 - RNA, 2768 nt, Homo sapiens
  source 1..2768: mol_type = mRNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgctcccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagtgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccgaagcattgcctgccagagctttgttttatatatttattcatctgggaggcagaacaggcttcggacagtgcccatgcaatggcttgggttgggattttggtttcttcctttcctcgtgaaggataagagaaacaggccc

SEQ ID NO: 10 - RNA, 2429 nt, Homo sapiens
  source 1..2429: mol_type = mRNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtcctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagtctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccagccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctc

SEQ ID NO: 11 - RNA, 2656 nt, Homo sapiens
  source 1..2656: mol_type = mRNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttc

SEQ ID NO: 12 - RNA, 3144 nt, Homo sapiens
  source 1..3144: mol_type = mRNA
gccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctttactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgt

SEQ ID NO: 13 - RNA, 3522 nt, Homo sapiens
  source 1..3522: mol_type = mRNA
ccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtaaaaaaaaaaaaaaaaaaaaaaa

SEQ ID NO: 14 - RNA, 2600 nt, Homo sapiens
  source 1..2600: mol_type = mRNA
cagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaaca

SEQ ID NO: 15 - RNA, 2583 nt, Homo sapiens
  source 1..2583: mol_type = mRNA
atggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtag

SEQ ID NO: 16 - RNA, 5356 nt, Homo sapiens
  source 1..5356: mol_type = mRNA
gaggggaggcctggcgttcctccgcggttcctgtcacaaaggcgacgacaagtcccgggtccccggagccgcctccgcgacatacacgagtcgccctccgttatcctgggccctcctggcgaagtccccggtttccgctgtgctctgtggcgacacctccgtccccaccttgtcctggggggcgccctcgccccaccagccccgatcaagttcacagaggggcccccggccaccctcaaggcctcggttccttacgaggttgaaacgttgcctcagaatctccccgcccctccttggtctgcagccgagatcttcagccacgtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggggatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggtgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtatggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaaagaaaaaaaaaaaaaaaaaaaca

SEQ ID NO: 17 - RNA, 3635 nt, Homo sapiens
  source 1..3635: mol_type = mRNA
ttgcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgctttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgccatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgccttcccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgggccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 18 - RNA, 1101 nt, Homo sapiens
  source 1..1101: mol_type = mRNA
aggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgctttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgcatatttggaggatcactgcgggggccacagaggtgctgttcagatggcacttcagaagactcaggagaccctggggcaggagcagtttgactgacagcccagagggctgccctctgattccacctgaggccctgcttttcctggctgcaggggttccagggccaggccatttccgctggcgcaggactctgctagcagcaacctgcctgaagtcttcctttggcctggctgagagtttctgagacctgcgctggagcggagacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctc

SEQ ID NO: 19 - RNA, 2079 nt, Homo sapiens
  source 1..2079: mol_type = mRNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgctttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtga

SEQ ID NO: 20 - DNA, 3635 bp, Homo sapiens
  source 1..3635: mol_type = genomic DNA
ttgcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgccatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgccttcccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgggccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 21 - DNA, 1101 bp, Homo sapiens
  source 1..1101: mol_type = genomic DNA
aggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgcatatttggaggatcactgcgggggccacagaggtgctgttcagatggcacttcagaagactcaggagaccctggggcaggagcagtttgactgacagcccagagggctgccctctgattccacctgaggccctgcttttcctggctgcaggggttccagggccaggccatttccgctggcgcaggactctgctagcagcaacctgcctgaagtcttcctttggcctggctgagagtttctgagacctgcgctggagcggagacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctc

SEQ ID NO: 22 - DNA, 2079 bp, Homo sapiens
  source 1..2079: mol_type = genomic DNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtga

SEQ ID NO: 23 - DNA, 2333 bp, Homo sapiens
  source 1..2333: mol_type = genomic DNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtctgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaatttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggctt

SEQ ID NO: 24 - DNA, 2768 bp, Homo sapiens
  source 1..2768: mol_type = genomic DNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgctcccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagtgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccgaagcattgcctgccagagctttgttttatatatttattcatctgggaggcagaacaggcttcggacagtgcccatgcaatggcttgggttgggattttggtttcttcctttcctcgtgaaggataagagaaacaggccc

SEQ ID NO: 25 - DNA, 2429 bp, Homo sapiens
  source 1..2429: mol_type = genomic DNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtcctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagtctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccagccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctc

SEQ ID NO: 26 - DNA, 2656 bp, Homo sapiens
  source 1..2656: mol_type = genomic DNA
gtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttc

SEQ ID NO: 27 - DNA, 3144 bp, Homo sapiens
  source 1..3144: mol_type = genomic DNA
gccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctttactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgt

SEQ ID NO: 28 - DNA, 3522 bp, Homo sapiens
  source 1..3522: mol_type = genomic DNA
ccagggtttccagctaggacacagcaggtcgtgatccgggtcgggacactgcctggcagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtaaaaaaaaaaaaaaaaaaaaaaa

SEQ ID NO: 29 - DNA, 2600 bp, Homo sapiens
  source 1..2600: mol_type = genomic DNA
cagaggctgcgagcatggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaaca

SEQ ID NO: 30 - DNA, 2583 bp, Homo sapiens
  source 1..2583: mol_type = genomic DNA
atggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtag

SEQ ID NO: 31 - DNA, 5356 bp, Homo sapiens
  source 1..5356: mol_type = genomic DNA
gaggggaggcctggcgttcctccgcggttcctgtcacaaaggcgacgacaagtcccgggtccccggagccgcctccgcgacatacacgagtcgccctccgttatcctgggccctcctggcgaagtccccggtttccgctgtgctctgtggcgacacctccgtccccaccttgtcctggggggcgccctcgccccaccagccccgatcaagttcacagaggggcccccggccaccctcaaggcctcggttccttacgaggttgaaacgttgcctcagaatctccccgcccctccttggtctgcagccgagatcttcagccacgtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggggatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggcaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggtgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtatggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaaagaaaaaaaaaaaaaaaaaaaca

SEQ ID NO: 32 - DNA, 3635 bp, Homo sapiens
  source 1..3635: mol_type = genomic DNA
ttgcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgctttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgccatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgccttcccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgtgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgggccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 33 - DNA, 1101 bp, Homo sapiens
  source 1..1101: mol_type = genomic DNA
aggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgctttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgcatatttggaggatcactgcgggggccacagaggtgctgttcagatggcacttcagaagactcaggagaccctggggcaggagcagtttgactgacagcccagagggctgccctctgattccacctgaggccctgcttttcctggctgcaggggttccagggccaggccatttccgctggcgcaggactctgctagcagcaacctgcctgaagtcttcctttggcctggctgagagtttctgagacctgcgctggagcggagacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctc

SEQ ID NO: 34 - DNA, 2079 bp, Homo sapiens
  source 1..2079: mol_type = genomic DNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgctttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtga

SEQ ID NO: 35 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 36 - protein, 800 aa, Homo sapiens
ETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 37 - protein, 682 aa, Homo sapiens
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPREAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 38 - protein, 692 aa, Homo sapiens
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCLTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 39 - protein, 819 aa, Homo sapiens
MGPWGWKLRWTVALLLAAAGTAVGDRCERNEFQCQDGKCISYKWVCDGSAECQDGSDESQETCSPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 40 - protein, 858 aa, Homo sapiens
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSMVSLEDDVA

SEQ ID NO: 41 - protein, 739 aa, Homo sapiens
MGPWGWKLRWTVALLLAAAGTAVGDRCERNEFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 42 - protein, 692 aa, Homo sapiens
MGTVSSRRSWWPLPLLLLLLLLLGPAGARAQEDEDGDYEELVLALLSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 43 - DNA, 20 bp, synthetic construct
  misc_feature 1..20: note = PCSK9 gRNA
  source 1..20: mol_type = other DNA
cccggtccttggcgcgcggt

SEQ ID NO: 44 - DNA, 20 bp, synthetic construct
  misc_feature 1..20: note = PCSK9 gRNA
  source 1..20: mol_type = other DNA
aggacgaggacggcgacacg

SEQ ID NO: 45 - DNA, 20 bp, synthetic construct
  misc_feature 1..20: note = PCSK9 gRNA
  source 1..20: mol_type = other DNA
ccgtgctcgggtgcttcggc

SEQ ID NO: 46 - DNA, 20 bp, synthetic construct
  misc_feature 1..20: note = PCSK9 gRNA
  source 1..20: mol_type = other DNA
ccggtccttggcgcgcggtg

SEQ ID NO: 47 - DNA, 20 bp, synthetic construct
  misc_feature 1..20: note = PCSK9 gRNA
  source 1..20: mol_type = other DNA
cttcggccggccgtcctcct

SEQ ID NO: 48 - DNA, 20 bp, synthetic construct
  misc_feature 1..20: note = PCSK9 gRNA
  source 1..20: mol_type = other DNA
agccaccccaccgcgcgcca

SEQ ID NO: 49 - DNA, 20 bp, synthetic construct
  misc_feature 1..20: note = PCSK9 gRNA
  source 1..20: mol_type = other DNA
ggttccgtgctcgggtgctt

SEQ ID NO: 50 - DNA, 20 bp, synthetic construct
  misc_feature 1..20: note = PCSK9 gRNA
  source 1..20: mol_type = other DNA
tcccggtccttggcgcgcgg

SEQ ID NO: 51 - DNA, 20 bp, synthetic construct
  misc_feature 1..20: note = PCSK9 gRNA
  source 1..20: mol_type = other DNA
gaggacggcgacacgaggag

SEQ ID NO: 52 - DNA, 20 bp, synthetic construct
  misc_feature 1..20: note = PCSK9 gRNA
  source 1..20: mol_type = other DNA
accgcgcgccaaggaccggg

SEQ ID NO: 53 - DNA, 20 bp, synthetic construct
  misc_feature 1..20: note = PCSK9 gRNA
  source 1..20: mol_type = other DNA
ccgcgcgccaaggaccggga

SEQ ID NO: 54 - DNA, 20 bp, synthetic construct
  misc_feature 1..20: note = PCSK9 gRNA
  source 1..20: mol_type = other DNA
cccaccgcgcgccaaggacc

SEQ ID NO: 55 - DNA, 20 bp, synthetic construct
  misc_feature 1..20: note = PCSK9 gRNA
  source 1..20: mol_type = other DNA
gccggccgtcctcctcggcg

SEQ ID NO: 56 - DNA, 20 bp, synthetic construct
  misc_feature 1..20: note = PCSK9 gRNA
  source 1..20: mol_type = other DNA
ggtggctgtggttccgtgct

SEQ ID NO: 57 - DNA, 20 bp, synthetic construct
  misc_feature 1..20: note = PCSK9 gRNA
  source 1..20: mol_type = other DNA
ccctcccggtccttggcgcg

SEQ ID NO: 58 - DNA, 20 bp, synthetic construct
  misc_feature 1..20: note = PCSK9 gRNA
  source 1..20: mol_type = other DNA
aggacggcgacacgaggagc

SEQ ID NO: 59 - DNA, 20 bp, synthetic construct
  misc_feature 1..20: note = PCSK9 gRNA
  source 1..20: mol_type = other DNA
accgggagggccggcaccac

SEQ ID NO: 60 - DNA, 20 bp, synthetic construct
  misc_feature 1..20: note = PCSK9 gRNA
  source 1..20: mol_type = other DNA
gtggctgtggttccgtgctc

SEQ ID NO: 61 - DNA, 20 bp, synthetic construct
  misc_feature 1..20: note = PCSK9 gRNA
  source 1..20: mol_type = other DNA
ccccaccgcgcgccaaggac

SEQ ID NO: 62 - DNA, 20 bp, synthetic construct
  misc_feature 1..20: note = PCSK9 gRNA
  source 1..20: mol_type = other DNA
gtgccggccctcccggtcct

SEQ ID NO: 63 - DNA, 20 bp, synthetic construct
  misc_feature 1..20: note = PCSK9 gRNA
  source 1..20: mol_type = other DNA
aggagcgggcagccgcgccg

SEQ ID NO: 64 - DNA, 20 bp, synthetic construct
  misc_feature 1..20: note = PCSK9 gRNA
  source 1..20: mol_type = other DNA
cgggagggccggcaccacgg

SEQ ID NO: 65 - DNA, 20 bp, synthetic construct
  misc_feature 1..20: note = PCSK9 gRNA
  source 1..20: mol_type = other DNA
agcgggcagccgcgccgagg

SEQ ID NO: 66 - DNA, 20 bp, synthetic construct
  misc_feature 1..20: note = PCSK9 gRNA
  source 1..20: mol_type = other DNA
ccgggagggccggcaccacg

SEQ ID NO: 67 - DNA, 20 bp, synthetic construct
  misc_feature 1..20: note = PCSK9 gRNA
  source 1..20: mol_type = other DNA
gaccgggagggccggcacca

SEQ ID NO: 68 - DNA, 20 bp, synthetic construct
  misc_feature 1..20: note = PCSK9 gRNA
  source 1..20: mol_type = other DNA
gtccttggcgcgcggtgggg

SEQ ID NO: 69 - DNA, 20 bp, synthetic construct
  misc_feature 1..20: note = LDLR gRNA
  source 1..20: mol_type = other DNA
gtcataggaagagacgccgt

SEQ ID NO: 70 - DNA, 20 bp, synthetic construct
  misc_feature 1..20: note = LDLR gRNA
  source 1..20: mol_type = other DNA
cggataccaagggcgtgaag

SEQ ID NO: 71 - DNA, 20 bp, synthetic construct
  misc_feature 1..20: note = LDLR gRNA
  source 1..20: mol_type = other DNA
tgtcataggaagagacgccg

SEQ ID NO: 72 - DNA, 20 bp, synthetic construct
  misc_feature 1..20: note = LDLR gRNA
  source 1..20: mol_type = other DNA
cgtggtcgctctggacacgg

SEQ ID NO: 73 - DNA, 20 bp, synthetic construct
  misc_feature 1..20: note = LDLR gRNA
  source 1..20: mol_type = other DNA
tgtctctgttgcggatacca

SEQ ID NO: 74 - DNA, 20 bp, synthetic construct
  misc_feature 1..20: note = LDLR gRNA
  source 1..20: mol_type = other DNA
gtccacagccagcccgtcgg

SEQ ID NO: 75 - DNA, 20 bp, synthetic construct
  misc_feature 1..20: note = LDLR gRNA
  source 1..20: mol_type = other DNA
agaccagtagattctattgc

SEQ ID NO: 76 - DNA, 20 bp, synthetic construct
  misc_feature 1..20: note = LDLR gRNA
  source 1..20: mol_type = other DNA
cggtccagtagatgttgctg

SEQ ID NO: 77 - DNA, 20 bp, synthetic construct
  misc_feature 1..20: note = LDLR gRNA
  source 1..20: mol_type = other DNA
cgccgtgggctctgtcaagc

SEQ ID NO: 78 - DNA, 20 bp, synthetic construct
  misc_feature 1..20: note = LDLR gRNA
  source 1..20: mol_type = other DNA
gaacgtggtcgctctggaca

SEQ ID NO: 79 - DNA, 20 bp, synthetic construct
  misc_feature 1..20: note = LDLR gRNA
  source 1..20: mol_type = other DNA
ctactggaccgactctgtcc

SEQ ID NO: 80 - DNA, 20 bp, synthetic construct
  misc_feature 1..20: note = LDLR gRNA
  source 1..20: mol_type = other DNA
agagacatccaggcccccga

SEQ ID NO: 81 - DNA, 20 bp, synthetic construct
  misc_feature 1..20: note = LDLR gRNA
  source 1..20: mol_type = other DNA
tctgctgatgacggtgtcat

SEQ ID NO: 82 - DNA, 20 bp, synthetic construct
  misc_feature 1..20: note = LDLR gRNA
  source 1..20: mol_type = other DNA
gagacatccaggcccccgac

SEQ ID NO: 83 - DNA, 20 bp, synthetic construct
  misc_feature 1..20: note = LDLR gRNA
  source 1..20: mol_type = other DNA
gccgtgggctctgtcaagct

SEQ ID NO: 84 - DNA, 20 bp, synthetic construct
  misc_feature 1..20: note = LDLR gRNA
  source 1..20: mol_type = other DNA
gtctctgttgcggataccaa

SEQ ID NO: 85 - DNA, 20 bp, synthetic construct
  misc_feature 1..20: note = LDLR gRNA
  source 1..20: mol_type = other DNA
ggtcgctctggacacggagg

SEQ ID NO: 86 - DNA, 20 bp, synthetic construct
  misc_feature 1..20: note = LDLR gRNA
  source 1..20: mol_type = other DNA
tggccagcaatagaatctac

SEQ ID NO: 87 - DNA, 20 bp, synthetic construct
  misc_feature 1..20: note = LDLR gRNA
  source 1..20: mol_type = other DNA
catccaggcccccgacgggc

SEQ ID NO: 88 - DNA, 20 bp, synthetic construct
  misc_feature 1..20: note = LDLR gRNA
  source 1..20: mol_type = other DNA
gggtgctgcagatcattctc

SEQ ID NO: 89 - DNA, 20 bp, synthetic construct
  misc_feature 1..20: note = LDLR gRNA
  source 1..20: mol_type = other DNA
tactggaccgactctgtcct